(19) United States
(12) Patent Application Publication
(10) Pub. No.: US 2009/0062268 A1
(43) Pub. Date: Mar. 5, 2009

(54) NOVEL INHIBITORS OF POLY(ADP-RIBOSE)POLYMERASE (PARP)

(75) Inventor: Daniel CHU, Santa Clara, CA (US)

Correspondence Address: WILSON SONSINI GOODRICH & ROSATI, 650 PAGE MILL ROAD, PALO ALTO, CA, 94304-1050, US

(73) Assignee: LEAD THERAPEUTICS, INC., San Bruno, CA (US)

(21) Appl. No.: 12/180,403

(22) Filed: Jul. 25, 2008

Related U.S. Application Data
(60) Provisional application No. 60/968,147, filed on Aug. 27, 2007.

(51) Int. Cl. A61K 31/5386; C07D 221/20; C07D 453/00; C07D 455/02; C07D 235/04; A61K 31/438; A61K 31/435; A61K 31/4184; A61P 9/00; A61P 31/04; C07D 401/02; A61K 31/439; A61K 31/46; A61P 35/00; C07D 265/36; C07D 401/14

(52) U.S. Cl. 514/230.5; 546/16; 546/133; 546/138; 548/305.1; 548/309.7; 544/71; 544/362; 514/278; 514/305; 514/306; 514/304; 546/126; 514/394

(57) ABSTRACT

A compound having the structure set forth in Formula (I):

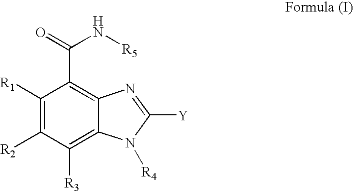

wherein the variables Y, R₁, R₂, R₃, R₄ and R₅ are as defined herein. Compounds described herein are inhibitors of poly(ADP-ribose)polymerase activity. Also described herein are pharmaceutical compositions that include at least one compound described herein and the use of such compounds and pharmaceutical compositions to treat diseases, disorders and conditions that are ameliorated by the inhibition of PARP activity.

### CROSS-REFERENCE

This application claims the benefit of U.S. provisional application Ser. No. 60/968,147, entitled, “Novel Inhibitors of Poly(ADP-Ribose)Polymerase (PARP)” filed Aug. 27, 2007, which is incorporated by reference in its entirety.

### FIELD OF THE INVENTION

Described herein are compounds, methods of making such compounds, pharmaceutical compositions and medicaments containing such compounds, and methods of using such compounds to treat or prevent diseases or conditions associated with the enzyme poly(ADP-ribose)polymerase (PARP).

### BACKGROUND OF THE INVENTION

The family of poly(ADP-ribose)polymerases (PARP) includes approximately 18 proteins, which all display a certain level of homology in their catalytic domain but differ in their cellular functions (Ame et al., BioEssays., 26(8), 882-893 (2004)). PARP-1 and PARP-2 are unique members of the family, in that their catalytic activities are stimulated by the occurrence of DNA strand breaks.

PARP has been implicated in the signaling of DNA damage through its ability to recognize and rapidly bind to DNA single or double strand breaks (D'Amours, et al., Biochem. J., 342, 249-268 (1999)). It participates in a variety of DNA-related functions including gene amplification, cell division, differentiation, apoptosis, DNA base excision repair as well as effects on telomere length and chromosome stability (d'Adda di Fagagna, et at., Nature Gen., 23(1), 76-80 (1999)).

### SUMMARY OF THE INVENTION

Compounds, compositions and methods for modulating the activity of PARP are provided. Among the compounds that are provided herein, are compounds that are inhibitors of PARP.

Compounds provided herein have the structure of Formula (I) and pharmaceutically acceptable salts, solvates, esters, acids and prodrugs thereof. In certain embodiments, isomers and chemically protected forms of compounds having a structure represented by Formula (I) are also provided. Formula (I) is as follows:

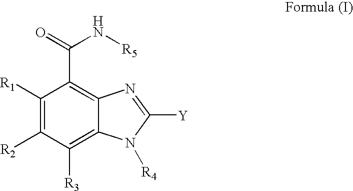

wherein: Y is a non-aromatic 5, 6, 7, 8, 9, 10, 11, or 12-membered bicyclic heterocycle ring having 1 or 2 nitrogen atoms and, optionally, one sulfur or oxygen atom, wherein the bicyclic heterocycle is optionally substituted with 1, 2, or 3 R₆; R₆ is selected independently from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, hydroxyl, hydroxyalkyl, nitro, oxo, heteroaryl, heteroarylalkoxy, heteroaryloxy, heteroarylthio, heteroarylalkylthio, heterocycloalkyl, heterocycloalkoxy, heterocycloalkylthio, heterocyclooxy, heterocyclothio, NR_{A}R_{B}, (NR_{A}R_{B})alkyl, (NR_{A}R_{B})carbonyl, (NR_{A}R_{B)carbonylalkyl, and (NR}_{A}R_{B})sulfonyl and is optionally attached to either one or both of the cyclic rings; R₁, R₂, and R₃, are each independently selected from the group consisting of hydrogen, halogen, alkenyl, alkoxy, alkoxycarbonyl, alkyl, cycloalkyl, alkynyl, cyano, haloalkoxy, haloalkyl, hydroxyl, hydroxyalkyl, nitro, NR_{C}R_{D}, and (NR_{C}R_{D})carbonyl; R_{A}, R_{B}, R_{C}, and R_{D} are independently selected from the group consisting of hydrogen, alkyl, cycloalkyl, and alkylcarbonyl; or R_{A} and R_{B} or R_{C} and R_{D} taken together with the atom to which they are attached form a 3-10 membered heterocycloalkyl ring which optionally contains one to three heteroatoms or hetero functionalities selected from the group consisting of —O—, —NH, —N(C₁-C₆-alkyl)-, —NCO(C₁-C₆-alkyl)-, —N(aryl)—, —N(aryl—C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycloalkyl ring is optionally substituted with one or more substituents; R₄, and R₅ are each independently selected from the group consisting of hydrogen, alkyl, cycloalkyl, alkoxyalkyl, heterocycloalkyl, hydroxyalkyl, and (NR_{A}R_{B})alkyl; and isomers, salts, solvates, chemically protected forms, and prodrugs thereof.

### DETAILED DESCRIPTION OF THE INVENTION

PARP thus has an essential role in facilitating DNA repair, controlling RNA transcription, mediating cell death, and regulating immune response. PARP inhibitors have demonstrated efficacy innumerous models of disease particularly in models of ischemia reperfusion injury, inflammatory disease, degenerative diseases, protection from above adverse effects of cytotoxic compounds, and potentiation of cytotoxic cancer therapy. They have been efficacious in the prevention of ischemia reperfusion injury in models of myocardial infarction, stoke, other neural trauma, organ transplantation, as well as reperfusion of the eye, kidney, gut and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing apoptosis of cancer cells, limiting tumor growth, decreasing metastasis, and prolonging the survival of tumor-bearing animals.

In another embodiment are provided compounds of Formula (I)

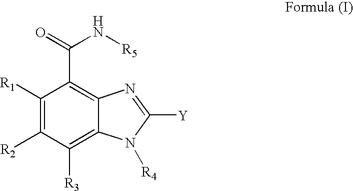

or a therapeutically acceptable salt thereof wherein R₁, R₂, and R₃, are each independently selected from the group consisting of hydrogen, halogen, alkenyl, alkoxy, alkoxycarbonyl, alkyl, cycloalkyl, alkynyl, cyano, halo alkoxy, halo alkyl, hydroxyl, hydroxyalkyl, nitro, NR_{C}R_{D}, and (NR_{C}R_{D})carbonyl; R_{C}, and R_{D} are independently selected from the group consisting of hydrogen, alkyl, cycloalkyl, and alkylcarbonyl; or R_{C} and R_{D} taken together with the atom to which they are attached form a 3-10 membered heterocycle ring which optionally contains one to three heteroatom or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents. R₄, and R₅ are each independently selected from the group consisting of hydrogen, alkyl, cycloalkyl, alkoxyalkyl, heterocycloalkyl, hydroxyalkyl, and (NR_{A}R_{B})alkyl; Y is selected from the group consisting of:

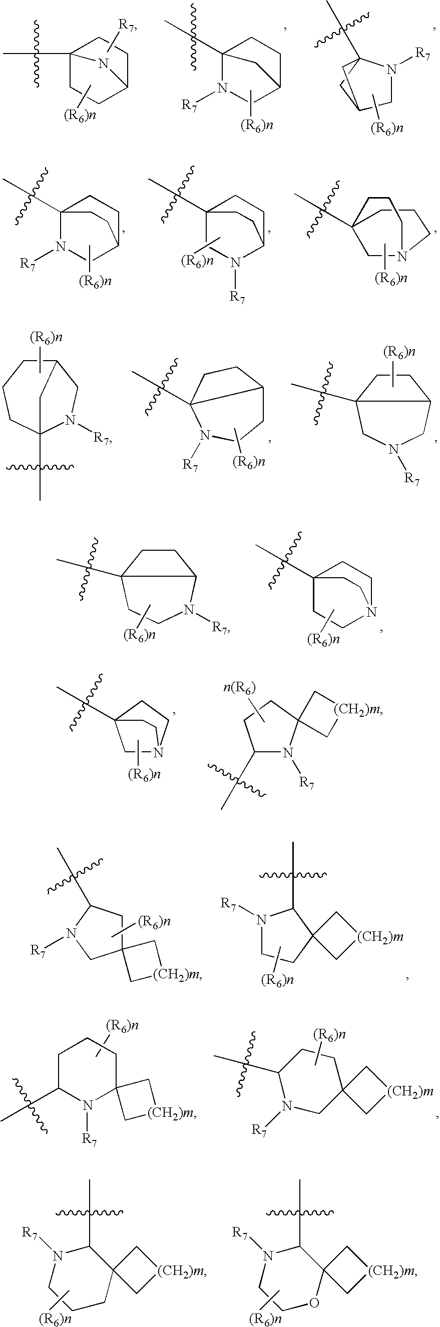

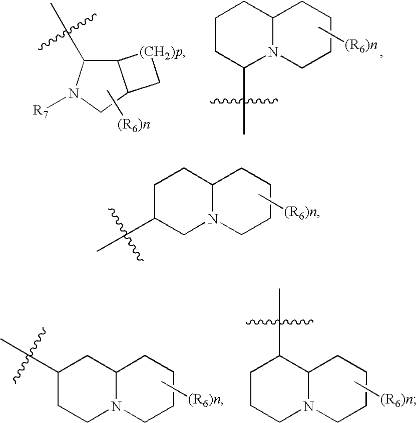

n is 0, 1, 2 or 3; m is 0, 1, 2 or 3; p is 0, 1, 2 or 3; R₆ is selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, hydroxyl, hydroxyalkyl, nitro, oxo, heteroaryl, heteroarylalkoxy, heteroaryloxy, heteroarylthio, heteroarylalkylthio, heterocycloalkyl, heterocycloalkoxy, heterocycloalkylthio, heterocyclooxy, heterocyclothio, NR_{A}R_{B}, (NR_{A}R_{B})alkyl, (NR_{A}R_{B})carbonyl, (NR_{A}R_{B})carbonylalkyl, and (NR_{A}R_{B})sulfonyl and is optionally attached to either one or both of the cyclic rings; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein R₁, R₂, and R₃, are hydrogen; R₄, and R₅ are each independently selected from the group consisting of hydrogen, alkyl, cycloalkyl, alkoxyalkyl, heterocycloalkyl, hydroxyalkyl, and (NR_{A}R_{B})alkyl; Y is selected from the group consisting of:

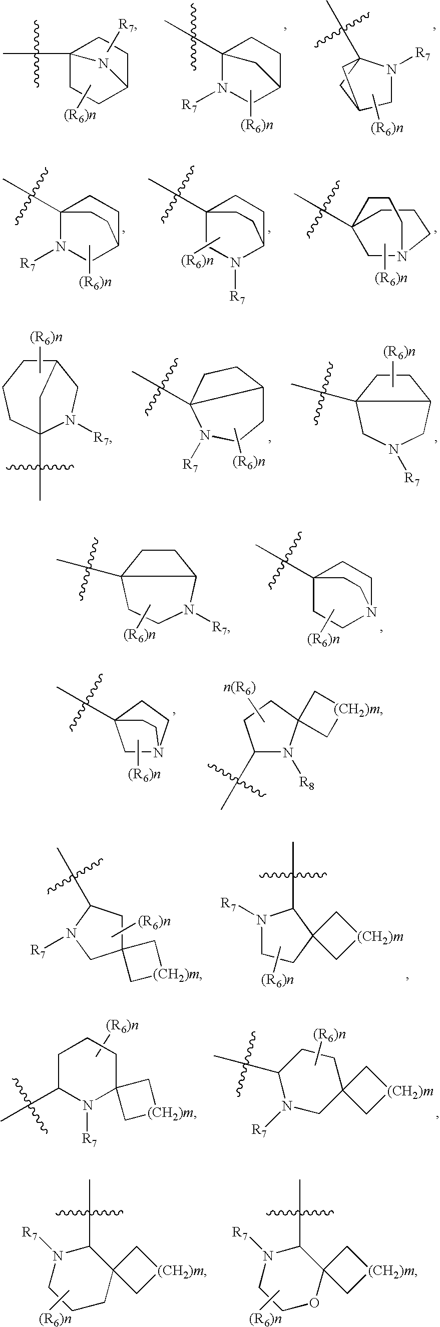

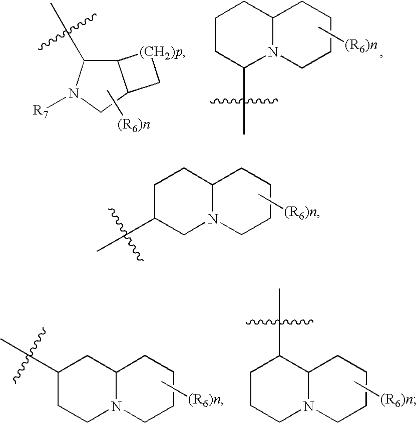

n is 0, 1, 2 or 3; m is 0, 1, 2 or 3; p is 0, 1, 2 or 3; R₆ is selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, hydroxyl, hydroxyalkyl, nitro, oxo, heteroaryl, heteroarylalkoxy, heteroaryloxy, heteroarylthio, heteroarylalkylthio, heterocycloalkyl, heterocycloalkoxy, heterocycloalkylthio, heterocyclooxy, heterocyclothio, NR_{A}R_{B}, (NR_{A}R_{B})alkyl, (NR_{A}R_{B})carbonyl, (NR_{A}R_{B})carbonylalkyl, and (NR_{A}R_{B})sulfonyl and is optionally attached to either one or both of the cyclic rings; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, and (NR_{A}R_{B})sulfonyl; (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein R₁, R₂, R₃, R₄, and R₅, are hydrogen; Y is selected from the group consisting of:

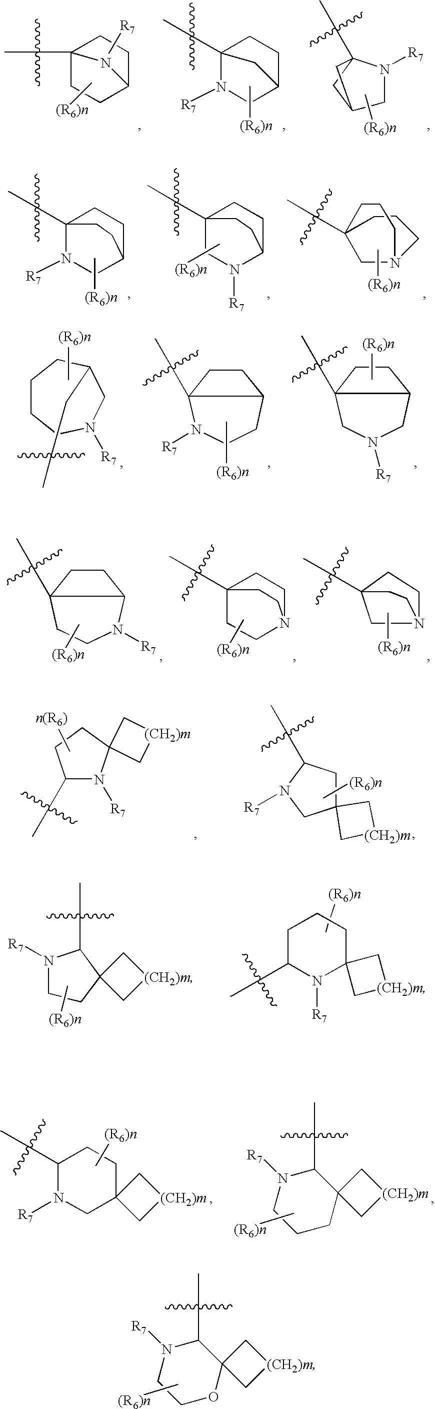

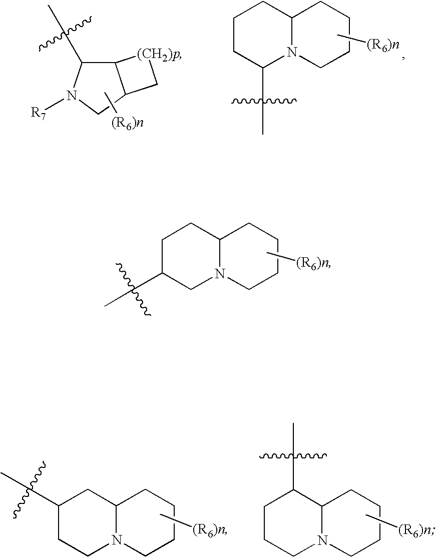

n is 0,1,2 or 3; m is 0, 1, 2 or 3; p is 0, 1, 2 or 3; R₆ is selected from the group consisting of a alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, hydroxyl, hydroxyalkyl, nitro, oxo, heteroaryl, heteroarylalkoxy, heteroaryloxy, heteroarylthio, heteroarylalkylthio, heterocycloalkyl, heterocycloalkoxy, heterocycloalkylthio, heterocyclooxy, heterocyclothio, NR_{A}R_{B}, (NR_{A}R_{B})alkyl, (NR_{A}R_{B})carbonyl, (NR_{A}R_{B})carbonylalkyl, and (NR_{A}R_{B})sulfonyl and in some embodiments is attached to either one or both of the cyclic rings; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein R₁, R₂, R₃, R₄, and R₅, are hydrogen; Y is selected from the group consisting of:

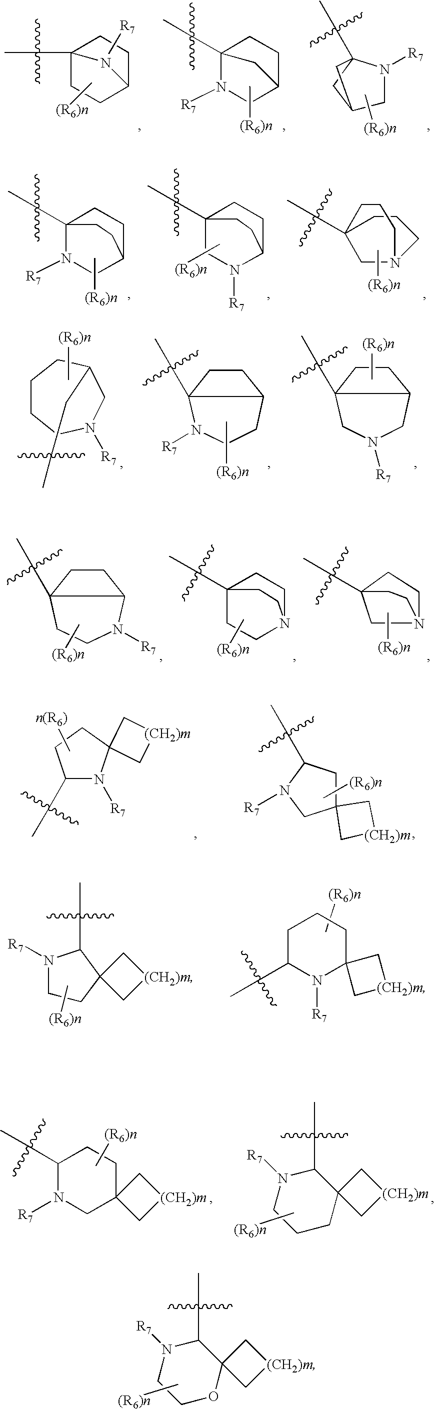

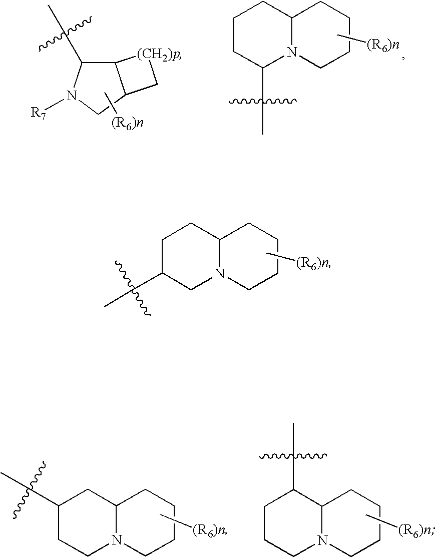

n is 0; m is 0, 1, 2 or 3; p is 0, 1, 2 or 3; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

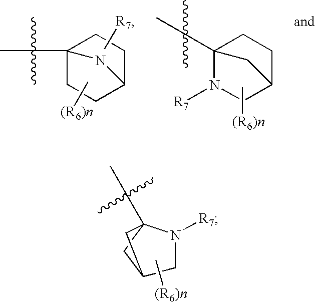

and n, R₁, R₂, R₃, R₄, R₅, R₆, and R₇ are as defined in Formula (I).

In another embodiment are provided compounds of Formula (I) or a therapeutically acceptable salt thereof wherein Y is selected from the group consisting of:

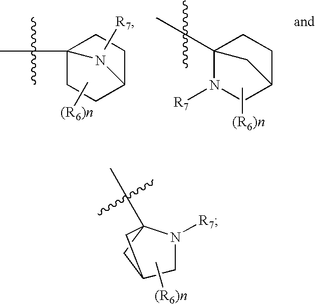

and n is 0; R₁, R₂, R₃, R₄, and R₅, are hydrogen; R₇ is selected from the group consisting of hydrogen, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

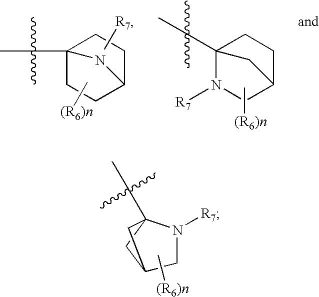

and n is 0; R₁, R₂, R₃, R₄, and R₅, are hydrogen; R₇ is selected from the group consisting of hydrogen, alkyl, cycloalkyl, arylalkyl, and (NR_{A}R_{B})sulfonyl; and R_{A}, and R_{B} are independently selected from the group consisting of hydrogen, alkyl, and cycloalkyl.

In another embodiment are provided compounds of Formula (I) or a therapeutically acceptable salt thereof wherein Y is selected from the group consisting of:

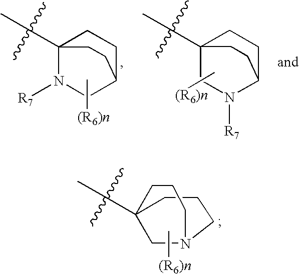

and n, R₁, R₂, R₃, R₄, R₅, R₆, and R₇ are as defined in Formula (I).

In another embodiment are provided compounds of Formula (I) or a therapeutically acceptable salt thereof wherein Y is selected from the group consisting of:

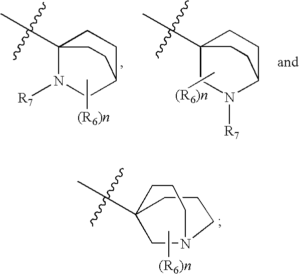

and n is 0; R₁, R₂, R₃, R₄, and R₅, are hydrogen; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

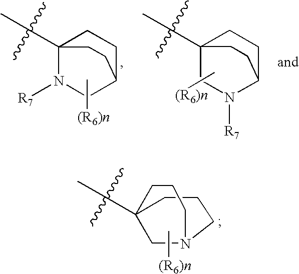

and n is 0; R₁, R₂, R₃, R₄, and R₅, are hydrogen; R₇ is selected from the group consisting of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

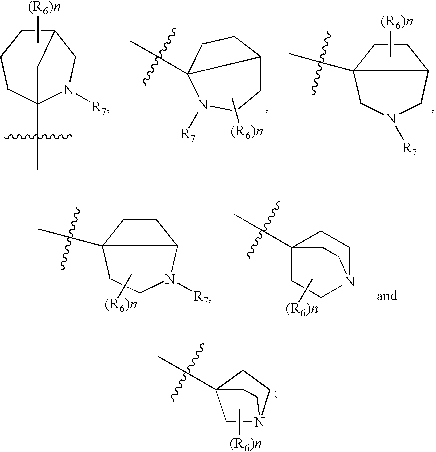

and n, R₁, R₂, R₃, R₄, R₅, R₆, and R₇ are as defined in Formula (I).

In another embodiment are provided compounds of Formula (I) or a therapeutically acceptable salt thereof wherein Y is selected from the group consisting of:

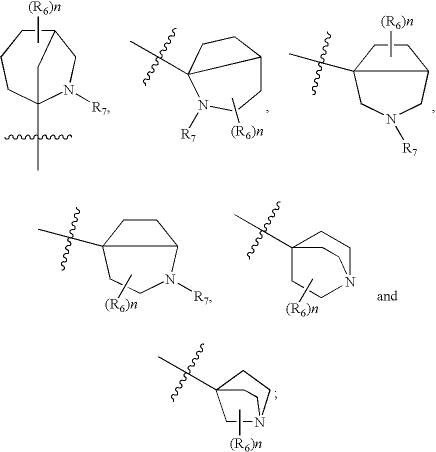

and n is 0; R₁, R₂, R₃, R₄, and R₅, are hydrogen; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, (NR_{A}R_{B})alkyl, (NR_{A}R_{B})carbonyl, (NR_{A}R_{B})carbonylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

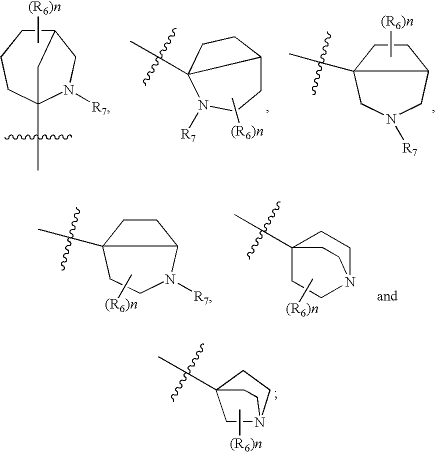

and n is 0; R₁, R₂, R₃, R₄, and R₅, are hydrogen; R₇ is selected from the group consisting of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

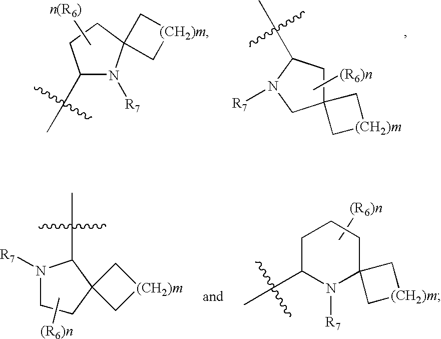

and n, m, R₁, R₂, R₃, R₄, R₅, R₆, and R₇ are as defined in Formula (I).

In another embodiment are provided compounds of Formula (I) or a therapeutically acceptable salt thereof wherein Y is selected from the group consisting of:

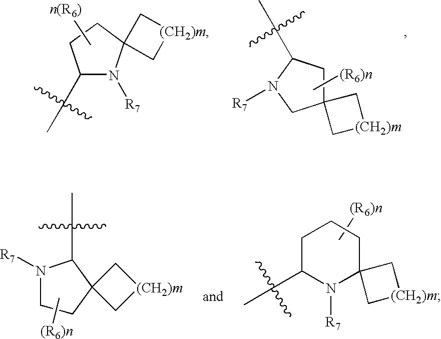

and n is 0; m is 0, 1, 2 or 3; R₁, R₂, R₃, R₄, and R₅, are hydrogen; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, (NR_{A}R_{B})alkyl, alkylcarbonyl, arylcarbonyl, heteroarylcarbonyl alkylsulfonyl, arylsulfonyl, heteroarylsulfonyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

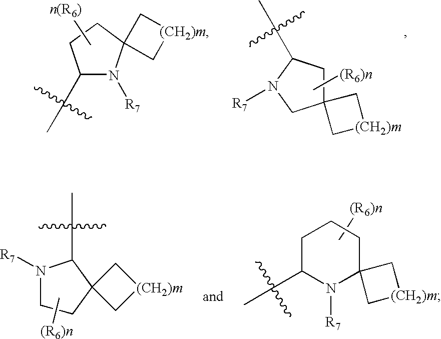

and n is 0; m is 0, 1, 2, or 3; R₁, R₂, R₃, R₄, and R₅, are hydrogen; R₇ is selected from the group consisting of hydrogen, alkyl, cycloalkyl, arylalkyl, and (NR_{A}R_{B})sulfonyl; and R_{A}, and R_{B} are independently selected from the group consisting of hydrogen, and alkyl.

In another embodiment are provided compounds of Formula (I) or a therapeutically acceptable salt thereof wherein Y is selected from the group consisting of:

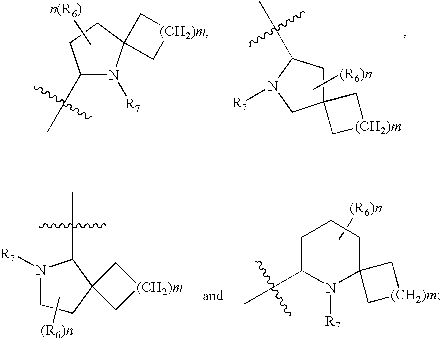

and n is 0; m is 0 or 1; R₁, R₂, R₃, R₄, and R₅, are hydrogen; R₇ is selected from the group consisting of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

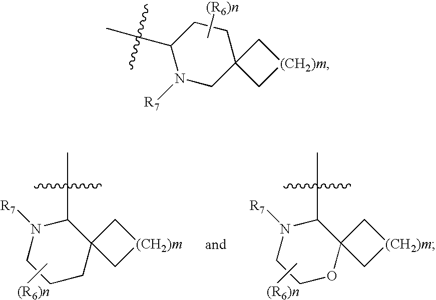

and n, m, R₁, R₂, R₃, R₄, R₅, R₆, and R₇ are as defined in Formula (I).

In another embodiment are provided compounds of Formula (I) or a therapeutically acceptable salt thereof wherein Y is selected from the group consisting of:

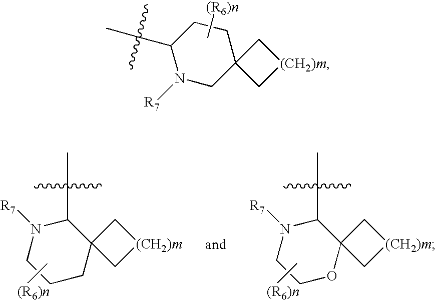

and n is 0; m is 0, 1, 2 or 3; R₁, R₂, R₃, R₄, and R₅, are hydrogen; R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

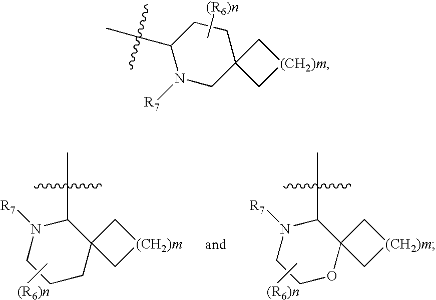

and n is 0; m is 0, 1, 2, or 3; R₁, R₂, R₃, R₄, and R₅, are hydrogen; R₇ is selected from the group consisting of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

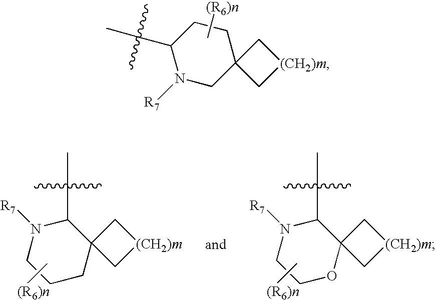

and n is 0; m is 0 or 1; R₁, R₂, R₃, R₄, and R₅, are hydrogen; R₇ is selected from the group consistin of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

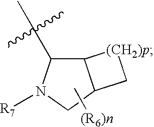

and n, p, R₁, R₂, R₃, R₄, R₅, R₆, and R₇ are as defined in Formula (I).

In another embodiment are provided compounds of Formula (I) or a therapeutically acceptable salt thereof wherein Y is selected from the group consisting of:

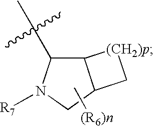

and n is 0; p is 0, 1, 2 or 3; R₁, R₂, R₃, R₄, and R₅, are hydrogen; R₇is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, (NR_{A}R_{B})alkyl, (NR_{A}R_{B})carbonyl, alkylcarbonyl, arylcarbonyl, heteroarylcarbonyl alkylsulfonyl, arylsulfonyl, heteroarylsulfonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

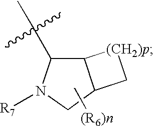

and n is 0; p is 0, 1, 2, or 3; R₁, R₂, R₃, R₄, and R₅, are hydrogen; R₇ is selected from the group consisting of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

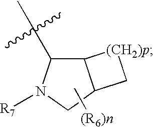

and n is 0; p is 0 or 1; R₁, R₂, R₃, R₄, and R₅, are hydrogen; R₇ is selected from the group consisting of hydrogen, alkyl, cycloalkyl, arylalkyl, alkylcarbonyl, arylcarbonyl, heteroarylcarbonyl alkylsulfonyl, arylsulfonyl, heteroarylsulfonyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

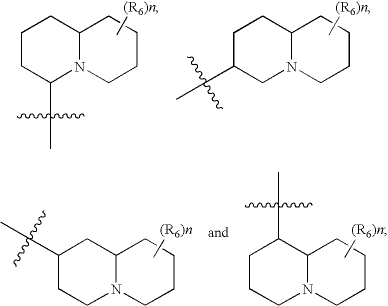

and n, R₁, R₂, R₃, R₄, R₅, and R₆ are as defined in Formula (I).

In another embodiment are provided compounds of Formula (I) or a therapeutically acceptable salt thereof wherein Y is selected from the group consisting of:

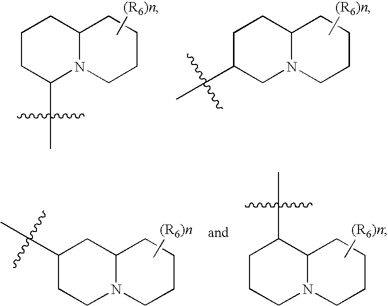

and n is 0, 1, 2 or 3; R₁, R₂, R₃, R₄, and R₅, are hydrogen; R₆ is selected from the group consistomg pf alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, hydroxyl, hydroxyalkyl, nitro, oxo, heteroaryl, heteroarylalkoxy, heteroaryloxy, heteroarylthio, heteroarylalkylthio, heterocycloalkyl, heterocycloalkoxy, heterocycloalkylthio, heterocyclooxy, heterocyclothio, NR_{A}R_{B}, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl; and R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents.

In another embodiment are provided compounds of Formula (I) or a therapeutically acceptable salt thereof wherein Y is selected from the group consisting of:

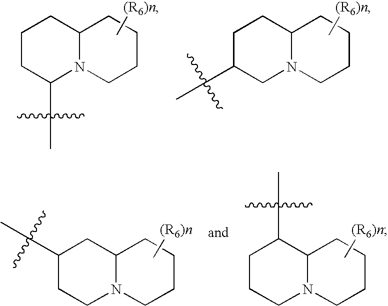

and n is 0; R₁, R₂, R₃, R₄, and R₅, are hydrogen.

In some embodiments, provided herein is a pharmaceutical composition comprising of a compound of Formula (I), or a pharmaceutically acceptable salt, a pharmaceutically acceptable solvate, pharmaceutically acceptable prodrug thereof and a pharmaceutically acceptable carrier, excipient, binder or diluent.

Another embodiment provides a method of inhibiting PARP in a subject in recognized need of such treatment comprising administering to the subject a therapeutically acceptable amount of a compound of Formula (I) or a therapeutically acceptable salt thereof.

In one embodiment, provided herein is a method of treatment of disease ameliorated by the inhibition of PARP that includes administering to a subject in need of treatment a therapeutically-effective amount of a compound of Formula (I). In some embodiments, the disease is selected from the group consisting of: vascular disease; septic shock; ischemic injury; reperfusion injury; neurotoxicity; hemorrhagic shock; inflammatory diseases; multiple sclerosis; secondary effects of diabetes; and acute treatment of cytotoxicity following cardiovascular surgery.

In certain embodiments, provided herein is a method for the treatment of cancer, which includes administering to a subject in need of treatment a therapeutically-effective amount of a compound of Formula (I).

Another embodiment provides a method of potentiation of cytotoxic cancer therapy in a subject in recognized need of such treatment comprising administering to the subject a therapeutically acceptable amount of a compound of Formula (I) or a therapeutically acceptable salt thereof.

In some embodiments, provided herein is a method for the treatment of cancer, which includes administering to a subject in need of treatment a therapeutically-effective amount of a compound of Formula (I) in combination with ionizing radiation or one or more chemotherapeutic agents. In some embodiments, the compound described herein is administered simultaneously with ionizing radiation or one or more chemotherapeutic agents. In other embodiments, the compound described herein is administered sequentially with ionizing radiation or one or more chemotherapeutic agents.

In certain embodiments, provided herein is a method for the treatment of cancer, which includes administering to a subject in need of treatment a therapeutically-effective amount of a compound of Formula (I) in combination with ionizing radiation and one or more chemotherapeutic agents. In some embodiments, the compound described herein is administered simultaneously with ionizing radiation and one or more chemotherapeutic agents. In other embodiments, the compound described herein is administered sequentially with ionizing radiation and one or more chemotherapeutic agents.

Another embodiment provides a method of treating leukemia, colon cancer, glioblastomas, lymphomas, melanomas, carcinomas of breast, or cervical carcinomas in a subject in recognized need of such treatment comprising administering to the subject a therapeutically acceptable amount of a compound of Formula (I) or therapeutically acceptable salt thereof.

In some embodiments, provided herein is a method of treatment of a cancer deficient in Homologous Recombination (HR) dependent DNA double strand break (DSB) repair pathway, which includes administering to a subject in need of treatment a therapeutically-effective amount of a compound of Formula (I). In certain embodiments, the cancer includes one or more cancer cells having a reduced or abrogated ability to repair DNA DSB by HR relative to normal cells. In other embodiments, the cancer cells have a BRCA1 or BRCA2 deficient phenotype. In some embodiments, the cancer cells are deficient in BRCA1 or BRCA2. In other embodiments, the methods provided herein involve treatment of an individual who is heterozygous for a mutation in a gene encoding a component of the HR dependent DNA DSB repair pathway. In another embodiment, the individual is heterozygous for a mutation in BRCA1 and/or BRCA2. In some embodiments, the method of treatment of a cancer includes treatment of breast, ovary, pancreas and/or prostate cancer. In some embodiments, the method of treatment of a cancer further includes administration of ionizing radiation or a chemotherapeutic agent.

Another embodiment provides a method of treating ischemia reperfusion injury associated with, but not limited to, myocardial infarction, stroke, other neural trauma, and organ transplantation, in a subject in recognized need of such treatment comprising administering to the subject a therapeutically acceptable amount of a compound of Formula (I) or a therapeutically acceptable salt thereof.

Another embodiment provides a method of treating reperfusion including, but not limited to, reperfusion of the eye, kidney, gut, and skeletal muscle, in a subject in recognized need of such treatment comprising administering to the subject a therapeutically acceptable amount of a compound of Formula (I) or a therapeutically acceptable salt thereof.

Another embodiment provides a method of treating inflammatory diseases including, but not limited to, arthritis, gout, inflammatory bowel disease, CNS inflammation, multiple sclerosis, allergic encephalitis, sepsis, septic shock, hemmorhagic shock, pulmonary fibrosis, and uveitis in a subject in recognized need of such treatment comprising administering to the subject a therapeutically acceptable amount of a compound of Formula (I) or a therapeutically acceptable salt thereof.

Another embodiment provides a method of treating immunological diseases or disorders such a rheumatoid arthritis and septic shock in a subject in recognized need of such treatment comprising administering to the subject a therapeutically acceptable amount of a compound of Formula (I) or a therapeutically acceptable salt thereof.

Another embodiment provides a method of treating degenerative diseases including, but not limited to, diabetes and Parkinsons disease in a subject in recognized need of such treatment comprising administering to the subject a therapeutically acceptable amount of a compound of Formula (I) or a therapeutically acceptable salt thereof.

Another embodiment provides a method of treating hypoglycemia in a subject in recognized need of such treatment comprising administering to the subject a therapeutically acceptable amount of a compound of Formula (I) or a therapeutically acceptable salt thereof.

Another embodiment provides a method of treating retroviral infection in a subject in recognized need of such treatment comprising administering to the subject a therapeutically acceptable amount of a compound of Formula (I) or a therapeutically acceptable salt thereof.

Another embodiment provides a method of treating liver toxicity following acetaminophen overdose in a subject in recognized need of such treatment comprising administering to the subject a therapeutically acceptable amount of a compound of Formula (I) or a therapeutically acceptable salt thereof.

Another embodiment provides a method of treating cardiac and kidney toxicities from doxorubicin and platinum based antineoplastic agents in a subject in recognized need of such treatment comprising administering to the subject a therapeutically acceptable amount of a compound of Formula (I) or a therapeutically acceptable salt thereof.

Another embodiment provides a method of treating skin damage secondary to sulfur mustards in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for inhibiting the PARP enzyme in a subject in recognized need of such treatment.

Another embodiment provides a use of a compound of Formula (I) or a therapeutically acceptable salt thereof, to prepare a medicament for inhibiting tumor growth in a subject in recognized need of such treatment.

Another embodiment provides a use of a compound of Formula (I) or a therapeutically acceptable salt thereof, to prepare a medicament for treating cancer in a subject in recognized need of such treatment.

Another embodiment provides a use of a compound of Formula (I) or a therapeutically acceptable salt thereof, to prepare a medicament for treating leukemia, colon cancer, glioblastomas, lymphomas in a subject in recognized need of such treatment.

Another embodiment provides a method of treating degenerative diseases including, but not limited to, diabetes and Parkinsons disease in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for potentiation of cytotoxic cancer therapy in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating ischemia reperfusion injury associated with, but not limited to, myocardial infarction, stroke, other neural trauma, and organ transplantation, in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating reperfusion including, but not limited to, reperfusion of the eye, kidney, gut and skeletal muscle, in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating inflammatory diseases including, but not limited to, arthritis, gout, inflammatory bowel disease, CNS inflammation, multiple sclerosis, allergic encephalitis, sepsis, septic shock, hemmorhagic shock, pulmonary fibrosis, and uveitis in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating immunological diseases or disorders such as rheumatoid arthritis and septic shock in a mammal in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating hypoglycemia in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating retroviral infection in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating liver toxicity following acetaminophen overdose in a subject in recognized need of such treatment comprising administering to the subject a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating cardiac and kidney toxicities from doxorubicin and platinum based antineoplastic agents in a subject in recognized need of such treatment comprising administering to the mammal a therapeutically acceptable amount of a compound of Formula (I) or a therapeutically acceptable salt thereof.

Another embodiment provides a use of a compound of Formula (I) or a therapeutically acceptable salt thereof, to prepare a medicament for treating skin damage secondary to sulfur mustards in a subject in recognized need of such treatment comprising administering to the mammal a therapeutically acceptable amount of a compound of Formula (I) or a therapeutically acceptable salt thereof.

Articles of manufacture, which include packaging material, a compound provided herein that is effective for modulating the activity of the enzyme poly(ADP-ribose)polymerase, or for treatment, prevention or amelioration of one or more symptoms of a poly(ADP-ribose)polymerase-dependent or poly(ADP-ribose)polymerase-mediated disease or condition, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for modulating the activity of poly(ADP-ribose)polymerase, or for treatment, prevention or amelioration of one or more symptoms of a poly(ADP-ribose)polymerase-dependent or poly(ADP-ribose)polymerase-mediated disease or condition, are provided.

Any combination of the groups described above for the various variables is contemplated herein.

In one embodiment, disclosed herein is a pharmaceutical composition that includes a compound, pharmaceutically acceptable salt, pharmaceutically acceptable N-oxide, pharmaceutically active metabolite, pharmaceutically acceptable prodrug, or pharmaceutically acceptable solvate of any of the compounds disclosed herein. In another embodiment, the pharmaceutical compositions further include a pharmaceutically acceptable diluent, excipient or binder. In another embodiment, the pharmaceutical composition further includes a second pharmaceutically active ingredient.

In one embodiment, the PARP mediated disease or condition in a patient, or the PARP dependent disease or condition in a patient is cancer or a non-cancerous disorder. In another embodiment, the disease or condition is iatrogenic.

In some embodiments are methods for reducing/inhibiting the activity of PARP in a subject that include administering to the subject at least once an effective amount of a compound described herein.

Other embodiments are methods for modulating, including reducing and/or inhibiting the activity of PARP, directly or indirectly, in a subject that includes administering to the subject at least once an effective amount of at least one compound described herein.

In further embodiments are methods for treating PARP mediated conditions or diseases, that include administering to the subject at least once an effective amount of at least one compound described herein.

Other embodiments include the use of a compound described herein in the manufacture of a medicament for treating a disease or condition in a subject in which the activity of at least one PARP-protein contributes to the pathology and/or symptoms of the disease or condition.

In any of the aforementioned embodiments are further embodiments in which administration is enteral, parenteral, or both, and wherein:
(a) the effective amount of the compound is systemically administered to the subject;
(b) the effective amount of the compound is administered orally to the subject;
(c) the effective amount of the compound is intravenously administered to the subject;
(d) the effective amount of the compound administered by inhalation;
(e) the effective amount of the compound is administered by nasal administration;
(f) the effective amount of the compound is administered by injection to the subject;
(g) the effective amount of the compound is administered topically (dermal) to the subject;
(h) the effective amount of the compound is administered by ophthalmic administration; and/or
(i) the effective amount of the compound is administered rectally to the subject.

In any of the aforementioned embodiments are further embodiments that include single administrations of the effective amount of the compound, including further embodiments in which the compound is administered to the subject (i) once; (ii) multiple times over the span of one day; (iii) continually; or (iv) continuously.

In any of the aforementioned embodiments are further embodiments that include multiple administrations of the effective amount of the compound, including further embodiments wherein:

(i) the compound is administered in a single dose;

(ii) the time between multiple administrations is every 6 hours;

(iii) the compound is administered to the subject every 8 hours.

In further or alternative embodiments, the method includes a drug holiday, wherein the administration of the compound is temporarily suspended or the dose of the compound being administered is temporarily reduced; at the end of the drug holiday, dosing of the compound is resumed. In some embodiments, the length of the drug holiday varies from 2 days to 1 year.

In any of the aforementioned embodiments involving the treatment of proliferative disorders, including cancer, are further embodiments that include administering at least one additional agent selected from among alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, paclitaxel, Taxol , temozolomide, thioguanine, and classes of drugs including hormones (an antiestrogen, an antiandrogen, or gonadotropin releasing hormone analogues, interferons such as, for example, alpha interferon, nitrogen mustards such as, for example, busulfan, melphalan or mechlorethamine, retinoids such as, for example, tretinoin, topoisomerase inhibitors such as, for example, irinotecan or topotecan, tyrosine kinase inhibitors such as, for example, gefinitinib or imatinib, and agents to treat signs or symptoms induced by such therapy including allopurinol, filgrastim, granisetron/ondansetron/palonosetron, and dronabinol.

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent to those skilled in the art from this detailed description. All references cited herein, including patents, patent applications, and publications, are hereby incorporated by reference in their entirety.

Described herein are compounds, methods of making such compounds, pharmaceutical compositions and medicaments that include such compounds, and methods of using such compounds to treat or prevent diseases or conditions associated with PARP activity.

Described herein are compounds having activity in inhibiting the enzyme poly(ADP-ribose)polymerase (PARP). In some embodiments, the compounds have the structure set forth in Formula (I).

The mammalian enzyme PARP-1 is a multidomain protein. PARP-1 has been implicated in the signaling of DNA damage through its ability to recognize and rapidly bind to DNA single or double strand breaks (D'Amours, et al, Biochem. J., 342, 249-268 (1999); Virag et al. Pharmacological Reviews, vol. 54, no. 3, 375-429, 2002).

The family of poly(ADP-ribose)polymerases includes approximately 18 proteins, which all display a certain level of homology in their catalytic domain but differ in their cellular functions (Ame et al., BioEssays., 26(8), 882-893 (2004)). PARP-1 and PARP-2 are unique members of the family, in that their catalytic activities are stimulated by the occurrence of DNA strand breaks.

It is now known that PARP-1 participates in a variety of DNA-related functions including gene amplification, cell division, differentiation, apoptosis, DNA base excision repair as well as effects on telomere length and chromosome stability (d'Adda di Fagagna, et al, Nature Gen., 23(1), 76-80 (1999)).

Studies on the mechanism by which PARP-1 modulates DNA repair and other processes have identified its importance in the formation of poly(ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP-1 utilizes NAD+ to synthesize poly(ADP-ribose) on a variety of nuclear target proteins, including topoisomerases, histones and PARP itself (Rhun, et al., Biochem. Biophys. Res. Commun., 245, 1-10 (1998)).

Poly(ADP-ribosyl)ation has also been associated with malignant transformation. For example, PARP-1 activity is higher in the isolated nuclei of SV40-transformed fibroblasts, while both leukemic cells and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Biol. Med., 149, 933-938 (1975); and Hirai, et al., Cancer Res., 43, 3441-3446 (1983)). It has also been demonstrated that malignant prostate tumours have increased levels of active PARP as compared to benign prostate cells, which is associated with higher levels of genetic instability (Mcnealy, et al., Anticancer Res., 23, 1473-1478 (2003)).

In cells treated with alkylating agents, the inhibition of PARP leads to a marked increase in DNA-strand breakage and cell killing. PARP-1 inhibitors also enhance the effects of radiation response by suppressing the repair of potentially lethal damage. PARP inhibitors are also effective in radio-sensitizing hypoxic tumor cells. In certain tumor cell lines, chemical inhibition of PARP activity is also associated with marked sensitization to very low doses of radiation.

Furthermore, PARP-1 knockout (PARP −) animals exhibit genomic instability in response to alkylating agents and y-irradiation (Wang, et al., Genes Dev., 9, 509-520 (1995); Menissier de Murcia, et al., Proc. Natl Acad. Sci. USA, 94, 7303-7307 (1997)). More recent data indicates that PARP-1 and PARP-2 possess both overlapping and non-redundant functions in the maintenance of genomic stability, making them both interesting targets (Menissier de Murcia, et al., EMBO. J., 22(9), 2255-2263 (2003)).

A role for PARP-1 has also been demonstrated in certain vascular diseases, such as, for example, septic shock, ischaemic injury and neurotoxicity (Cantoni, et al., Biochim. Biophys. Acta, 1014, 1-7 (1989); Szabo, et al., J. Clin. Invest., 100, 723-735 (1997)). Oxygen radical DNA damage that leads to strand breaks in DNA, which are subsequently recognized by PARP-1, is a major contributing factor to such disease states as shown by PARP-1 inhibitor studies (Cosi, et al., J. Neurosci. Res., 39, 3846 (1994); Said, et al., Proc. Natl Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)).

It has also been demonstrated that efficient retroviral infection of mammalian cells is blocked by the inhibition of PARP-1 activity. Such inhibition of recombinant retroviral vector infections was shown to occur in various different cell types (Gaken, et al., J. Virology, 70(6), 3992-4000 (1996)). Inhibitors of PARP-1 have been developed for the use in anti-viral therapies and in cancer treatment.

Moreover, PARP-1 inhibition has been speculated to delay the onset of aging characteristics in human fibroblasts (Rattan and Clark, Biochem. Biophys. Res. Comm., 201(2), 665-672 (1994)). This may be related to the role that PARP plays in controlling telomere function (d'Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H. B., et al., Am. J. Physiol. Gastrointest. Liver Physiol., 279, G641-G651 (2000).

In some embodiments, PARP inhibitors, such as those of Formula (I), find utility in: (a) preventing poly(ADP-ribose) chain formation by inhibiting the activity of cellular PARP (PARP-1 and/or PARP-2); (b) the treatment of: vascular disease; septic shock; ischaemic injury, both cerebral and cardiovascular; reperfusion injury, both cerebral and cardiovascular; neurotoxicity, including acute and chronic treatments for stroke and Parkinsons disease; haemorraghic shock; inflammatory diseases, such as arthritis, inflammatory bowel disease, ulcerative colitis and Crohn's disease; multiple sclerosis; secondary effects of diabetes; as well as the acute treatment of cytotoxicity following cardiovascular surgery or diseases ameliorated by the inhibition of the activity of PARP; (c) use as an adjunct in cancer therapy or for potentiating tumor cells for treatment with ionizing radiation or chemotherapeutic agents.

In particular, compounds provided herein, such as, for example, in some embodiments, Formula (I) is used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the method of administration for the particular agent.

PARP inhibitors, such as, for example, in other embodiments, compounds of Formula (I), are in the treatment of disease ameliorated by the inhibition of PARP, which includes administering to a subject in need of treatment a therapeutically-effective amount of a compound provided herein, and in one embodiment in the form of a pharmaceutical composition. PARP inhibitors, such as, for example, in further embodiments, compounds of Formula (I), are used in the treatment of cancer, which includes administering to a subject in need of treatment a therapeutically-effective amount of a compound provided herein in combination, and in one embodiment in the form of a pharmaceutical composition, simultaneously or sequentially with radiotherapy (ionizing radiation) or chemotherapeutic agents.

In some embodiments, PARP inhibitors, such as, for example, compounds of Formula (I), are used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, which includes administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 NM_{—}002877), RAD51C (NM_{—}002876), RAD51L3 (NM_002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY (Hughes-Davies, et al, Cell, 115, pp 523-535). HR components are also described in Wood, et al., Science, 291, 1284-1289 (2001).

A cancer which is deficient in HR dependent DNA DSB repair may include one or more cancer cells which have a reduced or abrogated ability to repair DNA DSBs through that pathway, relative to normal cells, i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway (see for example, Wood, et al., Science, 291, 1284-1289 (2001)) include the components listed above.

In some embodiments, the cancer cells have a BRCA1 and/or a BRCA2 deficient phenotype, i.e., BRCA1 and/or BRCA2 activity is reduced or abolished in the cancer cells. Cancer cells with this phenotype may be deficient in BRCA1 and/or BRCA2, i.e., expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol Med., 8(12), 571-6, (2002)). The association of BRCA1 and/or BRCA2 mutations with breast cancer has been described(Radice, P. J., Exp Clin Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some embodiments, the individual is heterozygous for one or more variations, such as mutations and polymorphisms, in BRCA1 and/or BRCA2 or a regulator thereof. The detection of variation in BRCA1 and BRCA2 is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

### Definitions

Unless defined otherwise, all technical and scientific terms used herein have the standard meaning pertaining to the claimed subject matter belongs. All patents, patent applications, published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information is found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Unless otherwise indicated, conventional methods of mass spectroscopy, NMR, HPLC, protein chemistry, biochemistry, recombinant DNA techniques and pharmacology are employed. Unless specific definitions are provided, the standard nomenclature employed in connection with, and the standard laboratory procedures and techniques of, analytical chemistry, synthetic organic chemistry, and medicinal and pharmaceutical chemistry are employed. In some embodiments, standard techniques are used for chemical syntheses, chemical analyses, pharmaceutical preparation, formulation, and delivery, and treatment of patients. In some embodiments, standard techniques are used for recombinant DNA, oligonucleotide synthesis, and tissue culture and transformation (e.g., electroporation, lipofection). In some embodiments, reactions and purification techniques are performed e.g., using kits of manufacturer's specifications or as commonly accomplished or as described herein. In other embodiments, the foregoing techniques and procedures are generally performed of conventional methods and as described in various general and more specific references that are cited and discussed throughout the present specification.

As used throughout this application and the appended claims, the following terms have the following meanings:

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. In some embodiments, depending on the structure, an alkenyl group is a monoradical or a diradical (i.e., an alkenylene group). In some embodiments, alkenyl groups are optionally substituted. Illustrative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-cecenyl.

The term “alkoxy” as used herein, means an alkyl group, as defined herein, appended to the parent molecular moiety through an oxygen atom. Illustrative examples of alkoxy include, but are not limited to, methoxy, ethoxy, propoxy, 2-propoxy, butoxy, tert-butoxy, pentyloxy, and hexyloxy.

The term “alkyl” as used herein, means a straight, branched chain, or cyclic (in this case, it would also be known as “cycloalkyl”) hydrocarbon containing from 1-10 carbon atoms. Illustrative examples of alkyl include, but are not limited to, methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

The term “cycloalkyl” as used herein, means a monocyclic or polycyclic radical that contains only carbon and hydrogen, and in some embodiments are saturated, partially unsaturated, or fully unsaturated. Cycloalkyl groups include groups having from 3 to 10 ring atoms. Illustrative examples of cyclic include but are not limited to, the following moieties:

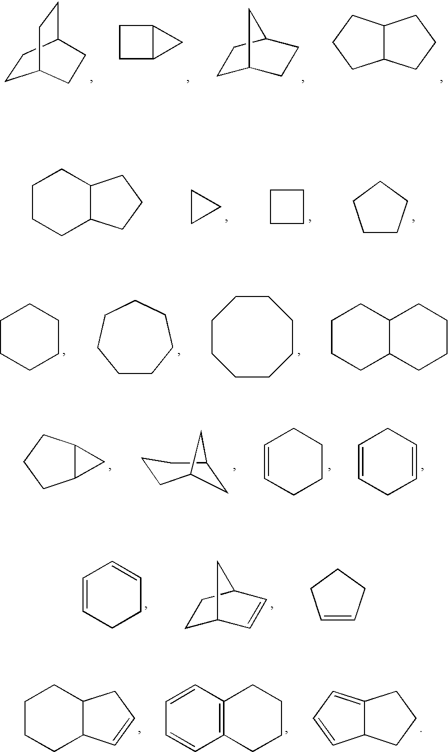

In some embodiments, depending on the structure, a cycloalkyl group is a monoradical or a diradical (e.g., a cycloalkylene group).

The term “cycloalkyl groups” as used herein refers to groups which are optionally substituted with 1, 2, 3, or 4 substituents selected from alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxyl, hydroxyalkyl, mercapto, oxo, —NR_{A}R_{A}, and (NR_{A}R_{B})carbonyl.

The term “cycloalkylalkyl” as used herein, means a cycloalkyl group, as defined herein, appended to the parent molecular moiety through an alkyl group, as defined herein. Illustrative examples of cycloalkylalkyl include, but are not limited to, cyclopropylmethyl, 2-cyclobutylethyl, cyclopentylmethyl, cyclohexylmethyl, and 4-cycloheptylbutyl.

The term “carbocyclic” as used herein, refers to a compound which contains one or more covalently closed ring structures, and that the atoms forming the backbone of the ring are all carbon atoms

The term “carbocycle” as used herein, refers to a ring, wherein each of the atoms forming the ring is a carbon atom. In some embodiments, carbocyclic rings are formed by three, four, five, six, seven, eight, nine, or more than nine carbon atoms. In some embodiments, carbocycles are optionally substituted.

The term “alkoxyalkyl” as used herein, means at least one alkoxy group, as defined herein, appended to the parent molecular moiety through an alkyl group, as defined herein. Illustrative examples of alkoxyalkyl include, but are not limited to, 2-methoxyethyl, 2-ethoxyethyl, tert-butoxyethyl and methoxymethyl.

The term “alkoxycarbonyl” as used herein, means an alkoxy group, as defined herein, appended to the parent molecular moiety through a carbonyl group, as defined herein. Illustrative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein.

The term “alkylcarbonyl” as used herein, means an alkyl group, as defined herein, appended to the parent molecular moiety through a carbonyl group, as defined herein. Illustrative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Illustrative examples of alkylcarbonyloxy include, but are not limited to, acetyloxy, ethylcarbonyloxy, and tert-butylcarbonyloxy.

The term “alkylthio” or “thioalkoxy” as used herein, means an alkyl group, as defined herein, appended to the parent molecular moiety through a sulfur atom. Illustrative examples of alkylthio include, but are not limited to, methylthio, ethylthio, butylthio, tert-butylthio, and hexylthio.

The term “alkylthioalkyl” as used herein, means an alkylthio group, as defined herein, appended to the parent molecular moiety through an alkyl group, as defined herein. Illustrative examples of alkylthioalkyl include, but are not limited to, methylthiomethyl, 2-(ethylthio)ethyl, butylthiomethyl, and hexylthioethyl.

The term “alkynyl” as used herein, means a straight, branched chain hydrocarbon containing from 2-10 carbons and containing at least one carbon-carbon triple bond. In some embodiments, alkynyl groups are optionally substituted. Illustrative examples of alkynyl include, but are not limited to, acetylenyl, 1-propynyl, 2-propynyl, 3-butynyl, 2-pentynyl, and 1-butynyl.

The term “aromatic” as used herein, refers to a planar ring having a delocalized π-electron system containing 4n+2π electrons, where n is an integer. In some embodiments, aromatic rings are formed by five, six, seven, eight, nine, or more than nine atoms. In other embodiments, aromatics are optionally substituted. The term includes monocyclic or fused-ring polycyclic (i.e., rings which share adjacent pairs of carbon atoms) groups.

The term “aryl” as used herein, refers to an aromatic ring wherein each of the atoms forming the ring is a carbon atom. In some embodiments, aryl rings are formed by five, six, seven, eight, nine, or more than nine carbon atoms. Examples of aryl groups include, but are not limited to phenyl, naphthalenyl, phenanthrenyl, anthracenyl, fluorenyl, and indenyl.

In some embodiments, the term “aryl” as used herein means an aryl group that is optionally substituted with one, two, three, four or five substituents independently selected from the group consisting of alkenyl, alkoxy, alkoxyalkyl, alkoxycarbonyl, alkyl, alkylcarbonyl, alkylcarbonyloxy, alkylthio, alkylthioalkyl, alkynyl, carbonyl, cyano, formyl, haloalkoxy, haloalkyl, halogen, hydroxyl, hydroxyalkyl, mercapto, nitro, —NR_{A}R_{A}, and (NR_{A}R_{B})carbonyl.

The term “arylalkyl” as used herein, means an aryl group, as defined herein, appended to the parent molecular moiety through an alkyl group, as defined herein. Illustrative examples of arylalkyl include, but are not limited to benzyl, 2-phenylethyl, -phenylpropyl, 1-methyl-3-phenylpropyl, and 2-naphth-2-ylethyl.

The term “carbonyl” as used herein, means a —C(O)— group.

The term “carboxy” as used herein, means a —COOH group.

The term “cyano” as used herein, means a —CN group.

The term “formyl” as used herein, means a —C(O)H group.

The term “halo” or “halogen” as used herein, means a —Cl, —Br, —I or —F.

The term “mercapto” as used herein, means a —SH group.

The term “nitro” as used herein, means a —NO₂ group.

The term “hydroxy” as used herein, means a —OH group.

The term “oxo” as used herein, means a ═O group.

The term “bond” or “single bond” as used herein, refers to a chemical bond between two atoms, or two moieties when the atoms joined by the bond are considered to be part of larger substructure.

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “alkylamine” refers to the —N(alkyl)ₓH_{y} group, where x and y are selected from among x=1, y=1 and x=2, y=0. In some embodiments, when x=2, the alkyl groups, taken together with the N atom to which they are attached, optionally form a cyclic ring system.

The term “amide” as used herein, is a chemical moiety with the formula —C(O)NHR or —NHC(O)R, where R is selected from among hydrogen, alkyl, cycloalkyl, aryl, heteroaryl (bonded through a ring carbon) and heterocycloalkyl (bonded through a ring carbon). In some embodiments, an amide moiety forms a linkage between an amino acid or a peptide molecule and a compound described herein, thereby forming a prodrug. In some embodiments, any amine, or carboxyl side chain on the compounds described herein is amidified.

The term “ester” refers to a chemical moiety with formula —COOR, where R is selected from among alkyl, cycloalkyl, aryl, heteroaryl (bonded through a ring carbon) and heterocycloalkyl (bonded through a ring carbon). In some embodiments, any hydroxy, or carboxyl side chain on the compounds described herein is esterified.

The terms “heteroalkyl” “heteroalkenyl” and “heteroalkynyl” as used herein, include optionally substituted alkyl, alkenyl and alkynyl radicals in which one or more skeletal chain atoms are selected from an atom other than carbon, e.g., oxygen, nitrogen, sulfur, silicon, phosphorus or combinations thereof

The term “heteroatom” as used herein refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms are all the same as one another, or some or all of the two or more heteroatoms are each different from the others.

The term “ring” as used herein, refers to any covalently closed structure. Rings include, for example, carbocycles (e.g., aryls and cycloalkyls), heterocycles (e.g., heteroaryls and heterocycloalkyls), aromatics (e.g. aryls and heteroaryls), and non-aromatics (e.g., cycloalkyls and heterocycloalkyls). In some embodiments, rings are optionally substituted. In some embodiments, rings form part of a ring system.

As used herein, the term “ring system” refers to two or more rings, wherein two or more of the rings are fused. The term “fused” refers to structures in which two or more rings share one or more bonds.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In some embodiments, the polycyclic heteroaryl group is fused or non-fused. Illustrative of heteroaryl groups include, but are not limited to, the following moieties:

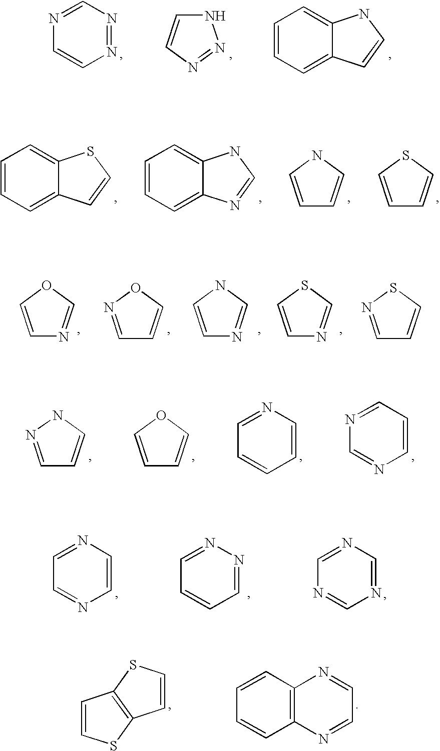

In some embodiments, depending on the structure, a heteroaryl group is a monoradical or a diradical (i.e., a heteroarylene group).

The term “heteroaryl” means heteroaryl groups that are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkyl, mercapto, nitro, —NR_{A}R_{B}, and —(NR_{A}R_{B})carbonyl.

The term “heteroarylalkyl” as used herein, means a heteroaryl, as defined herein, appended to the parent molecular moiety through an alkyl group, as defined herein. Illustrative examples of heteroarylalkyl include, but are not limited to, pyridinylmethyl.

The term “heterocycloalkyl” or “non-aromatic heterocycle” as used herein, refers to a non-aromatic ring wherein one or more atoms forming the ring is a heteroatom. A “heterocycloalkyl” or “non-aromatic heterocycle” group refers to a cycloalkyl group that includes at least one heteroatom selected from nitrogen, oxygen and sulfur. In some embodiments, the radicals are fused with an aryl or heteroaryl. In some embodiments, heterocycloalkyl rings are formed by three, four, five, six, seven, eight, nine, or more than nine atoms. In some embodiments, heterocycloalkyl rings are optionally substituted. In certain embodiments, heterocycloalkyls contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1 ,4-thiazine, 2H-1 ,2-oxazine , maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include, but are not limited to

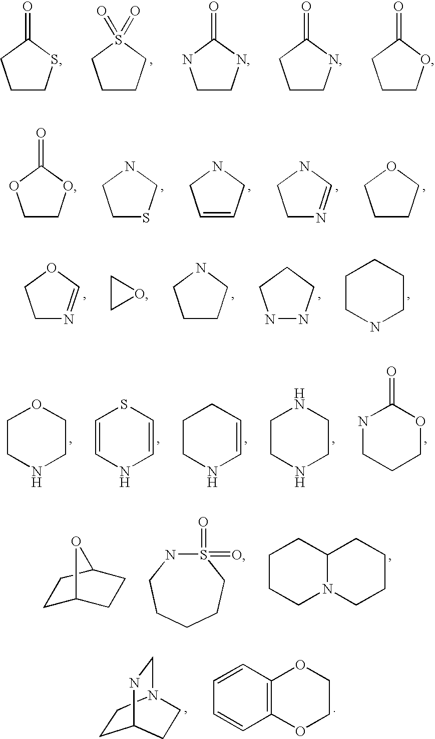

The term heterocycloalkyl also includes all ring forms of the carbohydrates, including but not limited to the monosaccharides, the disaccharides and the oligosaccharides.

The term “heterocycle” refers to heteroaryl and heterocycloalkyl used herein, refers to groups containing one to four heteroatoms each selected from O, S and N, wherein each heterocycle group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. In some embodiments, it is understood that the heterocycle ring has additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In some embodiments, in heterocycles that have two or more heteroatoms, those two or more heteroatoms are the same or different from one another. In some embodiments, heterocycles are optionally substituted. In some embodiments, binding to a heterocycle is at a heteroatom or via a carbon atom. Heterocycloalkyl groups include groups having only 4 atoms in their ring system, but heteroaryl groups must have at least 5 atoms in their ring system. The heterocycle groups include benzo-fused ring systems. An example of a 4-membered heterocycle group is azetidinyl (derived from azetidine). An example of a 5-membered heterocycle group is thiazolyl. An example of a 6-membered heterocycle group is pyridyl, and an example of a 10-membered heterocycle group is quinolinyl. Examples of heterocycloalkyl groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of heteroaryl groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. In some embodiments, the foregoing groups, as derived from the groups listed above, are C-attached or N-attached where such is possible. For instance, in some embodiments, a group derived from pyrrole is pyrrol-1-yl (N-attached) or pyrrol-3-yl (C-attached). Further, in some embodiments, a group derived from imidazole is imidazol-1-yl or imidazol-3-yl (both N-attached) or imidazol-2-yl, imidazol-4-yl or imidazol-5-yl (all C-attached). The heterocycle groups include benzo-fused ring systems and ring systems substituted with one or two oxo (=O) moieties such as pyrrolidin-2-one. In some embodiments, depending on the structure, a heterocycle group is a monoradical or a diradical (i.e., a heterocyclene group).

The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkyl, mercapto, nitro, —NR_{A}R_{B}, and —(NR_{A}R_{B})carbonyl.

The term “heterocycloalkoxy” refers to a heterocycloalkyl group, as defined herein, appended to the parent molecular moiety through an alkoxy group.

The term “heterocycloalkylthio” refers to a heterocycloalkyl group, as defined herein, appended to the parent molecular moiety through an alkylthio group.

The term “heterocyclooxy” refers to a heterocycloalkyl group, as defined herein, appended to the parent molecular moiety through an oxygen atom.

The term “heterocyclothio” refers to a heterocycloalkyl group, as defined herein, appended to the parent molecular moiety through a sulfur atom.

The term “heteroarylalkoxy” refers to a heteroaryl group, as defined herein, appended to the parent molecular moiety through an alkoxy group.

The term “heteroarylalkylthio” refers to a heteroaryl group, as defined herein, appended to the parent molecular moiety through an alkylthio group.

The term “heteroaryloxy” refers to a heteroaryl group, as defined herein, appended to the parent molecular moiety through an oxygen atom.

The term “heteroarylthio” refers to a heteroaryl group, as defined herein, appended to the parent molecular moiety through a sulfur atom.

In some embodiments, the term “membered ring” embraces any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “non-aromatic 5, 6, 7, 8, 9, 10, 11 or 12-bicyclic heterocycle” as used herein, means a heterocycloalkyl, as defined herein, consisting of two carbocyclic rings, fused together at the same carbon atom (forming a spiro structure) or different carbon atoms (in which two rings share one or more bonds), having 5 to 12 atoms in its overall ring system, wherein one or more atoms forming the ring is a heteroatom. Illustrative examples of non-aromatic 5, 6, 7, 8, 9, 10, 11, or 12-bicyclic heterocycle ring include, but are not limited to, 2-azabicyclo[2.2.1]heptanyl, 7-azabicyclo[2.2.1]heptanyl, 2-azabicyclo[3.2.0]heptanyl, 3-azabicyclo[3.2.0]heptanyl, 4-azaspiro[2.4]heptanyl, 5-azaspiro[2.4]heptanyl, 2-oxa-5-azabicyclo[2.2.1]heptanyl, 4-azaspiro[2.5]octanyl, 5-azaspiro[2.5]octanyl, 5-azaspiro[3.4]octanyl, 6-azaspiro[3.4]octanyl, 4-oxa-7-azaspiro[2.5]octanyl, 2-azabicyclo[2.2.2]octanyl, 1,3-diazabicyclo[2.2.2]octanyl, 5-azaspiro[3.5]nonanyl, 6-azaspiro[3.5]nonanyl, 5-oxo-8-azaspiro[3.5]nonanyl, octahydrocyclopenta[c]pyrrolyl, octahydro-1H-quinolizinyl, 2,3,4,6,7,9a-hexahydro-1H-quinolizinyl, decahydropyrido [1,2-a]azepinyl, decahydro-1H-pyrido [1,2-a]azocinyl, 1-azabicyclo[2.2.1]heptanyl, 1-azabicyclo[3.3.1]nonanyl, quinuclidinyl, and 1-azabicyclo[4.4.0]decanyl.

The term hydroxylalkyl” as used herein, means at least one hydroxyl group, as defined herein, is appended to the parent molecular moiety through an alkyl group, as defined herein. Illustrative examples of hydroxyalkyl include, but not limited to hydroxymethyl, 2-hydroxy-ethyl, 3-hydroxypropyl and 4-hydroxyheptyl.

The term “NR_{A}NR_{B}” as used herein, means two group, R_{A} and R_{B}, which are appended to the parent molecular moiety through a nitrogen atom. R_{A} and R_{B} are each independently hydrogen, alkyl, and alkylcarbonyl. Illustrative examples of NR_{A}R_{B} include, but are not limited to, amino, methylamino, acetylamino, and acetylmethylamino.

The term “(NR_{A}NR_{B})carbonyl” as used herein, means a R_{A}R_{B}, group, as defined herein, appended to the parent molecular moiety through a carbonyl group, as defined herein. Illustrative examples of (NR_{A}R_{B})carbonyl include, but are not limited to, aminocarbonyl, (methylamino)carbonyl, (dimethylamino)carbonyl, and (ethylmethylamino)carbonyl.

The term “NR_{C}NR_{D}” as used herein, means two group, R_{C} and R_{D}, which are appended to the parent molecular moiety through a nitrogen atom. R_{C} and R_{D} are each independently hydrogen, alkyl, and alkylcarbonyl. Illustrative examples of NR_{C}R_{D} include, but are not limited to, amino, methylamino, acetylamino, and acetylmethylamino.

The term “(NR_{C}NR_{D})carbonyl” as used herein, means a R_{C}R_{D}, group, as defined herein, appended to the parent molecular moiety through a carbonyl group, as defined herein. Illustrative examples of (NR_{C}R_{D})carbonyl include, but are not limited to, aminocarbonyl, (methylamino)carbonyl, (dimethylamino)carbonyl, and (ethylmethylamino)carbonyl.

As used herein, the term “mercaptyl” refers to a (alkyl)S— group.

As used herein, the term “moiety” refers to a specific segment or functional group of a molecule. Chemical moieties are often recognized chemical entities embedded in or appended to a molecule.

As used herein, the term “sulfinyl” refers to a —S(═O)—R, where R is selected from the group consisting of alkyl, cycloalkyl, aryl, heteroaryl (bonded through a ring carbon) and heterocycloalkyl (bonded through a ring carbon).

As used herein, the term “sulfonyl” refers to a —S(=O)₂—R, where R is selected from the group consisting of alkyl, cycloalkyl, aryl, heteroaryl (bonded through a ring carbon) and heterocycloalkyl (bonded through a ring carbon).

As used herein, the term “O carboxy” refers to a group of formula RC(═O)O—.

As used herein, the term “C carboxy” refers to a group of formula —C(═O)OR.

As used herein, the term “acetyl” refers to a group of formula —C(═O)CH₃.

As used herein, the term “trihalomethanesulfonyl” refers to a group of formula X₃CS(═O)₂— where X is a halogen.

As used herein, the term “isocyanato” refers to a group of formula —NCO.

As used herein, the term “thiocyanato” refers to a group of formula —CNS.

As used herein, the term “isothiocyanato” refers to a group of formula —NCS.

As used herein, the term “S sulfonamido” refers to a group of formula —S(=O)₂NR₂.

As used herein, the term “N sulfonamido” refers to a group of formula RS(=0)₂NH—.

As used herein, the term “trihalomethanesulfonamido” refers to a group of formula X₃CS(═O)₂NR—.

As used herein, the term “O carbamyl” refers to a group of formula —OC(═O)NR₂.

As used herein, the term “N carbamyl” refers to a group of formula ROC(═O)NH—.

As used herein, the term “O thiocarbamyl” refers to a group of formula —OC(═S)NR₂.

As used herein, the term “N thiocarbamyl” refers to a group of formula ROC(═S)NH—.

As used herein, the term “C amido” refers to a group of formula —C(═O)NR₂.

As used herein, the term “N amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

In some embodiments, the term “substituted” means that the referenced group is substituted with one or more additional group(s) individually and independently selected from alkyl, cycloalkyl, aryl, heteroaryl, heterocycloalkyl, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example, in some embodiments, an optional substituent is LₛRₛ, wherein each Lₛ is independently selected from a bond, —O—, —C(═O)—, —S—, —S(═O)—, —S(=O)₂—, —NH—, —NHC(O)—, —C(O)NH—, S(═O)₂NH—, —NHS(=O)₂, —OC(O)NH—, —NHC(O)O—, -(substituted or unsubstituted C₁-C₆ alkyl), or -(substituted or unsubstituted C₂-C₆ alkenyl); and each Rs is independently selected from H, (substituted or unsubstituted lower alkyl), (substituted or unsubstituted lower cycloalkyl), heteroaryl, or heteroalkyl.

The term “protecting group” refers to a removable group which modifies the reactivity of a functional group, for example, a hydroxyl, ketone or amine, against undesirable reaction during synthetic procedures and to be later removed. Examples of hydroxy-protecting groups include, but not limited to, methylthiomethyl, tert-dimethylsilyl, tert-butyldiphenylsilyl, ethers such as methoxymethyl, and esters including acetyl, benzoyl, and the like. Examples of ketone protecting groups include, but not limited to, ketals, oximes, O-substituted oximes for example O-benzyl oxime, O-phenylthiomethyl oxime, 1-isopropoxycyclohexyl oxime, and the like. Examples of amine protecting groups include, but are not limited to, tert-butoxycarbonyl (Boc) and carbobenzyloxy (Cbz).

In some embodiments, the term “optionally substituted” as defined herein, means the referenced group is substituted with zero, one or more substituents as defined herein.

The term “protected-hydroxy” refers to a hydroxy group protected with a hydroxy protecting group, as defined above.

In some embodiments, compounds described herein exist as stereoisomers, wherein asymmetric or chiral centers are present. Stereoisomers are designated (R) or (S) depending on the configuration of substituents around the chiral carbon atom. The term (R) and (S) used herein are configurations as defined in IUPAC 1974 Recommendations for Section E, Fundamental Stereochemistry, Pure Appl. Chem., (1976), 45:13-30, hereby incorporated by reference. The embodiments described herein specifically includes the various stereoisomers and mixtures thereof. Stereoisomers include enantiomers, diastereomers, and mixtures of enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic column.

The methods and formulations described herein include the use of N-oxides, crystalline forms (also known as polymorphs), or pharmaceutically acceptable salts of compounds described herein, as well as active metabolites of these compounds having the same type of activity. In some situations, compounds exist as tautomers. All tautomers are included within the scope of the compounds presented herein. In addition, in some embodiments, the compounds described herein exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof are chosen to provide, in some embodiments, stable moieties and compounds.

### Preparation of Compounds Described Herein

In some embodiments, the synthesis of agents that inhibit the activity of PARP are synthesized using standard synthetic techniques in combination with methods described herein. As a further guide the following synthetic methods are also utilized.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof.” Precursor functional groups are shown as electrophilic groups and nucleophilic groups. In some embodiments, the functional group on the organic substance is attached directly, or attached via any useful spacer or linker as defined below.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, e. g, oxygen or nitrogen.

### Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. In some embodiments, protective groups are removed by acid, base, and hydrogenolysis. Groups such as trityl, dimethoxytrityl, acetal and t-butyldimethylsilyl are acid labile and in some embodiments, is used to protect carboxy and hydroxy reactive moieties in the presence of amino groups protected with Cbz groups, which are removable by hydrogenolysis, and Fmoc groups, which are base labile. In some embodiments, carboxylic acid and hydroxy reactive moieties are blocked with base labile groups such as, without limitation, methyl, ethyl, and acetyl in the presence of amines blocked with acid labile groups such as t-butyl carbamate or with carbamates that are both acid and base stable but hydrolytically removable.

In some embodiments, carboxylic acid and hydroxy reactive moieties are also blocked with hydrolytically removable protective groups such as the benzyl group, while amine groups capable of hydrogen bonding with acids are blocked with base labile groups such as Fmoc. In some embodiments, carboxylic acid reactive moieties are protected by conversion to simple ester derivatives as exemplified herein, or they are blocked with oxidatively-removable protective groups such as 2,4-dimethoxybenzyl, while co-existing amino groups are blocked with fluoride labile silyl carbamates.

Allyl blocking groups are useful in then presence of acid- and base- protecting groups since the former are stable and in some embodiments, are subsequently removed by metal or pi-acid catalysts. For example, in some embodiments, an allyl-blocked carboxylic acid is deprotected with a Pd⁰-catalyzed reaction in the presence of acid labile t-butyl carbamate or base-labile acetate amine protecting groups. In some embodiments, another form of protecting group is a resin to which a compound or intermediate is attached. As long as the residue is attached to the resin, that functional group is blocked and cannot react. Once released from the resin, the functional group is available to react.

In some embodiments, typical blocking/protecting groups are selected from:

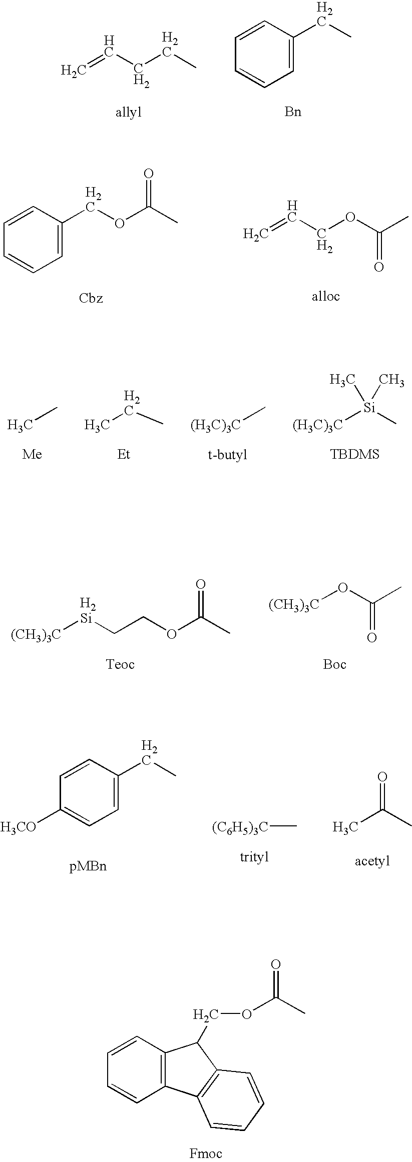

Other protecting groups are described in Greene and Wuts, Protective Groups in Organic Synthesis, 3rd Ed., John Wiley & Sons, New York, N.Y., 1999.

### Compounds of Formula (I)

In certain embodiments, compounds of Formula (I) are prepared in various ways, as outlined in Synthetic Scheme 1-3. In each scheme, the variables (e.g., R₁, R₂, R₃, R₄, R₅ and Y) correspond to the same definitions as those recited above. Compounds are synthesized using methodologies analogous to those described below by the use of appropriate alternative starting materials.

In some embodiments, compounds of Formula (I) are synthesized according to Synthetic Scheme 1 by condensation of the aldehyde 2 with phenylenediamines 1 in preferably polar solvents such as ethanol or dimethylformamide with addition of acids such as acetic acid at elevated temperature (ordinarily 80° C. to 120° C.), resulting in benzimidazole 3. It is beneficial for the reaction to add weak oxidizing agents such as copper (II) salts, iodine and the like, which are added as aqueous solution. (Bioorganic & Medicinal Letters, 14(10), 2433-2437; 2004).

When Z in the phenylenediamine 1 is NHR₅, the condensation reaction results directly in compounds of Formula (I) where R₄ is hydrogen. However, in other embodiments, if Z is O-Alkyl, this ester is reacted with ammonia or primary amine, where appropriate at elevated temperature and under elevated pressure, to give the compound of formula (I) where R₄ is hydrogen.

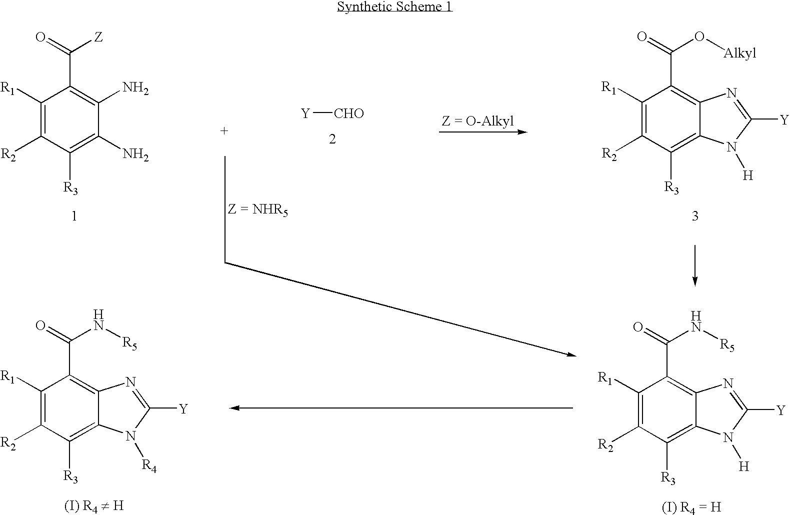

R₄ (other than hydrogen) is introduced into the benzimidazole (I) where R₄ is hydrogen by reaction with R₄-L where L is a leaving group under alkylating conditions.

Compounds of Formula (I) are also synthesized by replacing the aldehyde 2 with acid 4 (Synthetic Scheme 2) or nitrile 7 (Synthetic Scheme 3). These derivatives are prepared in analogy to the preparation of the substituted aldehyde 2.

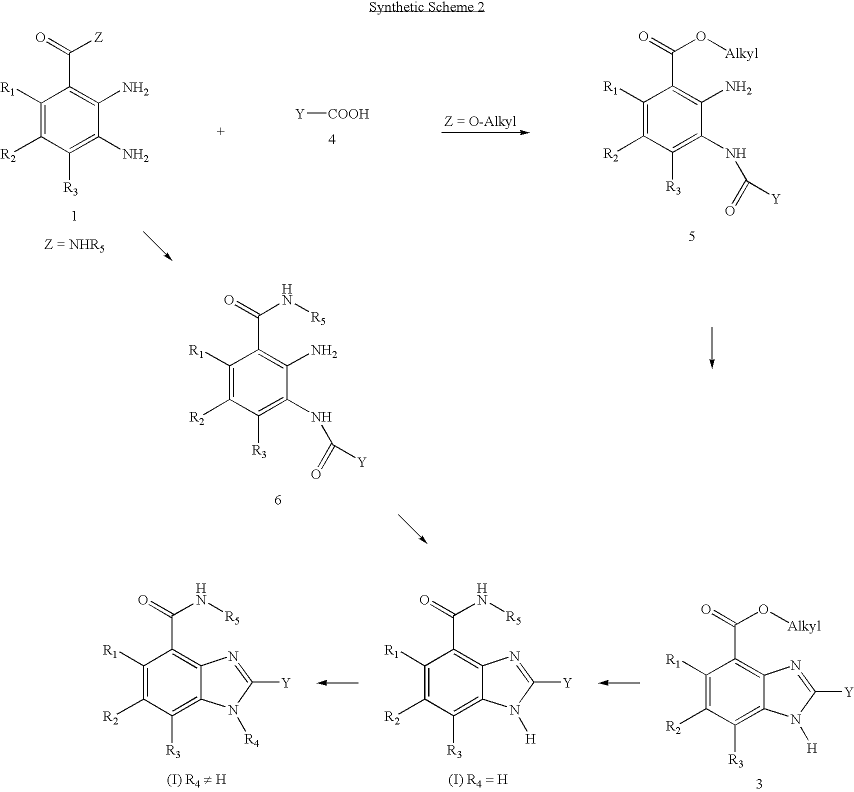

The condensation of the acid 4 with the phenylenediamines to generate 3 takes place in two stages. First, the acid 4 is reacted with the diamine 1 in a peptide-like coupling to give the amide 5. Conventional conditions are used for this kind of reaction. The ring closure to the benzimidazole then takes place at elevated temperature, for example 60-180° C., with or without solvents such as dimethylformamide, and with the addition of acids such as acetic acid or directly in acetic acid itself.

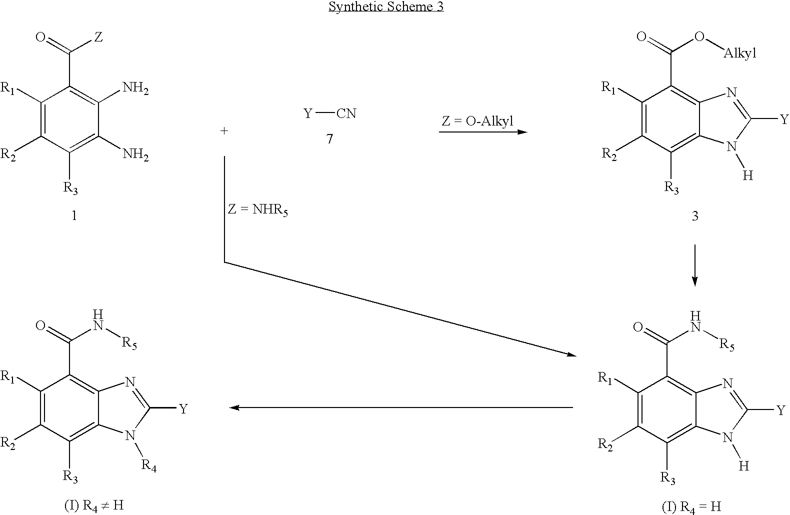

Reaction of the phenylenediamine 1 with the nitrile 7 likewise takes place under conventional conditions. It is moreover possible to use solvents such as dimethylformamide with the addition of acids or else use polyphosphoric acid at elevated temperature, such as 60-200° C. However, it is also possible to use the conventional methods for preparing amidines from benzonitriles.

### Certain Pharmaceutical Terminology

The term “acceptable” with respect to a formulation, composition or ingredient, as used herein, means having no persistent detrimental effect on the general health of the subject being treated.

As used herein, the term “selective binding compound” refers to a compound that selectively binds to any portion of one or more target proteins.

As used herein, the term “selectively binds” refers to the ability of a selective binding compound to bind to a target protein, such as, for example, PARP, with greater affinity than it binds to a non-target protein. In certain embodiments, specific binding refers to binding to a target with an affinity that is at least about 10, about 50, about 100, about 250, about 500, about 1000 or more times greater than the affinity for a non-target.

As used herein, the term “target protein” refers to a molecule or a portion of a protein capable of being bound by a selective binding compound. In certain embodiments, a target protein is the enzyme poly(ADP-ribose)polymerase (PARP).

As used herein, the terms “treating” or “treatment” encompass either or both responsive and prophylaxis measures, e.g., designed to inhibit, slow or delay the onset of a symptom of a disease or disorder, achieve a full or partial reduction of a symptom or disease state, and/or to alleviate, ameliorate, lessen, or cure a disease or disorder and/or its symptoms.

As used herein, amelioration of the symptoms of a particular disorder by administration of a particular compound or pharmaceutical composition refers to any lessening of severity, delay in onset, slowing of progression, or shortening of duration, whether permanent or temporary, lasting or transient that in some embodiments, is attributed to or associated with administration of the compound or composition.

As used herein, the term “modulator” refers to a compound that alters an activity of a molecule. For example, in some embodiments, a modulator causes an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “PARP” refers to the family of the enzyme poly(ADP-ribose)polymerase which includes approximately 18 proteins, particularly poly(ADP-ribose)polymerase-1 (PARP-1) and poly(ADP-ribose)polymerase-2 (PARP-2).

As used herein, the term “selective PARP modulator” refers to a compound that selectively modulates at least one activity associated with the enzyme poly(ADP-ribose)polymerase (PARP). In some embodiments, it selectively modulates the activity of PARP -1, PARP-2, both PARP-1 and PARP-2 or several members of the family of the enzyme poly(ADP-ribose)polymerase (PARP).

As used herein, the term “method of inhibiting PARP” refers to a method of inhibiting the activity of either one or more of the family of enzyme poly(ADP-ribose)polymerase (PARP). As used herein, the term “inhibition of PARP” refers to inhibition of the activity of either one or more of the family of enzyme poly(ADP-ribose)polymerase (PARP).

As used herein, the term “modulating the activity of the enzyme poly(ADP-ribose)polymerase” refers to a modulating the activity of either one or more of the family of enzyme poly(ADP-ribose)polymerase (PARP).

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, about 5, about 10, about 50, about 100, 1 about 50, about 200, about 250, about 300, about 350, about 400, about 450 or more than about 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “agonist” refers to a compound, the presence of which results in a biological activity of a protein that is the same as the biological activity resulting from the presence of a naturally occurring ligand for the protein, such as, for example, PARP.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” or “inhibitor” refers to a compound, the presence of which results in a decrease in the magnitude of a biological activity of a protein. In certain embodiments, the presence of an antagonist results in complete inhibition of a biological activity of a protein, such as, for example, the enzyme poly(ADP-ribose)polymerase (PARP).

As used herein, the IC₅₀ refers to an amount, concentration or dosage of a particular test compound that achieves a 50% inhibition of a maximal response, such as modulation of PARP, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “cancer”, as used herein refers to an abnormal growth of cells which tend to proliferate in an uncontrolled way and, in some cases, to metastasize (spread). The types of cancer include, but are not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias).

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. In some embodiments, diluents are also used to stabilize compounds because they provide a more stable environment. Salts dissolved in buffered solutions (which, in some embodiments, also provides pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. In some embodiments, the result is a reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. In some embodiments, an appropriate “effective” amount in any individual case is determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

In some embodiments, the term “enzymatically cleavable linker,” as used herein refers to unstable or degradable linkages which is degraded by one or more enzymes.

The term “inflammatory disorders” refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which in some embodiments, is partial or complete, temporary or permanent). Inflammation takes many forms and includes, but is not limited to, inflammation that is one or more of the following: acute, adhesive, atrophic, catarrhal., chronic, cirrhotic, diffuse, disseminated, exudative, fibrinous, fibrosing, focal., granulomatous, hyperplastic, hypertrophic, interstitial., metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (Chrohn's Disease, ulcerative colitis); skin (dermatitis); or multiple organs and tissues (systemic lupus erythematosus).

The term “PARP-mediated”, as used herein, refers to conditions or disorders that are ameliorated by the one or more of the family of enzyme poly(ADP-ribose)polymerase (PARP).

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in some embodiments, enzymes produce specific structural alterations to a compound. In some embodiments, metabolites of the compounds disclosed herein are identified either by administration of compounds to a host and analysis of tissue samples from the host, or by incubation of compounds with hepatic cells in vitro and analysis of the resulting compounds.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

By “pharmaceutically acceptable” or “therapeutically acceptable”, as used herein, refers a material, such as a carrier or diluent, which does not abrogate the biological activity or properties of the compound, and is relatively nontoxic, i.e., in some embodiments, the material is administered to an individual without causing undesirable biological effects or interacting in a deleterious manner with any of the components of the composition in which it is contained.

The term “pharmaceutically acceptable salt” or “therapeutically acceptable salt”, refers to a formulation of a compound that does not cause significant irritation to an organism to which it is administered and does not abrogate the biological activity and properties of the compound. In some embodiments, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some embodiments, pharmaceutically acceptable salts are also obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

A “prodrug” refers to an agent that is converted into the parent drug in vivo. Prodrugs are often useful because, in some situations, they are easier to administer than the parent drug. They are, for instance, bioavailable by oral administration whereas the parent is not. In some embodiments, the prodrug also has improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid or amino group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In some embodiments, the prodrug is designed to alter the metabolic stability or the transport characteristics of a drug, to mask side effects or toxicity, to improve the flavor of a drug or to alter other characteristics or properties of a drug.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Composition/Formulation

In some embodiments, pharmaceutical compositions are formulated in a conventional manner using one or more physiologically acceptable carriers comprising excipients and auxiliaries which facilitate processing of the active compounds into preparations which in some embodiments, are used pharmaceutically. Proper formulation is dependent upon the route of administration chosen. In some embodiments, any of the well-known techniques, carriers, and excipients are used as suitable.

Provided herein are pharmaceutical compositions that include a compound described herein and a pharmaceutically acceptable diluent(s), excipient(s), or carrier(s). In addition, in some embodiments, the compounds described herein are administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein provided herein are administered in a pharmaceutical composition to a mammal having a disease or condition to be treated. In one embodiment, the mammal is a human. In some embodiments, a therapeutically effective amount varies widely depending on the severity of the disease, the age and relative health of the subject, the potency of the compound used and other factors. In some embodiments, the compounds are used singly or in combination with one or more therapeutic agents as components of mixtures.

In some embodiments, for intravenous injections, compounds described herein are formulated in aqueous solutions, and in one embodiment, in physiologically compatible buffers such as Hank's solution, Ringer's solution, or physiological saline buffer. For transmucosal administration, penetrants appropriate to the barrier to be permeated are used in the formulation. In some embodiments, for other parenteral injections, appropriate formulations include aqueous or nonaqueous solutions, and in one embodiment, with physiologically compatible buffers or excipients.

In some embodiments, for oral administration, compounds described herein are formulated readily by combining the active compounds with pharmaceutically acceptable carriers or excipients. Such carriers enable the compounds described herein to be formulated as tablets, powders, pills, dragees, capsules, liquids, gels, syrups, elixirs, slurries, suspensions and the like, for oral ingestion by a patient to be treated.

In some embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In some embodiments, disintegrating agents are added, such as the cross-linked croscarmellose sodium, polyvinylpyrrolidone, agar, or alginic acid or a salt thereof such as sodium alginate.

Dragee cores are provided with suitable coatings. In some embodiments, concentrated sugar solutions are used, which optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs or pigments are added to the tablets or dragee coatings for identification or to characterize different combinations of active compound doses.

In some embodiments, pharmaceutical preparations which are used orally include push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches, and/or lubricants such as talc or magnesium stearate and, optionally, stabilizers. In some embodiments of soft capsules, the active compounds are dissolved or suspended in suitable liquids, such as fatty oils, liquid paraffin, or liquid polyethylene glycols. In addition, in some embodiments, stabilizers are added. All formulations for oral administration should be in dosages suitable for such administration.

For buccal or sublingual administration, in some embodiments, the compositions take the form of tablets, lozenges, or gels formulated in a conventional manner. In some embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the active compounds in water-soluble form. Additionally, in some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, the suspension also contains suitable stabilizers or agents which increase the solubility of the compounds to allow for the preparation of highly concentrated solutions. Alternatively, the active ingredient is in powder form for constitution with a suitable vehicle, e.g., sterile pyrogen-free water, before use.

In some embodiments, the compounds described herein are administered topically and in some embodiments, are formulated into a variety of topically administrable compositions, such as solutions, suspensions, lotions, gels, pastes, medicated sticks, balms, creams or ointments. In some embodiments, such pharmaceutical compounds contain solubilizers, stabilizers, tonicity enhancing agents, buffers and preservatives.

In some embodiments, formulations suitable for transdermal administration of compounds described herein employ transdermal delivery devices and transdermal delivery patches and in some embodiments, are lipophilic emulsions or buffered, aqueous solutions, dissolved and/or dispersed in a polymer or an adhesive. In some embodiments, such patches are constructed for continuous, pulsatile, or on demand delivery of pharmaceutical agents. Still further, in some embodiments, transdermal delivery of the compounds described herein are accomplished by means of iontophoretic patches and the like. Additionally, in some embodiments, transdermal patches provide controlled delivery of the compounds provided herein, such as, for example, compounds of Formula (I). The rate of absorption can be slowed by using rate-controlling membranes or by trapping the compound within a polymer matrix or gel. Conversely, absorption enhancers can be used to increase absorption. An absorption enhancer or carrier in some embodiments include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, in some embodiments, the compounds described herein are in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In some embodiments, in the case of a pressurized aerosol, the dosage unit is determined by providing a valve to deliver a metered amount. In some embodiments, capsules and cartridges of, such as, by way of example only, gelatin for use in an inhaler or insufflator are formulated containing a powder mix of the compound described herein and a suitable powder base such as lactose or starch.

In some embodiments, the compounds described herein are also formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, pharmaceutical compositions are formulated in conventional manner using one or more physiologically acceptable carriers comprising excipients and auxiliaries which facilitate processing of the active compounds into preparations which in some embodiments, is used pharmaceutically. Proper formulation is dependent upon the route of administration chosen. In some embodiments, any of the well-known techniques, carriers, and excipients are used as suitable and as understood in the art. In some embodiments, pharmaceutical compositions comprising a compound described herein are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and a compound described herein described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds exist as tautomers. All tautomers are included within the scope of the compounds presented herein. Additionally, in some embodiments, the compounds described herein exist in unsolvated as well as solvated forms with pharmaceutically acceptable solvents such as water, ethanol, and the like. The solvated forms of the compounds presented herein are also considered to be disclosed herein. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, in some embodiments, the pharmaceutical compositions also contain other therapeutically valuable substances.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In some embodiments, the compositions are in liquid solutions or suspensions, solid forms suitable for solution or suspension in a liquid prior to use, or as emulsions. In some embodiments, these compositions also contain minor amounts of nontoxic, auxiliary substances, such as wetting or emulsifying agents, pH buffering agents, and so forth.

In some embodiments, a composition comprising a compound described herein illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In some embodiments, useful aqueous suspensions also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. In some embodiments, useful compositions also comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, useful compositions also include solubilizing agents to aid in the solubility of a compound described herein. The term “solubilizing agent” generally includes agents that result in formation of a micellar solution or a true solution of the agent. In some embodiments, certain acceptable nonionic surfactants, for example polysorbate 80, are useful as solubilizing agents, as do ophthalmically acceptable glycols, polyglycols, e.g., polyethylene glycol 400, and glycol ethers.

In some embodiments, useful compositions also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, useful compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, other useful compositions also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. Alternatively, in some embodiments, multiple-dose reclosable containers are used, in which case it is typical to include a preservative in the composition.

Alternatively, other delivery systems for hydrophobic pharmaceutical compounds are employed. Liposomes and emulsions are well known examples of delivery vehicles or carriers for hydrophobic drugs. In some embodiments, certain organic solvents such as N-methylpyrrolidone also are employed, although usually at the cost of greater toxicity. Additionally, in some embodiments, the compounds are delivered using a sustained-release system, such as semi permeable matrices of solid hydrophobic polymers containing the therapeutic agent. Various sustained-release materials have been established and are well known by those skilled in the art. In some embodiments, sustained-release capsules, depending on their chemical nature, release the compounds for a few weeks up to over 100 days. Depending on the chemical nature and the biological stability of the therapeutic reagent, additional strategies, in some embodiments, for protein stabilization are employed.

In some embodiments, many of the formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) about 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

In some embodiments, the compounds described herein are used in the preparation of medicaments for the treatment of diseases or conditions that are mediated by the enzyme poly(ADP-ribose)polymerase (PARP) or in which inhibition of the enzyme poly(ADP-ribose)polymerase (PARP) ameliorates the disease or condition. In addition, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable pro drug, or pharmaceutically acceptable solvate thereof, in therapeutically effective amounts to said subject.

In some embodiments, the compositions containing the compound(s) described herein are administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered appropriate for the caregiver to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered appropriate for the caregiver to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, in some embodiments, upon the doctor's discretion the administration of the compounds are administered chronically, that is, for an extended period of time, including throughout the duration of the patient's life in order to ameliorate or otherwise control or limit the symptoms of the patient's disease or condition.

In the case wherein the patient's status does improve, in some embodiments, upon the doctor's discretion the administration of the compounds are given continuously; alternatively, the dose of drug being administered is temporarily reduced or temporarily suspended for a certain length of time (i.e., a “drug holiday”). The length of the drug holiday in some embodiments, varies between 2 days and 1 year, including by way of example only, about 2 days, about 3 days, about 4 days, about 5 days, about 6 days, about 7 days, about 10 days, about 12 days, about 15 days, about 20 days, about 28 days, about 35 days, about 50 days, about 70 days, about 100 days, about 120 days, about 150 days, about 180 days, about 200 days, about 250 days, about 280 days, about 300 days, about 320 days, about 350 days, or about 365 days. In some embodiments, the dose reduction during a drug holiday is from about 10%-about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, the dosage or the frequency of administration, or both, in some embodiments, is reduced, as a function of the symptoms, to a level at which the improved disease, disorder or condition is retained. In some embodiments, patients require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but in some embodiments, are determined according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in one embodiment about 1-about 1500 mg per day. In some embodiments, the desired dose is conveniently presented in a single dose or as divided doses administered simultaneously (or over a short period of time) or at appropriate intervals, for example as two, three, four or more sub-doses per day.

In some embodiments, the pharmaceutical composition described herein is in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, in some embodiments, multiple-dose reclosable containers are used, in which case it is typical to include a preservative in the composition. By way of example only, in some embodiments, formulations for parenteral injection are presented in unit dosage form, which include, but are not limited to ampoules, or in multi-dose containers, with an added preservative.

The daily dosages appropriate for the compounds described herein are from about 0.01 to about 75 mg/kg per body weight. In some embodiments, the daily dosage is from about 0. 1 to about 2.5 mg/kg per body weight. In some embodiments, an indicated daily dosage in the larger subject, including, but not limited to, humans, is in the range from about 0.5 mg to about 100 mg, conveniently administered in divided doses, including, but not limited to, up to four times a day or in extended release form. Suitable unit dosage forms for oral administration comprise from about 1 to about 50 mg active ingredient. The foregoing ranges are merely suggestive, as the number of variables in regard to an individual treatment regime is large, and considerable excursions from these recommended values are not uncommon. In some embodiments, such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies in some embodiments, is used in formulating a range of dosage for use in human. The dosage of such compounds lies preferably within a range of circulating concentrations that include the ED₅₀ with minimal toxicity. In some embodiments, the dosage varies within this range depending upon the dosage form employed and the route of administration utilized.

### Combination Treatments

In certain instances, it is appropriate to administer at least one compound described herein in combination with another therapeutic agent. By way of example only, if one of the side effects experienced by a patient upon receiving one of the compounds herein is inflammation, then in some embodiments, it is appropriate to administer an anti-inflammatory agent in combination with the initial therapeutic agent. Or, by way of example only, in some embodiments, the therapeutic effectiveness of one of the compounds described herein are enhanced by administration of an adjuvant (i.e., in some embodiments, by itself the adjuvant has minimal therapeutic benefit, but in combination with another therapeutic agent, the overall therapeutic benefit to the patient is enhanced). Or, in some embodiments, by way of example only, the benefit experienced by a patient increases by administering one of the compounds described herein with another therapeutic agent (which also includes a therapeutic regimen) that also has therapeutic benefit. In some embodiments, regardless of the disease, disorder or condition being treated, the overall benefit experienced by the patient is simply additive of the two therapeutic agents or the patient experiences a synergistic benefit.

In some embodiments, therapeutically-effective dosages vary when the drugs are used in treatment combinations. Methods for experimentally determining therapeutically-effective dosages of drugs and other agents for use in combination treatment regimens are described in the literature, e.g., the use of metronomic dosing, i.e., providing more frequent, lower doses in order to minimize toxic side effects. In some embodiments, a combination treatment regimen encompasses treatment regimens in which administration of a PARP inhibitor described herein is initiated prior to, during, or after treatment with a second agent described above, and continues until any time during treatment with the second agent or after termination of treatment with the second agent. It also includes treatments in which a PARP inhibitor described herein and the second agent being used in combination are administered simultaneously or at different times and/or at decreasing or increasing intervals during the treatment period. Combination treatment futher includes periodic treatments that start and stop at various times to assist with the clinical management of the patient. For example, in some embodiments, a PARP inhibitor described herein in the combination treatment is administered weekly at the onset of treatment, decreasing to biweekly, and decreasing further as appropriate.

Compositions and methods for combination therapy are provided herein. In accordance with one aspect, the pharmaceutical compositions disclosed herein are used to a PARP mediated disease or condition or a disease or condition that is ameliorated by inhibition of PARP. In accordance with another aspect, the pharmaceutical compositions disclosed herein are used to treat vascular disease; septic shock; ischaemic injury; reperfusion injury; neurotoxicity; haemorraghic shock; inflammatory diseases; multiple sclerosis; secondary effects of diabetes; and acute treatment of cytotoxicity following cardiovascular surgery. In another aspect, the pharmaceutical compositions disclosed herein are used in combination, either simultaneously or sequentially, with ionizing radiation or one or more chemotherapeutic agents.

In some embodiments, combination therapies described herein are used as part of a specific treatment regimen intended to provide a beneficial effect from the co-action of a PARP inhibitor described herein and a concurrent treatment. It is understood that the dosage regimen to treat, prevent, or ameliorate the condition(s) for which relief is sought, is, in some embodiments, modified in accordance with a variety of factors.

For combination therapies described herein, dosages of the co-administered compounds will of course vary depending on the type of co-drug employed, on the specific drug employed, on the disease or condition being treated and so forth. In some embodiments,, when co-administered with one or more biologically active agents, the compound provided herein is administered either simultaneously with the biologically active agent(s), or sequentially. If administered sequentially, the attending physician will decide on the appropriate sequence of administering protein in combination with the biologically active agent(s).

In some embodiments, the multiple therapeutic agents (one of which is one of the compounds described herein) is administered in any order or even simultaneously. If simultaneously, the multiple therapeutic agents in some embodiments, are provided in a single, unified form, or in multiple forms (by way of example only, either as a single pill or as two separate pills). In some embodiments, one of the therapeutic agents is given in multiple doses, or both is given as multiple doses. In some embodiments, if not simultaneous, the timing between the multiple doses varies from more than zero weeks to less than four weeks. In addition, the combination methods, compositions and formulations are not to be limited to the use of only two agents; the use of multiple therapeutic combinations are also envisioned.

In some embodiments, the compounds described herein are used in combination with procedures that provide additional or synergistic benefit to the patient. By way of example only, patients are expected to find therapeutic and/or prophylactic benefit in the methods described herein, wherein pharmaceutical composition of a compound dislcosed herein and /or combinations with other therapeutics are combined with genetic testing to determine whether that individual is a carrier of a mutant gene that is known to be correlated with certain diseases or conditions.

In other embodiments, the compounds described herein and combination therapies are administered before, during or after the occurrence of a disease or condition, and the timing of administering the composition containing a compound in some embodiments, varies. Thus, for example, in further embodiments, the compounds are used as a prophylactic and in yet further embodiments are administered continuously to subjects with a propensity to develop conditions or diseases in order to prevent the occurrence of the disease or condition. In other embodiments, the compounds and compositions are administered to a subject during or as soon as possible after the onset of the symptoms. In some embodiments, the administration of the compounds is initiated within the first 48 hours of the onset of the symptoms, within the first 6 hours of the onset of the symptoms, or within 3 hours of the onset of the symptoms. In some embodiments, the initial administration is via any route practical, such as, for example, an intravenous injection, a bolus injection, infusion over 5 minutes to about 5 hours, a pill, a capsule, transdermal patch, buccal delivery, and the like, or combination thereof. A compound is administered as soon as is practicable after the onset of a disease or condition is detected or suspected, and for a length of time necessary for the treatment of the disease, such as,, from about 1 month to about 3 months. In some embodiments, the length of treatment varies for each subject, and in some embodiments, the length is determined using the known criteria. For example, in some embodiments, the compound or a formulation containing the compound is administered for at least 2 weeks, for about 1 month to about 5 years, or for about 1 month to about 3 years.

### Other Combination Therapies

In another embodiment described herein, methods for treatment of PARP mediated conditions or diseases, such as proliferative disorders, including cancer, include administration to a patient compounds, pharmaceutical compositions, or medicaments described herein in combination with at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, and dronabinol.

### Kits/Articles of Manufacture

For use in the therapeutic applications described herein, kits and articles of manufacture are also described herein. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

The articles of manufacture provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

For example, in some embodiments, the container(s) comprise one or more compounds described herein, optionally in a composition or in combination with another agent as disclosed herein. The container(s) optionally have a sterile access port (for example, in some embodiments, the container is an intravenous solution bag or a vial having a stopper pierceable by a hypodermic injection needle). Such kits optionally comprising a compound with an identifying description or label or instructions relating to its use in the methods described herein.

In some embodiments, a kit will typically comprise one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. In some embodiments, a set of instructions will optionally be included.

In some embodiments, the label is on or associated with the container. In some embodiments, a label is on a container when letters, numbers or other characters forming the label are attached, molded or etched into the container itself; in some embodiments, a label is associated with a container when it is present within a receptacle or carrier that also holds the container, e.g., as a package insert. In some embodiments, a label is used to indicate that the contents are to be used for a specific therapeutic application. In some embodiments, the label also indicates directions for use of the contents, such as in the methods described herein.

In certain embodiments, the pharmaceutical compositions are presented in a pack or dispenser device which contains one or more unit dosage forms containing a compound provided herein. In some embodiments, the pack for example contains metal or plastic foil, such as a blister pack. In some embodiments, the pack or dispenser device optionally is accompanied by instructions for administration. In some embodiments, the pack or dispenser also is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, in some embodiments, is the labeling approved by the U.S. Food and Drug Administration for prescription drugs, or the approved product insert. In some embodiments, compositions containing a compound provided herein formulated in a compatible pharmaceutical carrier are also prepared, placed in an appropriate container, and labeled for treatment of an indicated condition.

### EXAMPLES

In order to assess the inhibitory action of the compounds, the following assay is used to determine IC₅₀ values (Dillon et al., JBS., 8(3), 347-352 (2003)).

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates™ (NEN, UK) and varying concentrations of said inhibitors added. All compounds were diluted in DMSO and gave final assay concentrations of between 10 and 0.0 1 μM, with the DMSO being at a final concentration of 1% per well. The total assay volume per well was 40 μL.

After 10 minutes incubation at 30° C., the reactions were initiated by the addition of a 10 μL reaction mixture, containing NAD (5 μM), ³H-NAD and 30 mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μL 30% acetic acid to each well. The plates were then shaken for 1 hour at room temperature.

The plates were transferred to a TopCount NXT™ (Packard, UK) for scintillation counting. Values recorded are counts per minute (cpm) following a 30 second counting of each well.

The % enzyme activity for each compound is then calculated using the following equation:

$$\% Inhibition = 100 - \{ 100 \times \frac{( cpm of unknown - mean negative cpm )}{( mean positive cpm - mean negative cpm )} \}$$

IC₅₀ values (the concentration at which 50% of the enzyme activity is inhibited) were calculated, which are determined over a range of different concentrations, normally from 10 μM down to 0.001 μM. Such IC₅₀ values are used as comparative values to identify increased compound potencies.

Most of the compounds tested had an IC₅₀ of less than 50 nM.

Chemosensitization assay determines the extent by which a PARP inhibitor enhances the tumor cell-killing effect of cytotoxic drugs expressed as PF50 (potentiation factor at GI50)]. 8000 LoVo cells were seeded into each well of a flat-bottomed 96-well microtiter plate in a volume of 50 μl and incubated in F12K containing 10% (v/v) FBS (medium) overnight at 37° C. Cells were added with 50 μl medium alone, medium containing 2 μM PARP inhibitor, medium containing increasing concentration of Temozolomide (0-2000 μM), and medium containing 2 μM PARP inhibitor and increasing concentration of Temozolomide (0-2000 μM). Final concentration range for Temozolomide was 0-1000 μM where applicable, final concentration of PARP inhibitor was 1 μM where applicable. Final concentration of DMSO is 1% in each well. Cells are allowed to grow for 5 days before cell survival was determined by CellTiter Glo staining (Promega, Madison, Wis., USA). Cell growth, determined after subtraction of time 0 values, was expressed as a percentage of the control well that contained medium with 1% DMSO. GI50 (concentration of drug that inhibited growth by 50%) values were calculated from the computer generated curves (GraphPad Software, Inc. San Diego Calif.). The potentiation factor [PF50 (potentiation factor at GI150)] was calculated as G150 of Temozolomide alone/G150 of Temozolomide+PARP inhibitor. Reference: Thomas H. D. et al. (2007). Preclinical selection of a novel poly(ADP-ribose) polymerase inhibitor for clinical trial. Molecular Cancer Therapy 6, 945-956.

A compound of Formula (I) was determined to have a PF50 of about 2.66.

### Xenograft Studies

### BRCA2-Deficient V-C8 or BRCA2-Complimented V-C8+B2 Cells

BRCA2-deficient V-C8 or BRCA2-complimented V-C8+B2 cells are implanted intramuscularly into the thigh of 40 CD-1 nude mice. Treatments are initiated when tumors are of measurable size (approximate leg diameter of 11 mm). Animals receive either a compound of Formula (I) (two doses of 25 or 50 mg/kg in saline) or saline (10 mg/ml) intraperitoneallly administered on days 1-5, and are monitored on a daily basis during treatment (tumor measurements, body weights and clinical evidence are recorded); and as required after the last treatment.

### ES-Cell-Derived Tumors

ES-cell-derived tumors (teratomas) are produced by subcutaneous injection of 2 x 106 ES cells into 6-8 week athymic BALB/c-nude (nu/nu) mice. 40 mice are injected with BRCA2-deficient ES cells or isogenic wild-type cells. Two days after cell injection, treatment with a compound of Formula (I) is initiated. For three consecutive days, two intraperitoneal doses of a compound of Formula (I) or vehicle is administered, 6 h apart, each at a dosage of 15 mg/kg per animal. This treatment is stopped for 5 days and then re-initiated for another three consecutive days. Growth of tumors is monitored from a minimum volume of 0.2 cm^{3.}

The in vitro assays disclosed herein, along with other known in vitro assays (Farmer et al., Nature 2005; 434:913-7: clonogenic survival assay finding that a BRCA2-deficient cell line V-C8, compared with the BRCA2 wild type control exhibited sensitivity to AG14361, a PARP-1 inhibitor, (Ki=5 nm) and NU1025, a moderately potent PARP-1 inhibitor (Ki=50 nM); & Mcabe et al, Cancer Biology & Therapy 2005; 4:9, 934-36; clonogenic survival assay using CAPAN-1 cells maintained in DMEM supplemented with FCS (20% v/v), glutamine and antibiotics showing sensitivity to PARP inhibition using KU0058684) demonstrates the activity of PARP-inhibitors in a static test situation. Additionally, animal models have been used to analyze the relationship between in vitro tests and parameters of in vivo efficacy. By way of example only, Farmer et al, has shown in vivo efficacy in blocking the growth of BRCA2-deficient tumors using KU0058684, a PARP-1 inhibitor. Nature 2005; 434:913-7. This indicates that PARP-1 inhibition is a viable cancer treatment for BRCA1/2 mutation carriers. Furthermore, KU0059436, a PARP-1 inhibitor is currently in Phase I clinical trials for patients with advanced solid tumors. Given this information, compounds of Formula (I) which have shown in vitro inhibitory action are likely to show analogous in vivo (mouse and human) efficacy.

### Phase II Clinical Trial of the Safety and Efficacy of Compounds of Formula (I)

The purpose of this phase II trial is to study the side effects and best dose of a compound of Formula (I) and to determine how well it works in treating patients with locally advanced or metastatic breast cancer or advanced ovarian cancer

### Objectives:

Primary:
A. To determine the response rate to a compound of Formula (I) in patients with locally advanced or metastatic breast or advanced ovarian cancer shown to express the BRCA 1 or 2 mutations
B. To evaluate the toxicity of a compound of Formula (I) in these patients

Secondary:
A. To evaluate the time to progression and overall survival in patients treated with a compound of Formula (I)
B. To study pharmacokinetics of a compound of Formula (I) in these patients
C. To evaluate the Poly(ADP-ribose) polymerase (PARP) activity in peripheral blood lymphocytes from BRCA 1 and 2 heterozygotic patients

Tertiary:
A. To evaluate PARP expression using quantitative western blotting immuno-assays
B. To investigate pharmacogenomics, including CYP2D6 and CYP3A5, drug transport proteins, as well as polymorphisms in the genes coding for the PARP enzymes themselves
C. To analyze tumor biopsy samples (when possible) for BRCA mutation status, PARP activity, and PARP expression
D. To analyze paraffin sections from original diagnostic biopsies/operative procedures (when available) for DNA repair enzyme status using immunohistochemical techniques
E. To analyze cells obtained from ascitic or pleural fluid (where available) for primary cell culture for DNA double strand break repair pathway function
   Patients: Eligible subjects will be men and women 18 years and older

### Criteria:

Disease Characteristics:
Histologically confirmed locally advanced or metastatic breast cancer or advanced ovarian cancer
Must meet 1 of the following criteria:
   Proven a carrier of a known mutation of BRCA 1 or BRCA 2
   Considered highly likely a carrier of BRCA 1 or 2 mutation (score of ≧20 per Manchester criteria)
No more than 3 prior chemotherapy regimens for patients with breast or ovarian cancer
   More than 2 months since prior carboplatin- or cisplatin-containing chemotherapy for ovarian cancer
Measurable disease, as defined by RECIST criteria and measured by x-ray, CT scan, or MRI
   Patients with bone disease must have other measurable disease for evaluation
   Previously irradiated lesions cannot be used for measurable disease
No known brain metastases
Hormone receptor status not specified

Patient Characteristics:
WHO performance status 0-1
Life expectancy ≧12 weeks
Menopausal status not specified
Hemoglobin ≧9.0 g/dL
Absolute neutrophils ≧1,500/mm³
Platelets≧100,000/mm³
Serum bilirubin ≦1.5 times upper limit of normal (ULN)
ALT or AST ≦2.5 times ULN (≦5 times ULN if due to tumor)
Glomerular filtration rate (GFR) ≧50 mL/min
Not pregnant or nursing
Negative pregnancy test
Fertile patients must use two highly effective forms of contraception (i.e., oral, injected, or implanted hormonal contraception, intrauterine device, barrier method of condom plus spermicide, or are surgically sterile) 4 weeks prior to (females), during, and for 6 months after (males and females) completion of study therapy
Able to cooperate with treatment and follow-up
No non-malignant systemic disease, including active uncontrolled infection
No other concurrent malignancy, except adequately treated cone-biopsied carcinoma in situ of the uterine cervix, basal cell or squamous cell carcinoma of the skin, or breast and ovarian carcinoma
   Cancer survivors who have undergone potentially curative therapy for a prior malignancy, have no evidence of that disease for 5 years, and are deemed at low risk for recurrence are eligible
No active or unstable cardiac disease or history of myocardial infarction within the past 6 months
   Patients with cardiovascular signs or symptoms should have a MUGA scan or echocardiogram, and those with a left ventricular ejection fraction (LVEF) below the institutional limit of normal should be excluded
No other condition which, in the investigator's opinion, would not make the patient a good candidate for this study

### Prior Concurrent Therapy:

At least 4 weeks since prior radiotherapy (except for palliative reasons), endocrine therapy, immunotherapy or chemotherapy (6 weeks for nitrosoureas and mitomycin C)

At least 4 weeks since prior major thoracic and/or abdominal surgery and recovered

Concurrent radiotherapy for the control of bone pain or skin lesions allowed, but not within 5 days of the last dose of study drug

Concurrent bisphosphonates allowed provided the dose is stable and treatment was started at least 2 weeks prior to recruitment

No unresolved toxicities (CTCAE≧grade 1) from prior treatments (except for alopecia)

No concurrent anticancer therapy or investigational drugs

No concurrent tetracycline antibiotic therapy for prolonged periods (short courses [5-7 days] for treatment of infection are allowed)

Study Design: This is a dose-escalation study followed by an open label multicenter study. Patients will be stratified according to tumor type (breast vs ovarian) and mutation status (BRCA 1 vs BRCA 2). Patients will receive a compound of Formula (I) (at one of several possible dosages) over 30 minutes once daily on days 1-5. Treatment repeats every 21 days for 12 courses in the absence of disease progression or unacceptable toxicity. Patients who achieve stable or responding disease may receive additional courses of treatment at the discretion of the chief investigator or Drug Development Office (DDO). Patients will undergo blood sample collection periodically for pharmacokinetic and pharmacodynamic studies. Samples will be analyzed for tumor marker (CA 125 or CA 15.3) measurements, plasma levels of a compound of Formula (I) via liquid chromatography/mass spectrometry/mass spectrometry, PARP activity, and PARP protein expression via western blotting immunoassays. Paraffin embedded sections from original diagnostic biopsy are also collected and analyzed for PARP protein expression via immunohistochemical technique. Pleural and ascitic fluid may be collected and analyzed for DNA DS break repair proficiency via immunohistochemical technique. Some patients will also undergo biopsy of tumors and samples will be analyzed for BRCA 2 mutation as well as PARP activity via validated PARP immunoblotting assay. After completion of the study treatment, patients will be followed for 28 days.

Primary Outcome Measures:
Assessment of antitumor activity according to RECIST using tumor size measured clinically or radiologically with CT scan, MRI, plain x-ray, or other imaging techniques
Safety profile

Secondary Outcome Measures:
Time to progression and overall survival
Plasma levels by Liquid Chromatography/Mass Spectrometry/Mass Spectrometry
Poly(ADP-ribose) polymerase (PARP) activity measured ex vivo using validated assays
PARP expression using quantitative Western blotting immuno-assays
Pharmacogenomics including CYP2D6 and CYP3A5, drug transport proteins, as well as polymorphisms in the genes coding for the PARP enzymes themselves
BRCA mutation status, PARP activity, and PARP expression in tumor biopsy samples (when possible)
DNA repair enzyme status using immunohistochemical techniques in paraffin sections from original diagnostic biopsies/operative procedures (where available)
DNA double strand break repair pathway function in cells obtained from ascitic or pleural fluid (where available) for primary cell culture
   Phase I Clinical Trial of the Combination of a Compound of Formula (I) with temozolomide (TMZ)

The purpose of this Phase I clinical trial is to assess the safety, tolerability and pharmacokinetic profile of a compound of Formula (I) in combination with temozolomide in subjects with metastatic melanoma

Patients: Eligible subjects will be men and women 18 years and older

### Criteria:

Inclusion Criteria:
Have histologically confirmed malignancy, either metastatic or unresectable and standard curative measures or other therapy that may provide clinical benefit do not exist or are no longer effective
Have evaluable disease
ECOG less than or equal to 2
Adequate hematologic, renal and hepatic function
All adverse events from prior treatment are resolved or stable
Voluntarily signed informed consent

Exclusion Criteria:
Known CNS metastases or CNS primary cancer
Previous history of or current seizure disorder
Have received any anti-cancer therapy including chemotherapy, immunotherapy, radiotherapy, hormonal, biologic or investigational therapy within 4 weeks
Not recovered to Grade 1 clinical significant adverse effects/toxicities of the previous therapy
Lactating or pregnant female
Subject is receiving combination anti-retroviral therapy for HIV
Prior therapy with regimens containing temozolomide (TMZ) are excluded
   Study Design: This is a non-randomized, open label, safety study.

Part I of the study requires that patients receive a compound of Formula (I) and temozolomide daily (x5) every 28 days. The temozolomide dose is half of standard (100 mg/m²/day po) and the compound of Formula (I) (given as a 30 min intravenous infusion) is escalated until the PARP-inhibitory dose (PID) as determined by PARP activity in peripheral blood lymphocytes (PBLs) is defined.

Part II of the study requires that the dose of a compound of Formula (I) is fixed at PID and temozolomide is escalated to the maximum tolerated dose or 200 mg/m²/day in metastatic melanoma pts. Endpoints will include safety, efficacy, PK, and tumor PARP activity.

The following Examples are intended as an illustration of the various embodiments as defined in the appended claims. In some embodiments, the compounds are prepared by a variety of synthetic routes. All publications, patents, and patent applications cited herein are hereby incorporated by reference for all purposes.

### Example 1

### Example 1a

### Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example 1b

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example 1c

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 1d

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 1e

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 1f

### Topical Gel Composition

To prepare a pharmaceutical topical gel composition, 100 mg of a compound described herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 1g

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example 2

### 2-((1R,4S)-2-azabicyclo[2.2.1]heptan-1-yl)-1H-benzo[d]imidazole-4-carboxamide-4-carboxamide

### Example 2A

### (1R,4S)-2-(benzyloxycarbonyl)-2-azabicyclo[2.2.1]heptanes-1-carboxylic acid

(1R,4S)-2-azabicyclo[2.2.1]heptane-1-carboxylic acid (11.0 g, 78 mmol) [described in Tetrahedron: Asymmetry, 17(2), 252-258; 2006], is added to a solution of methylene chloride (250 mL) and triethylamine (14 mL, 100 mmol) at ice-bath. Benzylchloroformate (13.4 g, 78.5 mmol) is added in dropwise. The reaction mixture is then stirred for 1 h and the ice-bath is removed. After stirring the reaction mixture for an additional 5 h, the mixture is washed with brine (100 mL). The organic layer is then separated and further washed with water. The organic layer is collected and dried over magnesium sulphate. Removal of the solvent under reduced pressure yields (1R,4S)-2-(benzyloxycarbonyl)-2-azabicyclo[2.2.1]heptanes-1-carboxylic acid.

### Example 2B

### (1R,4S)-benzyl 1-(2-amino-3-carbamoylphenylcarbamoyl)-2-arazabicyclo[2.2.1]heptanes-2-carboxylate

To a solution of (1R,4S)-2-(benzyloxycarbonyl)-2-azabicyclo[2.2.1]heptanes-1-carboxyl g, 52 mmol) in a mixture of pyridine (60 mL) and DMF (60 mL) is treated with 1,1′-carbonyldiimidazole (9.27 g, 57.2 mmol) at 45° C. for 2 h. 2,3-Diamino-benzamide dihydrochloride (11.66 g, 52 mmol) [described in US patent publication (U.S. Pat. No. 6,737,421 B1)], is added to the reaction mixture and the mixture is stirred at ambient temperature overnight. After concentration under vacuum, the residue is dissolved in methylene chloride and is washed with diluted sodium bicarbonate aqueous solution. The organic layer is separated and dried over magnesium sulphate and filtered. Concentration of the solution under vacuum gives a residue of (1R,4S)-benzyl 1-(2-amino-3-carbamoylphenylcarbamoyl)-2-arazabicyclo[2.2.1]heptanes-2-carboxylate.

### Example 2C

### (1R,4S)-benzyl 1-(4-carbamoyl-1H-benzo[d]imidazol-2-yl)-2-azabicyclo[2.2.1]heptane-2-carboxylate

A suspension of (1R,4S)-benzyl 1-(2-amino-3-carbamoylphenylcarbamoyl)-2-arazabicyclo[2.2.1]heptanes-2-carboxylate (17.8 g, 43.6 mmol) in acetic acid (180 mL) is heated under reflux for 3 h. After cooling, the solution is concentrated and the residue is partitioned between ethyl acetate and sodium bicarbonate aqueous solution. The organic phase is washed with water and concentrated. The residue is purified by flash column chromatography in silica gel yielding (1R,4S)-benzyl 1-(4-carbamoyl-1H-benzo[d]imidazol-2-yl)-2-azabicyclo[2.2.1]heptane-2-carboxylate.

### Example 2D

### 2-((1R,4S)-2-azabicyclo[2.2.1]heptan-1-yl)-1H-benzo[d]imidazole-4-carboxamide

A solution of (1R,4S)-benzyl 1-(4-carbamoyl-1H-benzo[d]imidazol-2-yl)-2-azabicyclo[2.2.1]heptane-2-carboxylate (15.6 g, 40 mmol) in methanol (250 mL) is treated with 10% Pd (2.8 g) under 60 psi of hydrogen for overnight. Solid material is filtered off and the filtrate is concentrated giving 2-((1R,4S)-2-azabicyclo[2.2.1]heptan-1-yl)-1H-benzo[d]imidazole-4-carboxamide.

### Example 3

### 2-((1S,4R)-2-azabicyclo[2.2.1]heptan-1-yl)-1H-benzo[d]imidazole-4-carboxamide

Following the experimental procedure as described in Examples 2A to 2D, and using (1S,4R)-2-azabicyclo[2.2.1]heptane-1-carboxylic acid [described in Tetrahedron: Asymmetry, 17(2), 252-258; 200 instead of (1R,4S)-2-azabicyclo[2.2.1]heptane-1-carboxylic acid, the title compound 2-((1S,4R)-2-azabicyclo[2.2.1]heptan-1-yl)-1H-benzo[d]imidazole-4-caroxamide is made.

### Example 4

### 2-(2-azabicyclo[2.2.1]heptan-1-yl)-1H-benzo[d]imidazole-4-carboxamide

Following the experimental procedure as described in Examples 2A to 2D, and using 2-azabicyclo[2.2.1]heptane-1-carboxylic acid [described in Tetrahedron: Asymmetry, 17(2), 252-258; 2006], instead of (1R,4S)-2-azabicyclo[2.2.1]heptane-1-carboxylic acid, the title compound 2-(2-azabicyclo[2.2.1]heptan-1-yl)-1H-benzo[d]imidazole-4-caroxamide is made.

### Example 5

### 2-(7-azabicyclo[2.2.1]heptan-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid, with 7-azabicyclo[2.2.1]heptane-1-carboxylic in Tetrahedron: Asymmetry, 17(2), 252-258; 2006 and Tetrahedron, 57(3) 545-548; 2001], the title compound 2-(7-azabicyclo[2.2.1]heptan-1-yl)-1H-benzo[d]imidazole-4-caroxamide is made.

### Example 6

### 2-(2-methyl-7-azabicyclo[2.2.1]heptan-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid, with 2-methyl-7-azabicyclo[2.2.1]heptane-1-carboxylic acid [described in Tetrahedron: Asymmetry, 16(18), 3115-3123; 2005], the title compound 2-(2-methyl-7-azabicyclo[2.2.1]heptan-1-yl)-1H-benzo[d]imidazole-4-caroxamide is made.

### Example 7

### 2-(2-azabicyclo[2.1.1]hexan-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 2-azabicyclo[2.1.1]hexane-1-carboxylic acid Journal Organic Chemistry, 67, 6509-6513; 2002], the title compound 2-(2-azabicyclo[2.1.1]hexan-1-yl)-1H-benzo[d]imidazole -4-carboxamide is made.

### Example 8

### 2-(6-azabicyclo[3.2.1]octan-5-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 6-azabicyclo[3.2.1]octane-1-carboxylic acid Tetrahedron: Asymmetry, 17(2), 252-258; 2006], the title compound 2-(6-azabicyclo[3.2.1]octan-5-yl)-1H-benzo[d]imidazole -4-carboxamide is made.

### Example 9

### 2-((1S,5R)-6-azabicyclo[3.2.1]octan-5-yl)-1H-benzo[d]imidazole-4-carboxamide

Following the experimental procedure as described in Example 8 and substituting 6-azabicyclo[3.2.1]octane-1-carboxylic acid with (1S,5R)-6-azabicyclo[3.2. 1]octane-1-carboxylic acid [described in Tetrahedron: Asymmetry, 17(2), 252-258; 2006], the title compound 2-((1S,5R)-6-azabicyclo[3.2.1]octan-5-yl)-1H-benzo[d]imidazole-4-carboxamide is made.

### Example 10

### 2-((1R1,5S)-6-azabicyclo[3.2.1]octan-5-yl)-1H-benzo[d]imidazole-4-carboxamide

Following the experimental procedure as described in Example 8 and substituting 6-azabicyclo[3.2.1]octane-1-carboxylic acid with (1R,5S)-6-azabicyclo[3.2.1]octane-1-carboxylic acid [described in Tetrahedron: Asymmetry, 17(2), 252-258; 2006], the title compound 2-((1R,5S)-6-azabicyclo[3.2. 1]octan-5-yl)-1H-benzo[d]imidazole -4-carboxamide is made.

### Example 11

### 2-(2-benzyl-2-azabicyclo[2.2.2]octan-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 2-benzyl-2-azabicyclo[2.2.2]octane-4-carboxamide [described in Tetrahedron, 62(42), 10000-10004; 2006], the title compound 2-(2-benzyl-2-azabicyclo[2.2.2]octan-1-yl)-1H-benzo[d]imidazole-4-carboxamide is made.

### Example 12

### 2-(2-azabicyclo[2.2.2]octan-1-yl)-1H-benzo[d]imidazole-4-carboxamide

Following the experimental procedure as described in Example 11 and substituting 2-benzyl-2-azabicyclo[2.2.2]octane-4-carboxylic acid with 2-azabicyclo[2.2.2]octane-4-carboxylic acid which is synthesized by hydrogenolysis of 2-benzyl-2-azabicyclo[2.2.2]octane-4-carboxylic acid [described in Tetrahedron, 62(42), 10000-10004; 2006] to remove the benzyl group, the title compound 2-(2-azabicyclo[2.2.2]octan-1-yl)-1H-benzo[d]imidazole-4-carboxamide is made.

### Example 13

### 2-(4-azaspiro[2.4]heptan-5-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 4-azaspiro[2.4]heptane-5-carboxylic acid [described in Tetrahedron Letters, 30(4), 399-402; 1989], the title compound 2-(4-azaspiro[2.4]heptan-5-yl)-1H-benzo[d]imidazole-4-carboxamide is made.

### Example 14

### 2-((1R,4S)-2-methyl-2-azabicyclo[2.2.1]heptan-1-yl)-1H-benzo[d]imidazole-4-carboxamide

A solution of 2-((1R,4S)-2-azabicyclo[2.2.1]heptan-1-yl)-1H-benzo[d]imidazole-4-carboxamide (310 mg, 1.30 mmol0 in methanol 920 mL) is treated with formaldehyde (37 wt % in water, 250 μL, 3.37 mmol) at room temperature for overnight. Sodium cyanoborohydride (212 mg, 3.37 mmol) is added and the solution is stirred at room temperature for 3 h. After concentration under reduced pressure, the residue is dissolved in a mixture of trifluoroacetic acid and water and is purified by HPLC giving the title compound 2-((1R,4S)-2-methyl-2-azabicyclo[2.2.1]heptan-1-yl)-1H-benzo[d]imidazole-4-carboxamide.

### Example 15

### 2-((1R,4S)-2-ethyl-2-azabicyclo[2.2.1]heptan-1-yl)-1H-benzo[d]imidazole-4-carboxamide

The title compound, 2-((1R,4S)-2-ethyl-2-azabicyclo[2.2.1]heptan-1-yl)-1H-benzo[d]imidazol-4-carboxamide is prepared according to the procedure for Example 14, substituting acetaldehyde for formaldehyde.

### Example 16

### 2-((1R,4S)-2-cyclopropyl-2-azabicyclo[2.2.1]heptan-1-yl)-1H-benzo[d]imidazole-4-carboxamide

The title compound, 2-((1R,4S)-2-cyclopropyl-2-azabicyclo[2.2.1]heptan-1-yl)-1H-benzo[d]imidazole-4-carboxamide is prepared according to the procedure for Example 14, substituting cyclopropanecarbaldehyde for formaldehyde.

### Example 17

### 2-((1R,4S)-2-isoproppyl-2-azabicyclo[2.2.1]heptan-1-yl)-1H-benzo[d]imidazole-4-carboxamide

The title compound, 2-((1R,4S)-2-isopropyl-2-azabicyclo[2.2.1]heptan-1-yl)-1H-benzo[d]imidazole-4-carboxamide is prepared according to the procedure for Example 14, substituting isobutyraldehyde for formaldehyde.

### Example 18

### 2-((1R,4S)-2-cyclohexyl-2-azabicyclo[2.2.1]heptan-1-yl)-1H-benzo[d]imidazole-4-carboxamide

The title compound, 2-((1R,4S)-2-cyclohexyl-2-azabicyclo[2.2.1]heptan-1-yl)-1H-benzo[d]imidazole-4-carboxamide is prepared according to the procedure for Example 14, substituting cyclohexanecarbaldehyde for formaldehyde.

### Example 19

### 2-((1S,4R)-2-methyl-2-azabicyclo[2.2.1]heptan-1-yl)-1H-benzo[d]imidazole-4-carboxamide

A solution of 2-((1S,4R)-2-azabicyclo[2.2.1]heptan-1-yl)-1H-benzo[d]imidazole-4-carboxamide (280 mg, 1.17 mmol) in methanol (20 mL) is treated with formaldehyde (37 wt % in water, 225 pL, 3.03 mmol) at room temperature for overnight. Sodium cyanoborohydride (190 mg, 3.03 mmol) is added and the solution is stirred at room temperature for 3 h. After concentration under reduced pressure, the residue is dissolved in a mixture of trifluoroacetic acid and water and is purified by HPLC giving the title compound 2-((1S,4R)-2-methyl-2-azabicyclo[2.2.1]heptan-1-yl)-1H-benzo[d]imidazole-4-carboxamide.

### Example 20

### 2-((1S,4R)-2-ethyl-2-azabicyclo[2.2.1]heptan-1-yl)-1H-benzo[d]imidazole-4-carboxamide

The title compound, 2-((1S,4R)-2-ethyl-2-azabicyclo[2.2.1]heptan-1-yl)-1H-benzo[d]imidazole-4-carboxamide is prepared according to the procedure for Example 19, substituting acetaldehyde for formaldehyde.

### Example 21

### 2-((1S,4R)-2-cyclopropyl-2-azabicyclo[2.2.1]heptan-1-yl)-1H-benzo[d]imidazole-4-carboxamide

The title compound, 2-((1S,4R)-2-cyclopropyl-2-azabicyclo[2.2.1]heptan-1-yl)-1H-benzo[d]imidazole-4-carboxamide is prepared according to the procedure for Example 19, substituting cyclopropanecarbaldehyde for formaldehyde.

### Example 22

### 2-((1R,4S)-2-propyl-2-azabicyclo[2.2.1]heptan-1-yl)-1H-benzo[d]imidazole-4-carboxamide

The title compound, 2-((1R,4S)-2-propyl-2-azabicyclo[2.2.1]heptan-1-yl)-1H-benzo[d]imidazole-4-carboxamide carboxamide is prepared according to the procedure for Example 19, substituting butyraldehyde for formaldehyde.

### Example 23

### 2-((1R,4S)-2-cyclobutyl-2-azabicyclo[2.2.1]heptan-1-yl)-1H-benzo[d]imidazole-4-carboxaxamide

The title compound, 2-((1R,4S)-2-cyclobutyl-2-azabicyclo[2.2.1]heptan-1-yl)-1H-benzo[d]imidazole-4-carboxamide is prepared according to the procedure for Example 19, substituting cyclobutanecarbaldehyde for formaldehyde.

### Example 24

### 2-(2-methyl-2-azabicyclo[2.2.1]heptan-1-yl)-1H-benzo[d]imidazole-4-carboxamide

A solution of 2-(2-azabicyclo[2.2.1]heptan-1-yl)-1H-benzo[d]imidazole-4-carboxamide (336 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-(2-methyl-2-azabicyclo[2.2.1]heptan-1-yl)-1H-benzo[d]imidazole-4-carboxamide.

### Example 25

### 2-(2-ethyl-2-azabicyclo[2.2.1]heptan-1-yl)-1H-benzo[d]imidazole-4-carboxamide

The title compound, 2-(2-ethyl-2-azabicyclo[2.2.1]heptan-1-yl)-1H-benzo[d]imidazole-4-carboxamide is prepared according to the procedure for Example 24, substituting acetaldehyde for formaldehyde.

### Example 26

### 2-(2-cyclopropyl-2-azabicyclo[2.2.1]heptan-1-yl)-1H-benzo[d]imidazole-4-carboxamide

The title compound, 2-(2-cyclopropyl-2-azabicyclo[2.2.1]heptan-1-yl)-1H-benzo[d]imidazole-4-carboxamide is prepared according to the procedure for Example 24, substituting cyclopropanecarbaldehyde for formaldehyde.

### Example 27

### 2-(2-isopropyl-2-azabicyclo[2.2.1]heptan-1-yl)-1H-benzo[d]imidazole-4-carboxamide

The title compound, 2-(2-isopropyl-2-azabicyclo[2.2.1]heptan-1-yl)-1H-benzo[d]imidazole-4-carboxamide is prepared according to the procedure for Example 24, substituting isobutyraldehyde for formaldehyde.

### Example 28

### 2-(2-cyclopentyl-2-azabicyclo[2.2.1]heptan-1-yl)-1H-benzo[d]imidazole-4-carboxamide

The title compound, 2-(2-cyclopentyl-2-azabicyclo[2.2.1]heptan-1-yl)-1H-benzo[d]imidazole-4-carboxamide is prepared according to the procedure for Example 24, substituting cyclopentanecarbaldehyde for formaldehyde.

### Example 29

### 2-(2-methyl-2-azabicyclo[2.1.1]hexan-1-yl)-1H-benzo[d]imidazole-4-carboxamide

A solution of 2-(2-azabicyclo[2.1.1]hexan-1-yl)-1H-benzo[d]imidazole-4-carboxamide (340 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-(2-methyl-2-azabicyclo[2.1.1]hexan-1-yl)-1H-benzo[d]imidazole-4-carboxamide.

### Example 30

### 2-(2-ethyl-2-azabicyclo[2.1.1]hexan-1-yl)-1H-benzo[d]imidazole-4-carboxamide

The title compound, 2-(2-ethyl-2-azabicyclo[2.1.1]hexan-1-yl)-1H-benzo[d]imidazole-4-carboxamide is prepared according to the procedure for Example 29, substituting acetaldehyde for formaldehyde.

### Example 31

### 2-(2-cyclopropyl-2-azabicyclo[2.1.1]hexan-1-yl)-1H-benzo[d]imidazole-4-carboxamide

The title compound, 2-(2-cyclopropyl-2-azabicyclo[2.1.1]hexan-1-yl)-1H-benzo[d]imidazole-4-carboxamide is prepared according to the procedure for Example 29, substituting cyclopropanecarbaldehyde for formaldehyde.

### Example 32

### 2-(7-methyl-7-azabicyclo[2.2.1]heptan-1-yl)-1H-benzo[d]imidazole-4-carboxamide

A solution of 2-(7-azabicyclo[2.2.1]heptan-1-yl)-1H-benzo[d]imidazole-4-carboxamide (358 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-(7-methyl-7-azabicyclo[2.2.1]heptan-1-yl)-1H-benzo[d]imidazole-4-carboxamide.

### Example 33

### 2-(7-ethyl-7-azabicyclo[2.2.1]heptan-1-yl)-1H-benzo[d]imidazole-4-carboxamide

The title compound, 2-(7-ethyl-7-azabicyclo[2.2.1]hepan-1-yl)-1H-benzo[d]imidazole-4-carboxamide is prepared according to the procedure for Example 32, substituting acetaldehyde for formaldehyde.

### Example 34

### 2-(7-cyclopropyl-7-azabicyclo[2.2.1]heptan-1-yl)-1H-benzo[d]imidazole-4-carboxamide

The title compound, 2-(2-cyclopropyl-7-azabicyclo[2.2.1]heptan-1-yl)-1H-benzo[d]imidazole-4-carboxamide is prepared according to the procedure for Example 32, substituting cyclopropanecarbaldehyde for formaldehyde.

### Example 35

### 2-((1S,5R)-6-methyl-6-azabicyclo[3.2.1]octan-5-yl)-1H-benzo[d]imidazole-4-carboxamide

A solution of 2-((1S,5R)-6-azabicyclo[3.2.1]octan-5-yl)-1H-benzo[d]imidazole-4-carboxamide (378 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-((1S,5R)-6-methyl-6-azabicyclo[3.2.1]octan-5-yl)-1H-benzo[d]imidazole-4-carboxamide.

### Example 36

### 2-((1S,5R)-6-ethyl-6-azabicyclo[3.2.1]octan-5-yl)-1H-benzo[d]imidazole-4-carboxamide

The title compound, 2-((1S,5R)-6-ethyl-6-azabicyclo[3.2.1]octan-5-yl)-1H-benzo[d]imidazole-4-carboxamide is prepared according to the procedure for Example 35, substituting acetaldehyde for formaldehyde.

### Example 37

### 2-((1S,5R)-6-propyl-6-azabicyclo[3.2.1]octan-5-yl)-1H-benzo[d]imidazole-4-carboxamide

The title compound, 2-((1S,5R)-6-propyl-6-azabicyclo[3.2.1]octan-5-yl)-1H-benzo[d]imidazole-4-carboxamide is prepared according to the procedure for Example 35, substituting butyraldehyde for formaldehyde.

### Example 38

### 2-(6-methyl-6-azabicyclo[3.2.1]octan-5-yl)-1H-benzo[d]imidazole-4-carboxamide

A solution of 2-(6-azabicyclo[3.2.1]octan-5-yl)-1H-benzo[d]imidazole-4-carboxamide (378 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-(6-methyl-6-azabicyclo[3.2.1]octan-5-yl)-1H-benzo[d]imidazole-4-carboxamide.

### Example 39

### 2-(6-ethyl-6-azabicyclo[3.2.1]octan-5-yl)-1H-benzo[d]imidazole-4-carboxamide

The title compound, 2-(6-ethyl-6-azabicyclo[3.2.1]octan-5-yl)-1H-benzo[d]imidazole-4-carboxamide is prepared according to the procedure for Example 38, substituting acetaldehyde for formaldehyde.

### Example 40

### 2-(6-pentyl-6-azabicyclo[3.2.1]octan-5-yl)-1H-benzo[d]imidazole-4-carboxamide

The title compound, 2-(6-pentyl-6-azabicyclo[3.2.1]octan-5-yl)-1H-benzo[d]imidazol-4-carboxamide is prepared according to the procedure for Example 38, substituting hexanal for formaldehyde.

### Example 41

### 2-(2-methyl-2-azabicyclo[2.2.2]octan-1-yl)-1H-benzo[d]imidazole-4-carboxamide

A solution of 2-(2-azabicyclo[2.2.2]octan-1-yl)-1H-benzo[d]imidazole-4-carboxamide (378 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-(2-methyl-2-azabicyclo[2.2.2]octan-1-yl)-1H-benzo[d]imidazole-4-carboxamide.

### Example 42

### 2-(2-ethyl-2-azabicyclo[2.2.2]octan-1-yl)-1H-benzo[d]imidazole-4-carboxamide

The title compound, 2-(2-methyl-2-azabicyclo[2.2.2]octan-1-yl)-1H-benzo[d]imidazole-4-carboxamide is prepared according to the procedure for Example 41, substituting acetaldehyde for formaldehyde.

### Example 43

### 2-(2-cyclopropyl-2-azabicyclo[2.2.2]octan-1-yl)-1H-benzo[d]imidazole-4-carboxamide

The title compound, 2-(2-cyclopropyl-2-azabicyclo[2.2.2]octan-1-yl)-1H-benzo[d]imidazole-4-carboxamide is prepared according to the procedure for Example 41, substituting cyclopropanecarbaldehyde for formaldehyde.

### Example 44

### 2-(4-methyl-4-azaspiro[2.4]heptan-5-yl)-1H-benzo[d]imidazole-4-carboxamide

A solution of 2-(4-azaspiro[2.4]heptan-5-yl)-1H-benzo[d]imidazole-4-carboxamide (358 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-(4-methyl-4-azaspiro[2.4]heptan-5-yl)-1H-benzo[d]imidazole-4-carboxamide.

### Example 45

### 2-(4-ethyl-4-azaspiro[2.4]heptan-5-yl)-1H-benzo[d]imidazole-4-carboxamide-4-carboxamide

The title compound, 2-(4-ethyl-4-azaspiro[2.4]heptan-5-yl)-1H-benzo[d]imidazole-4-carboxamide is prepared according to the procedure for Example 44, substituting acetaldehyde for formaldehyde.

### Example 46

### 2-(4-propyl-4-azaspiro[2.4]heptan-5-yl)-1H-benzo[d]imidazole-4-carboxamide

The title compound, 2-(2-propyl-2-azabicyclo[2.2.2]octan-1-yl)-1H-benzo[d]imidazole-4-carboxamide carboxamide is prepared according to the procedure for Example 44, substituting butyraldehyde for formaldehyde.

### Example 47

### 2-(2-azabicyclo[2.2.1]heptan-1-yl)-5-chloro-1H-benzo[d]imidazole-4-carboxamide

Following the experimental procedure as described in Examples 2A to 2D, and substituting 2,3-diamino-benzamide dihydrochloride in Example 2B with 2,3-diamino-5-chlorobenzamide [described in US patent application publication (US2006/0229351 A1)], the title compound, 2-(2-azabicyclo[2.2.1]heptan-1-yl)-5-chloro-1H-benzo[d]imidazole-4-carboxamide is made.

### Example 48

### 2-(7-azabicyclo[2.2.1]heptan-1-yl)-5-chloro-1H-benzo[d]imidazole-4-carboxamide

Following the experimental procedure as described in Examples 2A to 2D, and substituting 2,3-diamino-benzamide dihydrochloride in Example 2B with 2,3-diamino-5-chlorobenzamide [described in US patent application publication (US2006/0229351 A1)], and substituting (1R,4S)-2-azabicyclo[2.2.1]heptane-1-carboxylic acid, with 7-azabicyclo[2.2.1]heptane-1-carboxylic acid [described in Tetrahedron: Asymmetry, 17(2), 252-258; 2006 and Tetrahedron, 57(3) 545-548; 2001], the title compound 2-(7-azabicyclo[2.2.1]heptan-1-yl)-5-chloro-1H-benzo[d]imidazole-4-caroxamide is made.

### Example 49

### 2-(2-azabicyclo[2.1.1]hexan-1-yl)-5-chloro-1H-benzo[d]imidazole-4-carboxamide

Following the experimental procedure as described in Examples 2A to 2D, and substituting 2,3-diamino-benzamide dihydrochloride in Example 2B with 2,3-diamino-5-chlorobenzamide [described in US patent application publication (US2006/0229351 A1)], and substituting (1R,4S)-2-azabicyclo[2.2.1]heptane-1-carboxylic acid with 2-azabicyclo[2.1.1]hexane-1-carboxylic acid [described in Journal Organic Chemistry, 67, 6509-6513; 2002], the title compound 2-(2-azabicyclo[2.1.1]hexan-1-yl)-5-chloro-1H-benzo[d]imidazole-4-carboxamide is made.

### Example 50

### 2-(6-azabicyclo[3.2.1]octan-5-yl)-5-chloro-1H-benzo[d]imidazole-4-carboxamide

Following the experimental procedure as described in Examples 2A to 2D, and substituting 2,3-diamino-benzamide dihydrochloride in Example 2B with 2,3-diamino-5-chlorobenzamide [described in US patent application publication (US2006/0229351 A1)], and substituting (1R,4S)-2-azabicyclo[2.2.1]heptane-1-carboxylic acid with 6-azabicyclo[3.2.1]octane-1-carboxylic acid [described in Tetrahedron: Asymmetry, 17(2), 252-258; 2006], the title compound 2-(6-azabicyclo[3.2.1]octan-5-yl)-5-chloro-1H-benzo[d]imidazole-4-carboxamide is made.

### Example 51

### 2-(2-azabicyclo[2.2.2]octan-1-yl)-5-chloro-1H-benzo[d]imidazole-4-carboxamide

Following the experimental procedure as described in Example 11 and substituting 2,3-diamino-benzamide dihydrochloride in Example 2B with 2,3-diamino-5-chlorobenzamide [described in US patent application publication (US2006/0229351 A1)], and substituting 2-benzyl-2-azabicyclo[2.2.2]octane-4-carboxylic acid with 2-azabicyclo[2.2.2]octane-4-carboxylic acid which is synthesized by hydrogenolysis of 2-benzyl-2-azabicyclo[2.2.2]octane-4-carboxylic acid [described in Tetrahedron, 62(42), 10000-10004; 2006] to remove the benzyl group, the title compound 2-(2-azabicyclo[2.2.2]octan-1-yl)-5-chloro-1H-benzo[d]imidazole-4-carboxamide is made.

### Example 52

### 2-(4-azaspiro[2.4]heptan-5-yl)-5-chloro-1H-benzo[d]imidazole-4-carboxamide

Following the experimental procedure as described in Examples 2A to 2D, and substituting 2,3-diamino-benzamide dihydrochloride in Example 2B with 2,3-diamino-5-chlorobenzamide [described in US patent application publication (US2006/0229351 A1)], and substituting (1R,4S)-2-azabicyclo[2.2.1]heptane-1-carboxylic acid with 4-azaspiro[2.4]heptane-5-carboxylic acid [described in Tetrahedron Letters, 30(4), 399-402; 1989], the title compound 2-(4-azaspiro[2.4]heptan-5-yl)-5-chloro-1H-benzo[d]imidazole-4-carboxamide is made.

### Example 53

### 2-(1-azabicyclo[2.2.1]heptan-4-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 1-azabicyclo[2.2.1]heptane-4-carboxylic acid hydrochloride (described in Chemical Abstract, 1989, 110, 95016), and using two times the amount of triethyl amine as in Example 2A, the title compound 2-(1-azabicyclo[2.2.1]heptan-4-yl)-1H-benzo[d]imidazole-4-carboxamide is made.

### Example 54

### 2-(quinuclidin-4-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with quinuclidine-4-carboxylic acid hydrochloride (described in Helvetica Chimica Acta, 1974, 57, 2332), and using two times the amount of triethyl amine as in Example 2A, the title compound 2-(quinuclidin-4-yl)-1H-benzo[d]imidazole-4-carboxamide is made.

### Example 55

### 2-(1-azabicyclo[3.3.1]nonan-5-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 1-azabicyclo[3.3. 1]nonane-5-carboxylic acid (described U.S. Pat. No. 573,216, July 1992), the title compound 2-(1-azabicyclo[3.3.1]nonan-5-yl)-1H-benzo[d]imidazole-4-carboxamide is made.

### Example 56

### 2-(octahydro-1H-quinolizin-2-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with 1-azabicyclo[4.4.0]decane-4-carboxylic acid hydrochloride [described in P. A. Wyman et al, Bioorg. and Med. Chem. (1996), 4, 255-261], and using two times the amount of triethyl amine as in Example 2A, the title compound 2-(octahydro-1H-quinolizin-2-yl)-1H-benzo[d]imidazole-4-carboxamide is made.

### Example 57

### 2-(octahydro-1H-quinolizin-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with octahydro-1H-quinolizine-1-carboxylic acid [described in E. E. Van Tamelen et al, J. Am. Chem. Soc. (1969), 91(26), 7372-7377], the title compound 2-(octahydro-1H-quinolizin-1-yl)-1H-benzo[d]imidazole-4-carboxamide is made.

### Example 58

### 2-(octahydro-1H-quinolizin-4-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with octahydro-1H-quinolizine-4-carboxylic acid [described in R. Lukes, Chemicke Listy pro Vedu a Prumysl (1958), 52, 1608-12], the title compound 2-(octahydro-1H-quinolizin-4-yl)-1H-benzo[d]imidazole-4-carboxamide is made.

### Example 59

### 2-(octahydro-1H-quinolizin-3-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with octahydro-1H-quinolizine-4-carboxylic acid obtained by hydrolysis of ethyl octahydro-1H-quinolizine-4-carboxylate [described in Chemical & Pharmaceutical Bulletin, 27(6),1454-1463, 1979], the title compound 2-(octahydro-1H-quinolizin-3-yl)-1H-benzo[d]imidazole-4-carboxamide is made.

### Example 60

### 2-(octahydrocyclopenta[c]pyrrol-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid with octahydrocyclopenta[c]pyrrol-1-carboxylic acid [described in S. Bergmeier et al, Tetrahedron (1999), 55(26) 8025-8038], the title compound 2-(octahydrocyclopenta[c]pyrrol-1-yl)-1H-benzo[d]imidazole-4-carboxamide is made.

### Example 61

### 2-(2-ethyl-7-azabicyclo[2.2.1]heptan-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid, with 2-ethyl-7-azabicyclo[2.2.1]heptane-1-[described in Tetrahedron: Asymmetry, 14(11), 1479-1488; 2003], the title compound 2-(2-ethyl-7-azabicyclo[2.2.1]heptan-1-yl)-1H-benzo[d]imidazole-4-caroxamide is made.

### Example 62

### 2-(2-hydroxy-7-azabicyclo[2.2.1]heptan-1-yl)-1H-benzo[d]imidazole-4-carboxamide

Following the experimental procedure as described in Examples 2A to 2D, and substituting (1R,4S)-2-azabicyclo[2.2.1]heptane-1-carboxylic acid, with 2-hydroxy-7-azabicyclo[2.2.1]heptane-1-carboxylic acid hydrochloride [described in Tetrahedron: Asymmetry, 13(6), 625-632; 2002], and using two times the amount of triethyl amine as in Example 2A, the title compound 2-(2-hydroxy-7-azabicyclo[2.2.1]heptan-1-yl)-1H-benzo[d]imidazole-4-caroxamide is made.

The following compounds is made performing the experimental procedure as described in Examples 2A to 2D, and substituting (1R,4S)-2-azabicyclo[2.2.1]heptane-1-carboxylic acid with the appropriate carboxylic acid.

### Example 63

### 2-(2-azabicyclo[2.2.1]heptan-4-yl)-1H-benzo[d]imidazole-4-carboxamide

### Example 64

### 2-(2-oxa-5-azabicyclo[2.2.1]heptan-4-yl)-1H-benzo[d]imidazole -4-carboxamide

### Example 65

### 2-(2-azabicyclo[2.2.2]octan-4-yl)-1H-benzo[d]imidazole-4-carboxamide

### Example 66

### 2-(2-azabicyclo[3.2.0]heptan-1-yl)-1H-benzo[d]imidazole-4-carboxamide

### Example 67

### 2-(3-azabicyclo[3.2.0]heptan-1-yl)-1H-benzo[d]imidazole-4-carboxamide

### Example 68

### 2-(2-azabicyclo[3.2.0]heptan-4-yl)-1H-benzo[d]imidazole-4-carboxamide

### Example 69

### 2-(2-azabicyclo[3.2.0]heptan-3-yl)-1H-benzo[d]imidazole-4-carboxamide

### Example 70

### 2-(5-azaspiro[2.4]heptan-6-yl)-1H-benzo[d]imidazole-4-carboxamide

### Example 71

### 2-(4-azaspiro[2.4]heptan-6-yl)-1H-benzo[d]imidazole-4-carboxamide

### Example 72

### 2-(6-azaspiro[3.4]octan-7-yl)-1H-benzo[d]imidazole-4-carboxamide

### Example 73

### 2-(5-azaspiro[3.4]octan-6-yl)-1H-benzo[d]imidazole-4-carboxamide

### Example 74

### 2-(5-azaspiro[3.4]octan-7-yl)-1H-benzo[d]imidazole-4-carboxamide

### Example 75

### 2-(6-azaspiro[2.5]octan-5-yl)-1H-benzo[d]imidazole-4-carboxamide

### Example 76

### 2-(4-azaspiro[2.5]octan-5-yl)-1H-benzo[d]imidazole-4-carboxamide

### Example 77

### 2-(5-azaspiro[2.5]octan-7-yl)-1H-benzo[d]imidazole-4-carboxamide

### Example 78

### 2-(4-oxa-7-azaspiro[2.5]octan-6-yl)-1H-benzo[d]imidazole-4-carboxamide

### Example 79

### 2-(4-oxa-7-azaspiro[2.5]octan-5-yl)-1H-benzo[d]imidazole-4-carboxamide

### Example 80

### 2-(4-azaspiro[2.5]octan-7-yl)-1H-benzo[d]imidazole-4-carboxamide

### Example 81

### 2-(5-azaspiro[3.5]nonan-6-yl)-1H-benzo[d]imidazole-4-carboxamide

### Example 82

### 2-(6-azaspiro[3.5]nonan-8-yl)-1H-benzo[d]imidazole-4-carboxamide

### Example 83

### 2-(7-azaspiro[3.5]nonan-6-yl)-1H-benzo[d]imidazole-4-carboxamide

### Example 84

### 2-(5-azaspiro[3.5]nonan-8-yl)-1H-benzo[d]imidazole-4-carboxamide

### Example 85

### 2-(8-oxa-5-azaspiro[3.5]nonan-6-yl)-1H-benzo[d]imidazole-4-carboxamide

### Example 86

### 2-(5-oxa-5-azaspiro[3.5]nonan-6-yl)-1H-benzo[d]imidazole-4-carboxamide

### Example 87

### 2-(2,3,4,6,7,9a-hexahydro-1H-quinolizin-2-yl)-1H-benzo[d]imidazole-4-carboxamide

### Example 88

### 2-(decahydropyrido [1,2-a]azepin-4-yl)-1H-benzo[d]imidazole-4-carboxamide

### Example 89

### 2-(2-azabicyclo[2.1.1]hexan-1-yl)-1H-benzo[d]imidazole-4-carboxamide

### Example 89A

### Methyl 2-(benzyloxycarbonylamino)-3-hydroxypropanoate

To a solution of N-carbobenzyloxy-serine (50 g) in methanol (300 mL) was added thionyl chloride (17 mL, 1.2 mole equivalent) dropwise at −20° C. The reaction mixture was allowed to warm to ambient temperature and stirred overnight. The solvents were removed under reduced pressure. The residual oil was dissolved into ethyl acetate (500 mL) and washed with aqueous NaHCO₃, dried over Na₂SO₄, and concentrated under vacuum to yield (50 g, 95%) methyl 2-(benzyloxycarbonylamino)-3-hydroxypropanoate as yellow oil.

### Example 89B

### 2-(benzyloxycarbonylamino)-3-(tosyloxy)propaneate

To a solution of methyl 2-(benzyloxycarbonylamino)-3-hydroxypropanoate (50 g, 0.5 mol) in pyridine (200 mL) chilled within an ice/salt bath was added freshly purified tosylchloride (1.2 eq.) in one portion. The reaction mixture was warmed to ambient temperature slowly and stirred overnight. The reaction mixture was poured into an ice-water mixture (1200 g). A mount of solid precipitated and was collected by filtration under reduced pressure. The collected solid was dissolved into ethyl acetate, and the solution was washed with brine, dried over MgSO₄ and concentrated under reduced pressure to give 2-(benzyloxycarbonylamino)-3-(tosyloxy)propaneate (65 g, 80%) as yellowish solid.

### Example 89C

### methyl 2-(allyl(benzyloxycarbonyl)amino)acrylate

To a solution of methyl 2-(benzyloxycarbonylamino)-3-(tosyloxy)propanoate (50 g) in THF (400 mL) was added dropwise a solution of potassium tert-butoxide (33 g, 2.2 eq.) in THF (150 mL) at −20° C. under nitrogen. The reaction mixture was allowed to warm to 0° C. and stirred for 30 minutes at that temperature, and then allyl bromide (13 mL, 1.2 eq.) was added within 10 minutes. The reaction mixture was warmed naturally to room temperature, stirred for 4 h. Water was added to quench the reaction, then the mixture was extracted with ethyl acetate, washed with brine, dried over MgSO₄ and concentrated under vacuum to yield after a short silica gel chromatography methyl 2-(allyl(benzyloxycarbonyl)amino)acrylate (30 g, 89%). ¹H NMR (400 M, CDCl₃) δ (ppm): 3.75 (s, 3H), 5.17 (s 2H), 5.73 (d, 2H), 6.17 (d, 2H), 7.29 (5H, Ar); ¹³CNMR (400 M, CDCl₃) δ (ppm) 164.18, 153.13, 135.83, 130.99, 128.39 (Ar), 106.09, 67.07, 52.94.

### Example 89D

### methyl N-carbobenzyloxy-2-azabicyclo[2.1.1]hexane-1-dicarboxylate abc

A solution of methyl 2-(allyl(benzyloxycarbonyl)amino)acrylate (2.0 g) in benzene (100 mL) containing acetophenone (200 mg) was irradiated through quartz glass with a 22-W medium-pressure mercury lamp at room temperature for 6 days. The solvents were removed under vacuum, and methyl N-carbobenzyloxy-2-azabicyclo[2.1.1]hexane-1-dicarboxylate (900 mg) was isolated by silica gel flash column chromatography (silica gel, hexane/ethyl acetate=10/1→3/2). ¹H NMR (400 M, CDCl₃) δ (ppm): 1.66 (d, 2H), 2.04 (d, 2H), 2.70 (s, 3.45. 3H), 5.10 (s, 2H), 7.25 (5 H, Ar); ¹³C NMR (400 M, CDCl₃) δ (ppm) 168.81, 157.27, 136.13, 128.49, 128.16, 118.42, 70.10, 67.43, 52.02, 42.71, 34.60.

### Example 89E

### benzyl 1-(hydroxymethyl)-2-azabicyclo[2.1 1]hexane-2-carboxylate

To a solution of methyl N-carbobenzyloxy-2-azabicyclo[2.1.1]hexane-1-dicarboxylate (2.0 g, 7.26 mmol) in THF (20 mL) immersed within an ice-salt bath was added dropwise Dibal-H (15.2 mL, 20% in hexane, density: 0.848 g/L) at −20° C. with stirring. After stirred for 5 hr, the reaction was quenched with 20 mL saturated Rochelle salt. The mixture was extracted with CH₂Cl₂ (3×50 mL). The combined organic phase was washed with brine, dried on MgSO₄, and condensed under reduced pressure to yield crude, which was purified by flash column chromatography on silica gel to give benzyl 1-(hydroxymethyl)-2-azabicyclo[2.1.1]hexane-2-carboxylate (1.5 g, 67.5%) as colorless oil. ¹H NMR (400 M, CDCl₃) δ (ppm): 1.61 (d, 2H), 2.0 (2H), 2.64 (s, 1H), 3.42 (s, 2H), 3.54 (s 2H), 7.28 (m, 5H, Ar).

### Example 89F

### benzyl 1-formyl-2-azabicyclo[2.1.1]hexane-2-carboxylate

To a solution of benzyl l-(hydroxymethyl)-2-azabicyclo[2.1.1]hexane-2-carboxylate (1.78 g) in CH₂Cl₂ (20 mL) was added DMAP (3.5 g) in one portion. The mixture was stirred overnight. The solid was filtrated away. The filtrate was concentrated under reduced pressure. The residue was purified by flash column chromatography on silica gel to give benzyl 1-formyl-2-azabicyclo[2.1.1]hexane-2-carboxylate (1.20, 67.4%) as colorless oil. ¹H NMR (400 M, CDCl₃) δ (ppm): 1.56 (d, 2H), 2.04 (d, 2H), 2.76 (s, 1H), 3.48 (s, 2H), 5.12 (s, 2H), 7.29 (m, 5H, Ar).

### Example 89G

### benzyl 1-(4-carbamoyl-1H-benzo[d]imidazol-2-yl)-2-azabicyclo[2.1.1]hexane-2-carboxylate

The aldehyde benzyl 1-formyl-2-azabicyclo[2.1.1]hexane-2-carboxylate (760 mg, 2.99 mmol) and the 2,3-diaminobenzamide (669 mg, 2.99 mmol) were sequentially added with stirring to a mixture of acetic acid (3 mL) and water (3 mL) at ambient temperature. After the solid was dissolved, iodine (75 mg, 0.3 mmol) was added in one portion. The reaction mixture was stirred at ambient temperature overnight. The solvents were removed under reduced pressure. The residue was washed with aqueous NaHSO₃. The mixture was extracted with ethyl acetate, washing with brine, dried on MgSO₄ and concentrated under vacuum. The yielded residue (930 mg) was isolated by flash column chromatography (silica gel: 300-400 mesh; eluent: EtOAc/hexanes=2/1-3/1) to give benzyl 1-(4-carbamoyl-1H-benzo[d]imidazol-2-yl)-2-azabicyclo[2.1.1]hexane-2-carboxylate as an off-white solid (100 mg). Additionally, the more impure fractions was recovered and purified repeatedly. ¹H NMR (400 M, CDCl₃) δ (ppm) (major and minor isomers): 1.95 (2H), 2.35 (2H), 2.83 (1H), 3.60 (2H), 4.92 (2H), 6.16 (1H), 7.25 (m, 8H, Ar), 8.0 (br, 1H), 9.67 (br. 1H), 10.89 (br, 1H). MS (m/z): 377.1(M+H⁺).

### Example 89H

### 2-(2-azabicyclo[2.1.1]hexan-1-yl)-1H-benzo[d]imidazole-4-carboxamide

5% Pd/C (20 mg) was added into the solution of benzyl 1-(4-carbamoyl-1H-benzo[d]imidazol-2-H-benzo[d]imidazol-2-yl)-2-azabicyclo[2.1.1]hexane-2-carboxylate (280 mg) in methanol (10 mL) at ambient temperature. The reaction air was displaced with nitrogen three times. Hydrogen (1 atm) was passed into the vessel. After stirred for 5 hr, the catalyst was filtrated away. The filtrate was condensed under reduced pressure to yield 2-(2-azabicyclo[2.1.1]hexan-1-yl)-1H-benzo[d]imidazole-4-carboxamide. ¹H NMR (400 M, CDCl₃) δ (ppm): 1.70 (m, 2H), 2.25 (s, 2H), 2.85 (s, 1H), 3.21 (2H), 7.24 (t, 1H), 7.63 (q, 1H), 7.81 (d, 1H), ¹³C NMR (400 M, CDCl₃), 170.47, 154.43, 124.10, 123.39, 122.06, 117.89, 67.26, 44.46, 39.03. MS (m/z): 243.2 (M+H⁺).

### Example 90

### 2-((1R,5S)-6-azabicyclo[3.2.1]octan-5-yl)-1H-benzo[d]imidazole-4-carboxamide

### Example 90A

### bicyclo[4.1.0]heptan-2-one

To a suspension of sodium (14.5 g, 60% in oil dispersion, 0.356 mol, 1.1 eq.) in dry dimethylsulfoxide (300 mL) was added Me₃SOI (78.4 g, 0.356 mol, 1.1 eq.) carefully at room temperature within 2 h. The mixture was stirred for additional 30 minutes. After that, a solution of cyclohex-2-enone (31 g, 0.322 mol) in DMSO (30 mL) was added dropwise to the reaction mixture. The reaction bath was warmed to 50° C. and stirred for 2 h. The reaction mixture was poured into ice-water (500 mL). It was then extracted with methylene chloride, washed with brine, dried on MgSO₄ and condensed under reduced pressure to afford bicyclo[4.1.0]heptan-2-one (30 g, 85%) as yellowish oil. ¹H NMR (CDCl₃,400 MHz), δ: 0.55˜2.3 (m, 10 H).

### Example 90B

### 3-(chloromethyl)cyclohexanone

To a saturated solution (150 mL) of gaseous HCl in CH₃CN was added dropwise bicyclo[4. 1 0]heptan-2-one (30 g, 0.27 mol) at ambient temperature. After completion of addition, the reaction mixture was stirred for 2 h. GC-MS monitored that the reaction was completed. The mixture was poured into ice-water. It was extracted with CH₂Cl₂, washed with brine, dried on MgSO₄ and condensed under reduced pressure to afford 3-(chloromethyl)cyclohexanone (35 g, 88%) as brown oil. ¹H NMR (CDCl₃, 400 MHz), δ: 2.40˜3.50 (m, 2H), 2.40˜2.50(m, 1H), 2.30˜2.40(m, 1H), 2.00˜2.26(s, 4H), 1.85˜1.96 (m, 2H),1.44˜1.72 (m, 2H).

### Example 90C

### 2-hydroxy-2-methylpropanenitrile

To a solution of NaHSO₃ (572 g, 5.5 mol) in water (1 L) was added dropwise acetone (290.4 g mol) within an ice-water bath with stirring. After stirred at that temperature for 2 h, a solution of KCN (358 g, 5.5 mol) in water was added slowly. The reaction mixture was stirred overnight at ambient temperature. The reaction mixture was extracted with methylene chloride, washed with brine, dried on MgSO₄ and condensed under reduced pressure to afford 2-hydroxy-2-methylpropanenitrile (300 g) as brown oil.

### Example 90D

### (S)-2-methyl-2-(1-phenylethylamino)propanenitrile

To a solution of 2-hydroxy-2-methylpropanenitrile (192.8 g, 2.66 mol) in methanol (500 ml) was added dropwise (S)-1-phenylethanamine (357 g, 2.95 mol) at room temperature. After the addition was completed, the mixture was stirred at room temperature for 10 hours. Methanol was removed under reduced pressure. The residue was dissolved into methylene chloride, which was washed with water, dried over MgSO₄ and concentrated to give (S)-2-methyl-2-(1-phenylethylamino)propanenitrile (237 g, 47%) as yellow solid. ¹H NMR (CDCl₃,400 MHz), δ: 7.28˜7.40(m, 2H), 7.20˜7.28 (m, 2H), 7.10˜7.20 (m, 1H), 3.98˜4.09 (m,1H), 1.50˜1.62 (s, 1H), 1.42˜1.50 (s, 3H), 1.31˜1.41 (d, 3H), 0.98˜1.12(s, 3H).

### Example 90E

### (1R,5S)-6-((S)-1-phenylethyl)-6-azabicyclo[3.2.1]octane-5-carbonirile

The mixture of 3-(chloromethyl)cyclohexanone (35 g, 239 mmol) and (S)-2-methyl-2-(1-phenylethylamino)propanenitrile (45 g, 239 mmol) dissolved into CH₃CN (200 ml) was heated at reflux for 12 hours, and the reaction was monitored by TLC (Hex:EtOAc=8:1). The reaction mixture was cooled to room temperature and then was poured into ice-water containing 10% sodium hydroxide. The mixture was extracted with methylene chloride, washed with water, dried on MgSO₄, and concentrated under reduced pressure to afford a mixture of (1R,5S)-6-((S)-1-phenylethyl)-6-azabicyclo[3.2.1]octane-5-carbonirile and (1S,5R)-6-((S)-1-phenylethyl)-6-azabicyclo[3.2.1]octane-5-carbonirile. They were purified by flash column chromatography on silica gel (eluent: hexane-EtOAc=1/0-100/1) yielding (1R,5S)-6-((S)-1-phenylethyl)-6-azabicyclo[3.2.1]octane-5-carbonirile (20 g, 35%) and the (1S.5R) isomer (18 g, 32%). (1R,5S)-6-((S)-1-phenylethyl)-6-azabicyclo[3.2.1]octane-5-carbonirile : ¹H NMR (CDCl₃,400 MHz), δ: 7.23˜7.40 (m, 2H), 7.15˜7.23 (m, 2H), 7.08˜7.15 (m, 1H), 4.00˜4.12 (m, 1H), 2.90˜3.00 (m, 1H), 2.22˜2.40(m, 2H), 2.10˜2.22 (m, 2H), 1.30˜1.84 (m, 9H).

### Example 90F

### (1R,5S)-6-((S)-1-phenylethyl)-6-azabicyclo[3.2.1]octane-5-carbaldehyde

To a solution of 1R,5S)-6-((S)-1-phenylethyl)-6-azabicyclo[3.2.1]octane-5-carbonitrile (5.7 g, 23.7 mmol) in hexane (35 mL) under N₂ at 0° C., was added 1 M DIBAL-H (29 mL) dropwise. The reaction mixture was stirred for 30 min at 0° C., then the ice-salt bath was removed and the reaction mixture was stirred for 2 h. To the reaction mixture was slowly added 5% aqueous HCl (3.5 mL). The forming solid was filtered off and washed with hexane. The filtrate was concentrated. The residue oil was mixed with THF (115 mL), H₂O (11.5 mL) and H₂SO₄ (2.2 mL) and stirred at room temperature overnight. The volatiles were removed and the residue was mixed with water (100 mL) and ethyl acetate (100 mL). Then excess of NaHCO₃ was slowly added. The layers were separated. The aqueous layer was extracted with ethyl acetate. The combined organic layers were washed with water twice and dried over anhydrous Na₂SO₄. The solvent was removed to yield (1R,5S)-6-((S)-1-phenylethyl)-6-azabicyclo[3.2.1]octane-5-carbaldehyde (5.5 g, 95%) as an oil. ¹H NMR (CDCl₃,400 MHz), δ: 9.47 (s, 1H), 7.27˜7.37 (m, 2H), 7.19˜7.26 (m, 2H), 7.11˜7.19 (m, 1H), 3.84˜3.96 (m, 1H), 2.94˜3.05 (m, 1H), 2.15˜2.20 (m, 1H), 1.89˜1.02 (m, 2H), 1.73˜1.83 (m, 1H), 1.63˜1.73 (m, 1H), 1.52[1.63 (m, 1H), 1.37˜1.50 (m, 2H), 1.09˜1.20 (m, 2H), 1.00˜1.12 (m, 3H),

### Example 90G

### 2-((1R,5S)-6-((S)-1-phenylethyl)-6-azabicyclo[3.2.1]octan-5-yl)-1H-benzo[d]imidazole-4-carboxylic acid

A mixture of (1R,5S)-6-((S)-1-phenylethyl)-6-azabicyclo[3.2.1]octane-5-carbaldehyde (2.0 g, 8.2 mmol), 2,3-diaminobenzamide (1.85 g, 8.2 mmol) and KHSO₃ (2.14 g, 20.6 mmol) in DMA (30 mL) was stirred at 140° C. for 17 h. The reaction mixture was poured into ice. The resulting mixture was extracted with ethyl acetate three times. The combined organic layers were washed brine twice and dried over anhydrous Na₂SO₄. The solvent was removed and the residue was purified by column (silica gel, Hex/EtOAc/HOAc=2:1:0.01) to yield 2-((1R,5S)-6-((S)-1-phenylethyl)-6-azabicyclo[3.2.1]octan-5-yl)-1H-benzo[d]imidazole-4-carboxylic acid (930 mg, 30%). ¹H NMR (DMSO, 400 MHz), δ: 10.9˜11.2 (s,1H), 7.72˜7.80 (m, 1H), 7.80˜7.90 (m, 1H), 7.40˜7.58 (m, 2H), 7.19˜7.40 (m, 4H), 3.95˜4.05 (m, 1H), 3.12˜3.23 (m, 1H), 2.22˜2.46 (m, 4H), 1.68[1.88 (m, 2H), 1.42˜1.59 (m, 2H), 1.15˜1.26 (m, 2H), 0.69˜0.80 (m, 3H).

### Example 90H

### 2-((1R,5S)-6-((S)-1-phenylethyl)-6-azabicyclo[3.2.1]octan-5-yl)-1H-benzo[d]imidazole-4-carboxamide

A mixture of 2-((1R,5S)-6-((S)-1-phenylethyl)-6-azabicyclo[3.2.1]octan-5-yl)-1H-benzo[d]imidazole-4-carboxylic acid (460 mg, 1.23 mmol) and 1,1′-carbonyldiimidazole (250 mg, 1.54 mmol) in dry THF (8 mL) under N₂ was refluxed for 1.5 h. The reaction mixture was cooled to room temperature through which NH₃ gas (˜2 L) was then slowly bubbled through the solution by a needle. The reaction mixture was stirred at room temperature overnight. The solvent was removed and the resulting solid was dissolved in ethyl acetate, washed with brine twice and dried over anhydrous Na₂SO₄. The solvent was removed and the residue was purified by column chromatography (silica gel, CH₂Cl₂/MeOH=100/1) to yield 2-((1R,5S)-6-((S)-1-phenylethyl)-6-azabicyclo[3.2.1]octan-5-yl)-1H-benzo[d]imidazole-4-carboxamide (430 mg, 94%) as a white solid. ¹H NMR (MeOD, 400 MHz), δ: 7.82˜7.92 (d, 1H), 7.69˜7.77 (d, 1H), 7.42˜7.51 (d, 2H), 7.22˜7.35 (m, 3H), 7.14˜7.22 (m, 1H), 4.09˜4.18 (m, 1H), 3.44˜3.56 (m, 2H), 2.56˜2.68 (d, 1H), 2.34˜2.50 (m, 2H), 2.27˜2.34 (d, 2H), 1.78˜1.92 (m, 2H), 1.52˜1.64 (m, 2H), 1.25˜1.31 (s, 1H), 1.12˜1.21 (m, 2H), 0.78˜0.90 (m, 3H).

### Example 90I

### 2-((1R,5S)-6-azabicyclo[3.2.1]octan-5-yl)-1H-benzo[d]imidazole-4-carboxamide

A mixture of 2-((1R,5S)-6-((S)-1-phenylethyl)-6-azabicyclo[3.2.1]octan-5-yl)-1H-benzo[d]imidazole-4-carboxamide (530 mg, 1.42 mmol) and 10% Pd/C (wet, containing 58.3% H₂O, 300 mg) in MeOH (15 mL) was hydrogenated by H₂ under a pressure of 5 MPa (49 atm) for 75 h. The reaction mixture was filtered off. The filtrate was concentrated and the residue was purified by column chromatography (silica gel, CH₂Cl₂/MeOH=15/1) to yield 2-((1R,5S)-6-azabicyclo[3.2.1]octan-5-yl)-1yl)-1H-benzo[d]imidazole-4-carboxamide (310 mg, 81%) as awhite solid. ¹HNMR (MeOD,400 MHz), δ: 7.78˜7.88 (d, 1H), 7.57˜7.66 (d,1H), 7.19˜7.27 (m, 1H), 3.35˜3.45 (s, 1H), 3.01˜3.10 (m, 1H), 2.51˜2.59 (s, 1H), 1.55˜2.15 (m, 9H), 1.15˜1.22 (s, 1H), 1.05˜1.12 (d, 1H).

### Example 91

### 2-(1-azabicyclo[3.2.1]octan-5-yl)-1H-benzo[d]imidazole-4-carboxamide

### Example 91A

### ethyl piperidine-3-carboxylate hydrochloride

To a 2000 mL of round-bottom flask, piperidine-3-carboxylic acid (129.1 g, 1 mol) and anhydrous ethanol (1700 mL) were added under nitrogen atmosphere. The mixture was cooled to −5 degree-C. and SOCl₂ (167.0 g, 1.40 mol) was added dropwise within 2 hours. The reaction mixture was heated to reflux for 5 hours and it became clear. The reaction mixture was cooled to room temperature and solvent evaporated in vacuo, giving the title compound ethyl piperidine-3-carboxylate hydrochloride as a white solid (175.0 g Yield: 92%).

### Example 91B

### ethyl 1-(2-chloroethyl)piperidine-3-carboxylate

A solution of ethyl piperidine-3-carboxylate hydrochloride (2.0 g 10 mmol) and K₂CO₃ (3.4 g, 25 mmol) in acetone (16 mL) in a 25 mL of two-neck flask, 1-bromo-2-chloroethane (2.9 g, 20 mmol) was added in one portion while stirring at room temperature and stirred for 24 hours. The reaction completion was monitored by TLC (thin layer chromatography) (Hex/EA=2:1). Then the mixture was filtrated and concentrated in vacuo. Purification by flash chromatography, eluting with hexane/ethyl acetate (5/1) afforded the title compound ethyl 1-(2-chloroethyl)piperidine-3-carboxylate (1.7g, yield:78%).¹H NMR (400 MHz, CDCl₃): 1.22 (t, 3H, J=7.1 Hz), 1.38-1.44 (m, 1H), 1.47-1.57 (m, 1H), 1.63-1.70 (m, 1H), 1.85-1.89 (m, 1H), 2.08 (t, 1H, J=8.2 Hz), 2.24 (t, 1H, J=10.3 Hz), 2.46-2.53 (m, 1H), 2.63-2.72 (m, 3H,), 2.92 (d, 1H, J=10.9 Hz), 3.51 (t, 2H, J=7.1 Hz), 4.07 (q, 2H, J=7.1 Hz), ¹³C NMR (400 MHz, CDCl₃): 14.2, 24.4, 26.7, 40.9, 41.7, 53.7, 55.4, 60.0, 60.3, 173.9 ppm.

### Example 91C

### ethyl 1-aza-bicyclo [3.2.1]octane-5-carboxylate

A solution of diisopropylamine (13.0 g, 0.1 1 mol) in anhydrous tetrahydrofuran was cooled to −78 ° C. under Argon atmosphere. n-butyllithium (32.0 g, 0. 1 mol, 20% in hexane, precooled to −78° C.) was added in 30 min, and the mixture was stirred at −78° C. for additional 30min. N,N,N′,N′-tetramethylethylenediamine (20.5 g, 0.2 mol) was added via syringe and stirred for another 20min, ethyl 1-(2-chloroethyl)piperidine-3-carboxylate (20.0 g, 0.09 mol, dissolved in anhydrous tetrahydrofuran) was added at −78° C. via double cannula. Then the mixture was allowed to warm to room temperature and stirred for 5 hours. After the disappearance of starting material ethyl 1-(2-chloroethyl)piperidine-3-carboxylate, monitored by TLC (Hex/EA=2: 1), the solvent was removed under reduced pressure. To the residue, 50 ml of water was added and the mixture was extracted with dichloromethane. The combined dichloromethane extracts was dried over anhydrous Na₂SO₄, and the solvent was evaporated in vacuo and purified by flash chromatography eluting with 50% EA/Hex followed by 100% methanol to provide ethyl 1-aza-bicyclo[3.2.1]octane-5-carboxylate as a yellow oil (10.0 g yield: 60%.). ¹H NMR (400 MHz, CDCl₃): 1.17 (t, 3H, J=7.1 Hz), 1.40 (d, 1H, J=12.9 Hz), 1.58-1.70 (m, 1H), 1.77-1.86 (m, 3H), 2.00-2.08 (m, 1H), 2.63-2.89 (m, 5H), 2.98-3.06 (m, 1H), 4.06 (q, 2H, J=7.1 Hz), ¹³C NMR (400 MHz, CDCl₃): 14.2, 19.3, 32.8, 34.7, 49.0, 52.0, 54.4, 60.4, 63.3, 175.9 ppm.

### Example 91D

### 1-aza-bicyclo [3.2.1]octan-5-ylmethanol

Ethyl 1-aza-bicyclo[3.2.1]octane-5-carboxylate (2.0 g, 10 mmol) was placed in a 50 mL of flask, flushed with Argon for about 20 min to remove air from the flask. Freshly distilled anhydrous hexane (8 mL) was then added under Argon and cooled to −10° C. Diisobutylaluminum hydride (12 mL, 1.2 M in hexane) was added via a syringe. After 2 hours stirring, methanol was added slowly and white solid precipitated immediately. The reaction mixture was filtrated and the cake was washed with methanol (20 ml×2). The combined filtration was rotary evaporated in vacuo to give 1-aza-bicyclo[3.2.1]octan-5-ylmethanol as a yellow syrup (which turned into solid later) (1.3 g, yield: 84%). The structure was confirmed by GC-MSD, giving a MS (El) m/z 141.

### Example 91E

### 1-aza-bicyclo[3.2.1]octane-5-carbaldehyde abc

To a solution of 1-aza-bicyclo[3.2.1]octan-5-ylmethanol (2.0 g, 14 mmol) in dichloromethane (15 mL) in a 50 ml of flask, DMP (Dess-Martin periodinane, 6 g, 14 mmol) was added at room temperature. The reaction mixture became cloudy and was stirred for 4 hours. The precipitate was filtrated off and washed with dichloromethane. The filtrate was collected and solvent evaporated in vacuo under reduced pressure. The residue is purified by cation ion-exchange to give the desired compound 1-aza-bicyclo[3.2.1]octane-5-carbaldehyde which was used in the next step reaction immediately.

### Example 91F

### 2-(1-aza-bicyclo[3.2.1]octan-5-yl)-1H-benzo[d]imidazole-4-carboxylic acid

To a solution of 1-aza-bicyclo[3.2.1]octane-5-carbaldehyde (1.0 g, 7.0 mmol), 2,3-diaminobenzamide dihydrochloride (1.8 g 7.7 mmol) in a mixture of acetic acid (15 ml) and water (4 ml) in a 50 ml flask, molecular iodine (0.9 g, dissolved in acetic acid) was added dropwise at room temperature. The reaction mixture was stirred for about 8 hours. The progress of reaction was monitored by TLC (n-BuOH:EtOH:AcOH=6:1:1). After the starting material 1-aza-bicyclo[3.2.1]octane-5-carbaldehyde disappeared and a new spot (R_{f}=0.3) with strong UV appeared. The acetic acid was rotary evaporated and water was added to the residue to make a solution of around 10 ml. Na₂S₂O₃ (1.0 g) was added and stirred for 10 min, The solid was filtered and the cake was washed with MeOH (10 ml×2). The filtrate was collected and solvent evaporated. The resulting residue was purified by flash chromatography, eluting with ethyl acetate (200 ml), MeOH (200 ml) (to remove most impurity) and finally with de-ionized water (200 ml) to collect all the title substance 2-(1-aza-bicyclo[3.2.1]octan-5-yl)-1H-benzo[d]imidazole-4-carboxylic acid and the structured was confirmed by ESI-MS, +MS giving a m/z 272.1 and −MS giving a m/z 270.0

### Example 91G

### 2-(1-azabicyclo[3.2.1]octan-5-yl)-1H-benzo[d]imidazole-4-carboxamide

2-(1-aza-bicyclo[3.2.1]octan-5-yl)-1H-benzo[d]imidazole-4-carboxylic acid (50 mg, 0.15 mmol) was dissolved in dichloromethane (10 mL). O-(7-azabenzotriazol-1-yl)-N,N,N′,N′-tetramethyluronium hexafluorophoaphate (HATU) (100 mg) and diidopropylethylamine (DIPEA) (200 mg) were added with stirring. After 30 minutes, NH₃ was bubbled in the reaction mixture for 30 minutes and the reaction was stirred for additional 2 hours. The completion of the reaction was indicated by ESI-MS (+MS giving a m/z 271.2 and −MS giving a m/z 269.2). 4.0 mg of2-(1-azabicyclo[3.2.1]octan-5-yl)-1H-benzo[d]imidazole-4-carboxamide trifluoroacetic acid salt was obtained by prep-HPLC (Eluted with 15% CH₃CN-0.1% TFA H₂O).

The following compounds are made performing the experimental procedure as described in Example 29 and substituting formaldehyde with the appropriate aldehydes such as propionaldehyde, butyraldehyde, 3-(4-(cyclopropanecarbonyl)piperazin-1-yl)-3-oxopropanal, 3-(4-methylpiperazin-1-yl)-3-oxopropanal, tert-butyl 4-(3-oxopropanoyl)piperazine-1-carboxylate, 3-morpholino-3-oxopropanal, 3-morpholinopropanal, 2-morpholinoacetaldehyde, 2-(4-methylpiperazin-1-yl)acetaldehyde, 3-(4-methylpiperazine-1-yl)propanal, tert-butyl 4-(2-oxoethyl)piperazine-1-carboxylate, 5-hydroxypentanal and 5-methoxypentanal.

### Example 92

### 2-(2-propyl-2-azabicyclo[2.1.1]hexan-1-yl)-1H-benzo[d]imidazole-4-carboxamide

### Example 93

### 2-(2-butyl-2-azabicyclo[2.1.1]hexan-1-yl)-1H-benzo[d]imidazole-4-carboxamide

### Example 94

### 2-(2-(3-(4-(cyclopropanecarbonyl)piperazin-1-yl)-3-oxopropyl)-(2-azabicyclo[2.1.1]hexan-1yl)-1H-benzo[d]imidazole-4-carboxamide

### Example 95

### 2(2-(3-(4-methylpiperazin-1-yl)-3-oxopropyl)-(2-azabicyclo[2.1.1]hexan-1-yl)-1H-benzo[d]imidazole-4-carboxamide

### Example 96

### tert-butyl 4-(3-(1-(4-carbamoyl-1H-benzo[d]imidazol-2-yl)-2-azabicyclo[2.1.1]hexan-2-yl)propanoyl)piperazine-1-carboxylate

### Example 98

### 2-(2-(3-morpholino-3-oxopropyl)-2-azabicyclo[2.1.1]hexan-1-yl)-1H-benzo[d]imidazole-4-carboxamide

### Example 99

### 2-(2-(3-morpholinopropyl)-2-azabicyclo[2.1.1]hexan-1-yl)-1H-benzo[d]imidazole-4-carboxamide

### Example 100

### 2-(2-(2-morpholinoethyl)-2-azabicyclo[2.1.1]hexan-1-yl)-1H-benzo[d]imidazole-4-carboxamide

### Example 101

### 2-(2-(2-(4-methylpiperazin-1-yl)ethyl)-2-azabicyclo[2.1.1]hexan-1-yl)-1H-benzo[d]imidazole-4-carboxamide

### Example 102

### 2-(2-(3-(4-methylpiperazin-1-yl)propyl)-2-azabicyclo[2.1.1]hexan-1-yl)-1H-benzo[d]imidazole-4-carboxamide

### Example 103

### tert-butyl 4-(2-(1-(4-carbamoyl-1H-benzo[d]imidazol-2-yl)-2-azabicyclo[2.1.1]hexan-2yl)ethyl)piperazine-1-carboxylate

### Example 104

### 2-(2-(5-hydroxypentyl)-2-azabicyclo[2.1.1]hexan-1-yl)-1H-benzo[d]imidazole-4-carboxamide

### Example 105

### 2-(2-(5-methoxypentyl)-2-azabicyclo[2.1.1]hexan-1-yl)-1H-benzo[d]imidazole-4-carboxamide

### Example 106

### 2-((1R,5S)-6-methyl-6-azabicyclo[3.2.1]octan-5-yl)-1H-benzo[d]imidazole-4-carboxamide

A solution of 2-((1R,5S)-6-azabicyclo[3.2.1]octan-5-yl)-1H-benzo[d]imidazole-4-carboxamide (378 mg, 1.40 mmol) in methanol (20 mL) is treated with formaldehyde (37 wt % in water, 270 μL, 3.61 mmol) at room temperature for overnight. Sodium cyanoborohydride (228 mg, 3.61 mmol) is added and the solution is stirred at room temperature for 3 h. After concentration under reduced pressure, the residue is dissolved in a mixture of trifluoroacetic acid and water and is purified by HPLC giving the title compound 2-((1R,5S)-6-methyl-6-azabicyclo[3.2.]octan-5-yl)-1H-benzo[d]imidazole-4-carboxamide.

The following compounds are made performing the experimental procedure as described in Example 106 and substituting formaldehyde with the appropriate aldehydes such as acetaldehyde, propionaldehyde, butyraldehyde, 3-(4-(cyclopropanecarbonyl)piperazin-1-yl)-3-oxopropanal, 3-(4-methylpiperazin-1-yl)-3-oxopropanal, tert-butyl 4-(3-oxopropanoyl)piperazine-1-carboxylate, 3-morpholino-3-oxopropanal, 3-morpholinopropanal, 2-morpholinoacetaldehyde, 2-(4-methylpiperazin-1-yl)acetaldehyde, 3-(4-methylpiperazine-1yl)propanal, tert-butyl 4-(2-oxoethyl)piperazine-1-carboxylate, 5-hydroxypentanal and 5-methoxypentanal.

### Example 107

### 2-((1R,5S)-6-propyl-6-azabicyclo[3.2.1]octan-5-yl)-1H-benzo[d]imidazole-4-carboxamide

### Example 108

### 2-((1R,5S)-6-ethyl-6-azabicyclo[3.2.1]octan-5-yl)-1H-benzo[d]imidazole-4-carboxamide

### Example 109

### 2-((1R,5S)-6-butyl-6-azabicyclo[3.2.1]octan-5-yl)-1H-benzo[d]imidazole-4-carboxamide

### Example 110

### 2-((1R,5S)-6-(3-(4-(cyclopropanecarbonyl)piperazin-1-yl)-3-oxopropyl)-6-azabicyclo[3.2.1]octan-5-yl)-1H-benzo[d]imidazole-4-carboxamide

### Example 111

### 2-((1R,5S)-6-(3-(4-methylpiperazin-1-yl)-3-oxopropyl)-6-azabicyclo[3.2.1]octan-5-yl)-1H-benzo[d]imidazole-4-carboxamide

### Example 112

### tert-butyl 4-(3-((1R,5S)-5-(4-carbamoyl-1H-benzo[d]imidazol-2-yl)-6-azabicyclo[3.2.1]octan-6-yl)propanoyl)piperazine-1-carboxylate

### Example 113

### 2-((1R,5S)-6-(3-morpholino-3-oxopropyl)-6-azabicyclo[3.2.1]octan-5-yl)-1H-benzo[d]imidazole-4-carboxamide

### Example 114

### 2-((1R,5S)-6-(3-morpholinopropyl)-6-azabicyclo[3.2.1]octan-5-yl)-1H-benzo[d]imidazole-4-carboxamide

### Example 115

### 2-((1R,5S)-6-(2-morpholinoethyl)-6-azabicyclo[3.2.1]octan-5-yl)-1H-benzo[d]imidazole-4-carboxamide

### Example 116

### 2-((1R,5S)-6-(2-(4-methylpiperazin-1-yl)ethyl)-6-azabicyclo[3.2.1]octan-5-yl)-1H-benzo[d]imidazole-4-carboxamide

### Example 117

### 2-((1R,5S)-6-(3-(4-methylpiperazin-1-yl)propyl)-6-azabicyclo[3.2.1]octan-5-yl)-1H-1H-benzo[d]imidazole-4-carboxamide

### Example 118

### tert-butyl 4-(2-((1R,5S)-5-(4-carbamoyl-1H-benzo[d]imidazol-2-yl)-6-azabicyclo[3.2.1]octan-6-yl)ethyl)piperazine-1-carboxylate

### Example 119

### 2-((1R,5S)-6-(5-hydroxypentyl)-6-azabicyclo[3.2.1]octan-5-yl)-1H-benzo[d]imidazole-4-carboxamide

### Example 120

### 2-((1R,5S)-6-(5-methoxypentyl)-6-azabicyclo[3.2.1]octan-5-yl)-)-1H-benzo[d]imidazole-4-carboxamide

### Example 121

### 2-(2-(2-(piperazin-1-yl)ethyl)-2-azabicyclo[2.1.1]hexan-1-yl)-1H-benzo[d]imidazole-4-carboxamide

A solution of tert-butyl 4-(2-(1-(4-carbamoyl-1H-benzo[d]imidazol-2-yl)-2-azabicyclo[2.1.1]hexan-2-yl)ethyl)piperazine-1-carboxylate (50 mg) in trifluoroacetic acid is stirred for 30 minutes. The solvent is removed under reduced pressure giving 2-(2-(2-(piperazin-1-yl)ethyl)-2-azabicyclo[2.1.1]hexan-1-yl)-1H-benzo[d]imidazole-4-carboxamide in good yield.

### Example 122

### 2-((1R,5S)-6-(3-oxo-3-(piperazin-1-yl)propyl)-6-azabicyclo[3.2.1]octan-5-yl)-1H-benzo[d]imidazole-4-carboxamide

To a solution of tert-butyl 4-(3-((1R,5S)-5-(4-carbamoyl-1H-benzo[d]imidazol-2-yl)-6-azabicyclo[3.2.1]octan-6-yl)-)propanoyl)piperazine-1-carboxylate (50 mg) in trifluoroacetic acid is stirred for 30 minutes. The solvent is removed under reduced pressure giving 2-((1R,5S)-6-(3-oxo-3-(piperazin-1-yl)propyl)-2-azabicyclo[2.1.1]hexan-1-yl)-1H-benzo[d]imidazole-4-carboxamide in good yield.

### Example 123

### 2-((1R,5S)-6-(2-(piperazin-1-yl)ethyl)-6-azabicyclo[3.2.1]octan-5-yl)-1H-benzo[d]imidazolo-4-carboxamide

To a solution of tert-butyl 4-(2-((1R,5S)-5-(4-carbamoyl-1H-benzo[d]imidazol-6-azabicyclo[3.2.1]octan-6-yl)ethyl)piperazine-1-carboxylate (50 mg) in trifluoroacetic acid is stirred for 30 minutes. The solvent is removed under reduced pressure giving 2-((1R,5S)-6-(2-(piperazin-1-yl)ethyl)-6-azabicyclo[3.2. 1]octan-5-yl)-1H-benzo[d]imidazole-4-carboxamide in good yield.

What is claimed is:

1. A compound of Formula (I): 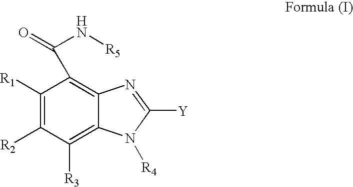
wherein:
Y is a non-aromatic 5, 6, 7, 8, 9, 10, 11, or 12-bicyclic heterocycle ring that contains 1 or 2 nitrogen atoms and, optionally, one sulfur or oxygen atom, wherein the bicyclic heterocycle is optionally substituted with 1, 2, or 3 R₆;
R₆ is selected independently from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, hydroxyl, hydroxyalkyl, nitro, oxo, heteroaryl, heteroarylalkoxy, heteroaryloxy, heteroarylthio, heteroarylalkylthio, heterocycloalkyl, heterocycloalkoxy, heterocycloalkylthio, heterocyclooxy, heterocyclothio, NR_{A}R_{B}, (NR_{A}R_{B})alkyl, (NR_{A}R_{B})carbonyl, (NR_{A}R_{B}) carbonylalkyl, and (NR_{A}R_{B})sulfonyl and is optionally attached to either one or both of the cyclic rings;
R₁, R₂, and R₃, are each independently selected from the group consisting of hydrogen, halogen, alkenyl, alkoxy, alkoxycarbonyl, alkyl, alkynyl, cyano, haloalkoxy, haloalkyl, hydroxyl, hydroxyalkyl, nitro, NR_{C}R_{D}, and (NR_{C}R_{D})carbonyl;
R_{A}, R_{B}, R_{C}, and R_{D} are independently selected from the group consisting of hydrogen, alkyl, cycloalkyl, and alkylcarbonyl; or R_{A} and R_{B} or R_{C} and R_{D} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more substituents;
R₄, and R₅ are each independently selected from the group consisting of hydrogen, alkyl, cycloalkyl, alkoxyalkyl, heterocycloalkyl, hydroxyalkyl, and (NR_{A}R_{B})alkyl; and isomers, salts, solvates, chemically protected forms, and prodrugs thereof.

2. The compound according to claim 1, wherein:
R₄, and R₅ are each independently selected from the group consisting of hydrogen, alkyl, alkoxyalkyl, heterocycloalkyl, hydroxyalkyl, and (NR_{A}R_{B})alkyl;
Y is selected from the group consisting of: 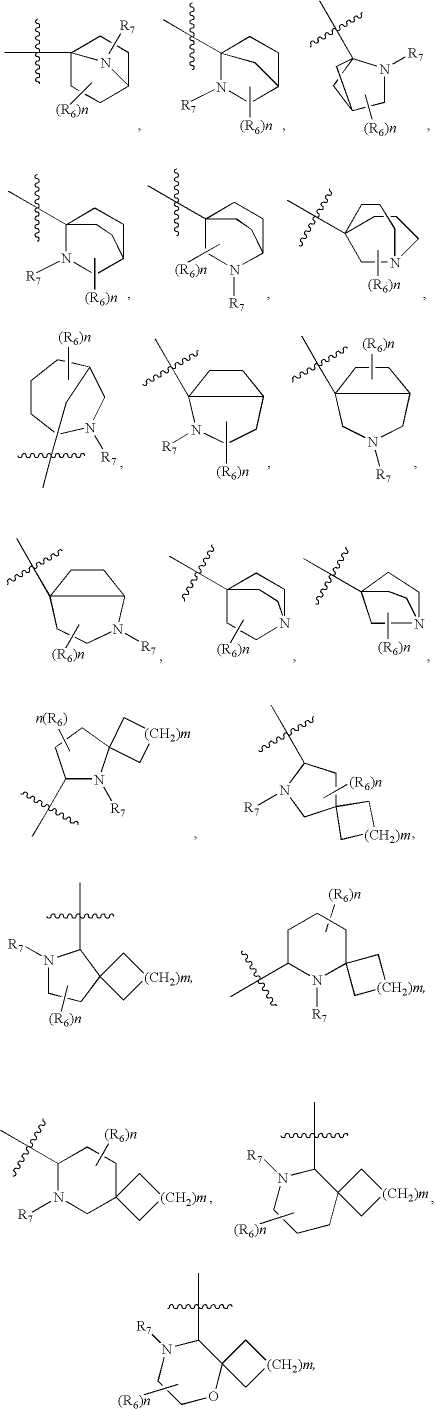 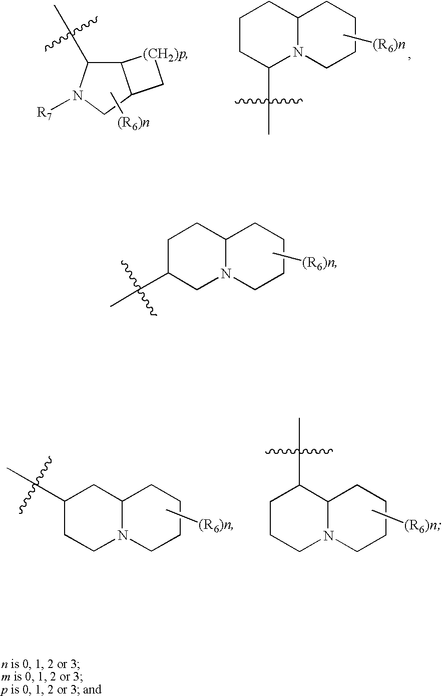
R₇ is selected from the group consisting of hydrogen, alkenyl, alkoxy, alkoxyalkyl, alkoxycarbonyl, alkoxycarbonylalkyl, alkyl, alkynyl, aryl, arylalkyl, cycloalkyl, cycloalkylalkyl, haloalkyl, hydroxyalkyl, oxo, heteroaryl, heterocycloalkylalkyl, heterocycloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonyl, (NR_{A}R_{B})carbonylalkyl, (NR_{A}R_{B})sulfonyl, and (NR_{A}R_{B})sulfonylalkyl.

3. The compound according to claim 1, wherein R₁, R₂, R₃, R₄, and R₅ are hydrogen; and n is 0.

4. The compound according to claim 2, wherein Y is selected from the group consisting of 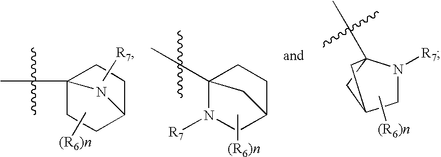
R₁, R₂, R₃, R₄, and R₅ are hydrogen;
n is 0;
R₇ is selected from the group consisting of alkoxyalkyl, alkyl, aryl, arylalkyl, cycloalkyl, haloalkyl, (NR_{A}R_{B})alkyl, alkylcarbonyl, arylcarbonyl, heteroarylcarbonyl alkylsulfonyl, arylsulfonyl, heteroarylsulfonyl, (NR_{A}R_{B})carbonylalkyl, (NR_{A}R_{B})sulfonyl, and (NR_{A}R_{B})sulfonylalkyl; and
R_{A}, and R_{B} are independently selected from the group consisting of hydrogen, alkyl, cycloalkyl, or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more R₆.

5. The compound according to claim 2, wherein Y is selected from the group consisting of 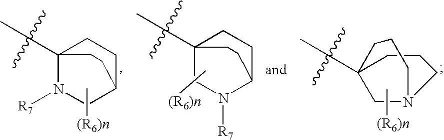
R₁, R₂, R₃, R₄, and R₅ are hydrogen;
n is 0
R₇ is selected from the group consisting of alkoxyalkyl, alkyl, aryl, arylalkyl, cycloalkyl, haloalkyl, (NR_{A}R_{B})alkyl, alkylcarbonyl, arylcarbonyl, heteroarylcarbonyl alkylsulfonyl, arylsulfonyl, heteroarylsulfonyl, (NR_{A}R_{B})carbonylalkyl, (NR_{A}R_{B})sulfonyl, and (NR_{A}R_{B})sulfonylalkyl; and
R_{A}, and R_{B} are independently selected from the group consisting of hydrogen, alkyl, cycloalkyl, or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more R₆.

6. The compound according to claim 2, wherein Y is selected from the group consisting of 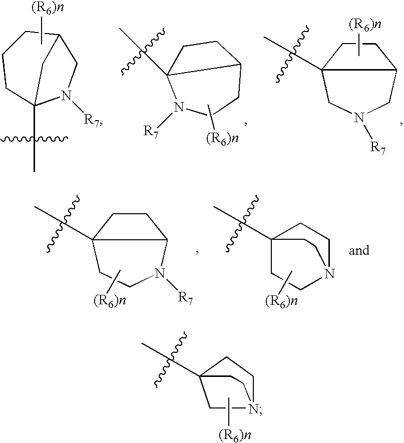
R₁, R₂, R₃, R₄, and R₅ are hydrogen;
n is 0
R₇ is selected from the group consisting of alkoxyalkyl, alkyl, aryl, arylalkyl, cycloalkyl, haloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonylalkyl, (NR_{A}R_{B})sulfonyl, and (NR_{A}R_{B})sulfonylalkyl; and
R_{A}, and R_{B} are independently selected from the group consisting of hydrogen, alkyl, cycloalkyl, or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more R₆.

7. The compound according to claim 2, wherein Y is selected from the group consisting of 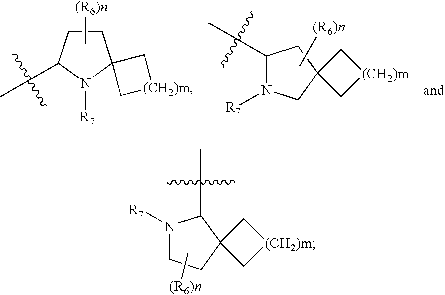
R₁, R₂, R₃, R₄, and R₅ are hydrogen;
n is 0;
R₇ is selected from the group consisting of alkoxyalkyl, alkyl, aryl, arylalkyl, cycloalkyl, haloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonylalkyl, (NR_{A}R_{B})sulfonyl, and (NR_{A}R_{B})sulfonylalkyl; and
R_{A}, and R_{B} are independently selected from the group consisting of hydrogen, alkyl, cycloalkyl,or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more R₆.

8. The compound according to claim 2, wherein Y is selected from the group consisting of 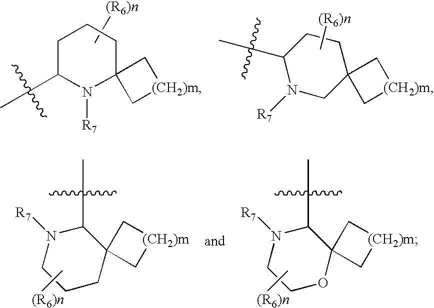
R₁, R₂, R₃, R₄, and R₅ are hydrogen;
n is 0;
R₇ is selected from the group consisting of alkoxyalkyl, alkyl, aryl, arylalkyl, cycloalkyl, haloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonylalkyl, (NR_{A}R_{B})sulfonyl, and (NR_{A}R_{B})sulfonylalkyl; and
R_{A}, and R_{B} are independently selected from the group consisting of hydrogen, alkyl, cycloalkyl, or R_{A} and R_{B} taken together with the atom to which they are attached form a 3-10 membered heterocycle ring which optionally contains one to three heteroatoms or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, -N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more R₆.

9. The compound according to claim 2, wherein Y is selected from the group consisting of 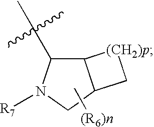
R₁, R₂, R₃, R₄, and R₅ are hydrogen;
n is 0;
R₇ is selected from the group consisting of alkoxyalkyl, alkyl, aryl, arylalkyl, cycloalkyl, haloalkyl, alkylcarbonyl, arylcarbonyl, heteroarylcarbonyl alkylsulfonyl, arylsulfonyl, heteroarylsulfonyl, (NR_{A}R_{B})alkyl, (NR_{A}R_{B})carbonylalkyl, (NR_{A}R_{B})sulfonyl, and (NR_{A}R_{B})sulfonylalkyl; and
R_{A}, and R_{B} are independently selected from the group consisting of hydrogen, alkyl, cycloalkyl, or R_{A} and R_{B} taken together with the atom to which they are attached form a 3-10 membered heterocycle ring which optionally contains one to three heteroatom or hetero functionalities selected from the group consisting of —O—, —NH, —N(C₁-C₆-alkyl)-, —NCO(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)_{q}— wherein q is 1 or 2 and the 3-10 membered heterocycle ring is optionally substituted with one or more R₆.

10. The compound according to claim 2, wherein Y is selected from the group consisting of 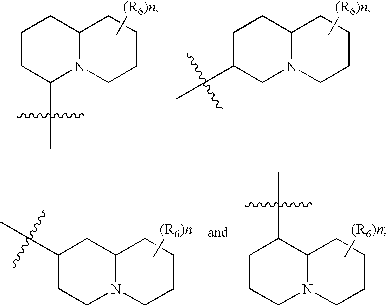
R₁, R₂, R₃, R₄, and R₅ are hydrogen; and n is 0.

11. A compound selected from the group consisting of:
2-((1R,4S)-2-azabicyclo[2.2.1]heptan-1-yl)-1H-benzo[d]imidazole-4-carboxamide;
2-((1S,4R)-2-azabicyclo[2.2.1]heptan-1-yl)-1H-benzo[d]imidazole-4-carboxamide;
2-(2-azabicyclo[2.2.1]heptan-1-yl)-1H-benzo[d]imidazole-4-carboxamide;
2-(2-azabicyclo[2.2.1]heptan-1-yl)-N-methyl-1H-benzo[d]imidazole-4-carboxamide;
2-(2-azabicyclo[2.2.1]heptan-1-yl)-1-methyl-1H-benzo[d]imidazole-4-carboxamide;
2-(7-azabicyclo[2.2.1]heptan-1-yl)-1H-benzo[d]imidazole-4-carboxamide;
2-(2-methyl-7-azabicyclo[2.2.1]heptan-1-yl)-1H-benzo[d]imidazole-4-carboxamide;
2-(2-azabicyclo[2.1.1]hexan-1-yl)-1H-benzo[d]imidazole-4-carboxamide;
2-(6-azabicyclo[3.2.1]octan-5-yl)-1H-benzo[d]imidazole-4-carboxamide;
2-((1S,5R)-6-azabicyclo[3.2.1]octan-5-yl)-1H-benzo[d]imidazole-4-carboxamide;
2-((1R,5S)-6-azabicyclo[3.2.1]octan-5-yl)-1H-benzo[d]imidazole-4-carboxamide;
2-(2-benzyl-2-azabicyclo[2.2.2]octan-1-yl)-1H-benzo[d]imidazole-4-carboxamide;
2-(2-azabicyclo[2.2.2]octan-1-yl)-1H-benzo[d]imidazole-4-carboxamide;
2-(4-azaspiro[2.4]heptan-5-yl)-1H-benzo[d]imidazole-4-carboxamide;
2-((1R,4S)-2-methyl-2-azabicyclo[2.2.1]heptan-1-yl)-1H-benzo[d]imidazole-4-carboxamide;
2-((1R,4S)-2-ethyl-2-azabicyclo[2.2.1]heptan-1-yl)-1H-benzo[d]imidazole-4-carboxamide;
2-((1R,4S)-2-cyclopropyl-2-azabicyclo[2.2.1]heptan-1-yl)-1H-benzo[d]imidazole-4-carboxamide;
2-((1R,4S)-2-isoproppyl-2-azabicyclo[2.2.1]heptan-1-yl)-1H-benzo[d]imidazole-4-carboxamide;
2-((1R,4S)-2-cyclohexyl-2-azabicyclo[2.2.1]heptan-1-yl)-1H-benzo[d]imidazole-4-carboxamide;
2-((1S,4R)-2-methyl-2-azabicyclo[2.2.1]heptan-1-yl)-1H-benzo[d]imidazole-4-carboxamide;
2-((1S,4R)-2-ethyl-2-azabicyclo[2.2.1]heptan-1-yl)-1H-benzo[d]imidazole-4-carboxamide;
2-((1S,4R)-2-cyclopropyl-2-azabicyclo[2.2.1]heptan-1-yl)-1H-benzo[d]imidazole-4-carboxamide;
2-((1R,4S)-2-proppyl-2-azabicyclo[2.2.1]heptan-1-yl)-1H-benzo[d]imidazole-4-carboxamide;
2-((1R,4S)-2-cyclobutyl-2-azabicyclo[2.2.1]heptan-1-yl)-1H-benzo[d]imidazole-4-carboxamide;
2-(2-methyl-2-azabicyclo[2.2.1]heptan-1-yl)-1H-benzo[d]imidazole-4-carboxamide;
2-(2-ethyl-2-azabicyclo[2.2.1]heptan-1-yl)-1H-benzo[d]imidazole-4-carboxamide;
2-(2-cyclopropyl-2-azabicyclo[2.2.1]heptan-1-yl)-1H-benzo[d]imidazole-4-carboxamide;
2-(2-isoproppyl-2-azabicyclo[2.2.1]heptan-1-yl)-1H-benzo[d]imidazole-4-carboxamide;
2-(2-cyclopentyl-2-azabicyclo[2.2.1]heptan-1-yl)-1H-benzo[d]imidazole-4-carboxamide;
2-(2-methyl-2-azabicyclo[2.1.1]hexan-1-yl)-1H-benzo[d]imidazole-4-carboxamide;
2-(2-ethyl-2-azabicyclo[2.1.1]hexan-1-yl)-1H-benzo[d]imidazole-4-carboxamide;
2-(2-cyclopropyl-2-azabicyclo[2.1.1]hexan-1-yl)-1H-benzo[d]imidazole-4-carboxamide;
2-(7-methyl-7-azabicyclo[2.2.1]heptan-1-yl)-1H-benzo[d]imidazole-4-carboxamide;
2-(7-ethyl-7-azabicyclo[2.2.1]heptan-1-yl)-1H-benzo[d]imidazole-4-carboxamide;
2-(7-cyclopropyl-7-azabicyclo[2.2.1]heptan-1-yl)-1H-benzo[d]imidazole-4-carboxamide;
2-((1S,5R)-6-methyl-6-azabicyclo[3.2.1]octan-5-yl)-1H-benzo[d]imidazole-4-carboxamide;
2-((1S,5R)-6-ethyl-6-azabicyclo[3.2.1]octan-5-yl)-1H-benzo[d]imidazole-4-carboxamide;
2-((1S,5R)-6-propyl-6-azabicyclo[3.2.1]octan-5-yl)-1H-benzo[d]imidazole-4-carboxamide;
2-(6-methyl-6-azabicyclo[3.2.1]octan-5-yl)1H-benzo[d]imidazole-4-carboxamide;
2-(6-ethyl-6-azabicyclo[3.2.1]octan-5-yl)-1H-benzo[d]imidazole-4-carboxamide;
2-(6-pentyl-6-azabicyclo[3.2.1]octan-5-yl)-1H-benzo[d]imidazole-4-carboxamide;
2-(2-methyl-2-azabicyclo[2.2.2]octan-1-yl)-1H-benzo[d]imidazole-4-carboxamide;
2-(2-ethyl-2-azabicyclo[2.2.2]octan-1-yl)-1H-benzo[d]imidazole-4-carboxamide;
2-(2-cyclopropyl-2-azabicyclo[2.2.2]octan-1-yl)-1H-benzo[d]imidazole-4-carboxamide;
2-(4-methyl-4-azaspiro[2.4]heptan-5-yl)-1H-benzo[d]imidazole-4-carboxamide;
2-(4-ethyl-4-azaspiro[2.4]heptan-5-yl)-1H-benzo[d]imidazole-4-carboxamide;
2-(4-propyl-4-azaspiro[2.4]heptan-5-yl)-1H-benzo[d]imidazole-4-carboxamide;
2-(2-azabicyclo[2.2.1]heptan-1-yl)-5-chloro-1H-benzo[d]imidazole-4-carboxamide;
2-(7-azabicyclo[2.2.1]heptan-1-yl)-5-chloro-1H-benzo[d]imidazole-4-carboxamide;
2-(2-azabicyclo[2.1.1]hexan-1-yl)-5-chloro-1H-benzo[d]imidazole-4-carboxamide;
2-(6-azabicyclo[3.2.1]octan-5-yl)-5-chloro-1H-benzo[d]imidazole-4-carboxamide;
2-(2-azabicyclo[2.2.2]octan-1-yl)-5-chloro-1H-benzo[d]imidazole-4-carboxamide;
2-(4-azaspiro[2.4]heptan-5-yl)-5-chloro-1H-benzo[d]imidazole-4-carboxamide;
2-(1-azabicyclo[2.2.1]heptan-4-yl)-1H-benzo[d]imidazole-4-carboxamide;
2-(quinuclidin-4-yl)-1H-benzo[d]imidazole-4-carboxamide;
2-(1-azabicyclo[3.3.1]nonan-5-yl)-1H-benzo [d]imidazole-4-carboxamide;
2-(octahydro-1H-quinolizin-2-yl)-1H-benzo[d]imidazole-4-carboxamide;
2-(octahydro-1H-quinolizin-1-yl)-1H-benzo[d]imidazole-4-carboxamide;
2-(octahydro-1H-quinolizin-4-yl)-1H-benzo[d]imidazole-4-carboxamide;
2-(octahydro-1H-quinolizin-3-yl)-1H-benzo[d]imidazole-4-carboxamide;
2-(octahydrocyclopenta[c]pyrrol-1-yl)-1H-benzo [d]imidazole-4-carboxamide;
2-(2-ethyl-7-azabicyclo[2.2.1]heptan-1-yl)-1H-benzo[d]imidazole-4-carboxamide;
2-(2-hydroxy-7-azabicyclo[2.2.1]heptan-1-yl)-1H-benzo[d]imidazole-4-carboxamide;
2-(2-azabicyclo[2.2.1]heptan-4-yl)-1H-benzo[d]imidazole-4-carboxamide;
2-(2-oxa-5-azabicyclo[2.2.1]heptan-4-yl)-1H-benzo[d]imidazole-4-carboxamide;
2-(2-azabicyclo[2.2.2]octan-4-yl)-1H-benzo[d]imidazole-4-carboxamide;
2-(2-azabicyclo[3.2.0]heptan-1-yl)-1H-benzo[d]imidazole-4-carboxamide;
2-(3-azabicyclo[3.2.0 ]heptan-1-yl)-1H-benzo[d]imidazole-4-carboxamide;
2-(2-azabicyclo[3.2.0]heptan-4-yl)-1H-benzo[d]imidazole-4-carboxamide;
2-(2-azabicyclo[3.2.0]heptan-3-yl)-1H-benzo[d]imidazole-4-carboxamide;
2-(5-azaspiro[2.4]heptan-6-yl)-1H-benzo[d]imidazole-4-carboxamide;
2-(4-azaspiro[2.4]heptan-6-yl)-1H-benzo[d]imidazole-4-carboxamide;
2-(6-azaspiro[3.4]octan-7-yl)-1H-benzo[d]imidazole-4-carboxamide;
2-(5-azaspiro[3.4]octan-6-yl)-1H-benzo[d]imidazole-4-carboxamide;
2-(5-azaspiro[3.4]octan-7-yl)-1H-benzo[d]imidazole-4-carboxamide;
2-(6-azaspiro[2.5]octan-5-yl)-1H-benzo[d]imidazole-4-carboxamide;
2-(4-azaspiro[2.5]octan-5-yl)-1H-benzo[d]imidazole-4-carboxamide;
2-(5-azaspiro[2.5]octan-7-yl)-1H-benzo[d]imidazole-4-carboxamide;
2-(4-oxa-7-azaspiro[2.5]octan-6-yl)-1H-benzo[d]imidazole-4-carboxamide;
2-(4-oxa-7-azaspiro[2.5]octan-5-yl)-1H-benzo[d]imidazole-4-carboxamide;
2-(4-azaspiro[2.5]octan-7-yl)-1H-benzo[d]imidazole-4-carboxamide;
2-(5-azaspiro[3.5]nonan-6-yl)-1H-benzo[d]imidazole-4-carboxamide;
2-(6-azaspiro[3.5]nonan-8-yl)-1H-benzo[d]imidazole-4-carboxamide;
2-(7-azaspiro[3.5]nonan-6-yl)-1H-benzo[d]imidazole-4-carboxamide;
2-(5-azaspiro[3.5]nonan-8-yl)-1H-benzo[d]imidazole-4-carboxamide;
2-(8-oxa-5-azaspiro[3.5]nonan-6-yl)-1H-benzo[d]imidazole-4-carboxamide;
2-(5-oxa-5-azaspiro[3.5]nonan-6-yl)-1H-benzo[d]imidazole-4-carboxamide;
2-(2,3,4,6,7,9a-hexahydro-1H-quinolizin-2-yl)-1H-benzo[d]imidazole-4-carboxamide;
2-(decahydropyrido [1,2-a]azepin-4-yl)-1H-benzo[d]imidazole-4-carboxamide:
2-(2-azabicyclo[2.1.1]hexan-1-yl)-1H-benzo[d]imidazole-4-carboxamide;
2-((1R,5S)-6-azabicyclo[3.2.1]octan-5-yl)-1H-benzo [d]imidazole-4-carboxamide;
2-(1-azabicyclo[3.2.1]octan-5-yl)-1H-benzo[d]imidazole-4-carboxamide:
2-(2-propyl-2-azabicyclo[2.1.1]hexan-1-yl)-1H-benzo[d]imidazole-4-carboxamide;
2-(2-butyl-2-azabicyclo[2.1.1]hexan-1-yl)-1H-benzo[d]imidazole-4-carboxamide;
2-(2-(3-(4-(cyclopropanecarbonyl)piperazin-1-yl)-3-oxopropyl)-(2-azabicyclo[2.1.1]hexan-1-yl)-1H-benzo[d]imidazole-4-carboxamide;
2(2-(3-(4-methylpiperazin-1-yl)-3-oxopropyl)-(2-azabicyclo[2.1.1]hexan-1-yl)-1H-benzo[d]imidazole-4-carboxamide;
tert-butyl 4-(3-(1-(4-carbamoyl-1H-benzo[d]imidazol-2-yl)-2-azabicyclo[2.1.1]hexan-2-yl)propanoyl)piperazine-1-carboxylate;
2-(2-(3-morpholino-3-oxopropyl)-2-azabicyclo[2.1.1]hexan-1-yl)-1H-benzo [d]imidazole-4-carboxamide;
2-(2-(3-morpholinopropyl)-2-azabicyclo[2.1.1]hexan-1-yl)-1H-benzo [d]imidazole-4-carboxamide;
2-(2-(2-morpholinoethyl)-2-azabicyclo[2.1.1]hexan-1-yl)-1H-benzo[d]imidazole-4-carboxamide;
2-(2-(2-(4-methylpiperazin-1-yl)ethyl)-2-azabicyclo[2.1.1]hexan-1-yl)-1H-benzo[d]imidazole-4-carboxamide;
2-(2-(3-(4-methylpiperazin-1-yl)propyl)-2-azabicyclo[2.1.1]hexan-1-yl)-1H-benzo[d]imidazole-4-carboxamide;
tert-butyl 4-(2-(1-(4-carbamoyl-1H-benzo[d]imidazol-2-yl)-2-azabicyclo[2.1.1]hexan-2-yl)ethyl)piperazine-1-carboxylate;
2-(2-(5-hydroxypentyl)-2-azabicyclo[2.1.1]hexan-1-yl)-1H-benzo [d]imidazole-4-carboxamide;
2-(2-(5-methoxypentyl)-2-azabicyclo[2.1.1]hexan-1-yl)-1H-benzo [d]imidazole-4-carboxamide;
2-((1R,5S)-6-methyl-6-azabicyclo[3.2.1]octan-5-yl)-1H-benzo [d]imidazole-4-carboxamide;
2-((1R,5S)-6-propyl-6-azabicyclo[3.2.1]octan-5-yl)-1H-benzo[d]imidazole-4-carboxamide;
2-((1R,5S)-6-ethyl-6-azabicyclo[3.2.1]octan-5-yl)-1H-benzo [d]imidazole-4-carboxamide;
2-((1R,5S)-6-butyl-6-azabicyclo[3.2.1]octan-5-yl)-1H-benzo [d]imidazole-4-carboxamide;
2-((1R,5S)-6-(3-(4-(cyclopropanecarbonyl)piperazin-1-yl)-3-oxopropyl)-6-azabicyclo[3.2.1]octan-5-yl)-1H-benzo[d]imidazole-4-carboxamide;
2-((1R,5S)-6-(3-(4-methylpiperazin-1-yl)-3-oxopropyl)-6-azabicyclo[3.2.1]octan-5-yl)-1H-benzo[d]imidazole-4-carboxamide;
tert-butyl 4-(3-((1R,5S)-5-(4-carbamoyl-1H-benzo [d]imidazol-2-yl)-6-azabicyclo[3.2.1]octan-6- yl)propanoyl)piperazine-1-carboxylate;
2-((1R,5S)-6-(3-morpholino-3-oxopropyl)-6-azabicyclo[3.2.1]octan-5-yl)-1H-benzo[d]imidazole-4-carboxamide;
2-((1R,5S) -6-(3-morpholinopropyl)-6-azabicyclo[3.2.1]octan-5-yl)-1H-benzo[d]imidazole-4-carboxamide;
2-((1R,5S)-6-(2-morpholinoethyl)-6-azabicyclo[3.2.1]octan-5-yl)-1H-benzo[d]imidazole-4-carboxamide;
2-((1R,5S)-6-(2-(4-methylpiperazin-1-yl)ethyl)-6-azabicyclo[3.2.1]octan-5-yl)-1H-benzo[d]imidazole-4-carboxamide;
2-((1R,5S)-6-(3-(4-methylpiperazin-1-yl)propyl)-6-azabicyclo[3.2.1]octan-5-yl)-1H-benzo[d]imidazole-4-carboxamide;
tert-butyl 4-(2-((1R,5S)-5-(4-carbamoyl-1H-benzo [d]imidazol-2-yl)-6-azabicyclo[3.2.1]octan-6-yl)ethyl)piperazine-1-carboxylate;
2-((1R,5S)-6-(5-hydroxypentyl)-6-azabicyclo[3.2.1]octan-5-yl)-1H-benzo [d]imidazole-4-carboxamide;
2-((1R,5S)-6-(5-methoxypentyl)-6-azabicyclo[3.2.1]octan-5-yl)-)-1H-benzo[d]imidazole-4-carboxamide;
2-(2-(2-(piperazin-1-yl)ethyl)-2-azabicyclo[2.1.1]hexan-1-yl)-1H-benzo[d]imidazole-4-carboxamide;
2-((1R,5S)-6-(3-oxo-3-(piperazin-1-yl)propyl)-6-azabicyclo[3.2.1]octan-5-yl)-1H-benzo[d]imidazole-4-carboxamide; and
2-((1R,5S)-6-(2-(piperazin-1-yl)ethyl)-6-azabicyclo[3.2.1]octan-5-yl)-1H-benzo [d]imidazole-4-carboxamide.

12. A pharmaceutical composition comprising a compound of claim 1 or a pharmaceutically acceptable salt, pharmaceutically acceptable solvate, or pharmaceutically acceptable prodrug thereof and a pharmaceutically acceptable carrier, excipient, binder or diluent.

13. A method of treatment of disease ameliorated by the inhibition of PARP comprising administering to a subject in need of treatment a therapeutically-effective amount of a compound of claim 1, wherein the disease is selected from the group consisting of: vascular disease; septic shock; ischaemic injury; reperfusion injury; neurotoxicity; haemorraghic shock; inflammatory diseases; multiple sclerosis; secondary effects of diabetes; and acute treatment of cytoxicity following cardiovascular surgery.

14. A method of treatment of cancer, comprising administering to a subject in need of treatment a therapeutically-effective amount of a compound according to claim 1 in combination with ionizing radiation or one or more chemotherapeutic agents.

15. The method of claim 14, wherein the compound according to claim 1 is administered simultaneously or sequentially with ionizing radiation or one or more chemotherapeutic agents.

16. A method of treatment of a cancer deficient in Homologous Recombination (HR) dependent DNA double strand break (DSB) repair pathway, comprising administering to a subject in need of treatment a therapeutically-effective amount of a compound according to claim 1.

17. A method according to claim 16, wherein said cancer comprises one or more cancer cells having a reduced or abrogated ability to repair DNA DSB by HR relative to normal cells.

18. A method according to claim 17, wherein said cancer cells have a BRCA1 or BRCA2 deficient phenotype.

19. A method according to claim 16, wherein said subject is heterozygous for a mutation in a gene encoding a component of the HR dependent DNA DSB repair pathway.

20. A method according to claim 16, wherein said subject is heterozygous for a mutation in BRCA1 and/or BRCA2.

21. A method according to claim 16, wherein said cancer is breast, ovary, pancreas or prostate cancer.

22. An article of manufacture, comprising packaging material, a compound of claim 1 which is effective for modulating the activity of the enzyme poly(ADP-ribose)polymerase, or for treatment, prevention or amelioration of one or more symptoms of a poly(ADP-ribose)polymerase-dependent or poly(ADP-ribose)polymerase-mediated disease or condition, within the packaging material, and a label that indicates that the compound or composition, or pharmaceutically acceptable salt, pharmaceutically acceptable N-oxide, pharmaceutically active metabolite, pharmaceutically acceptable pro drug, or pharmaceutically acceptable solvate thereof, is used for modulating the activity of poly(ADP-ribose)polymerase, or for treatment, prevention or amelioration of one or more symptoms of a poly(ADP-ribose)polymerase-dependent or poly(ADP-ribose)polymerase-mediated disease or condition.
